SEQUENCE LISTING (163 sequences)

SEQ ID NO: 1 - protein, 9 aa, Artificial
  an artificially synthesized peptide sequence
EYMKQLVKL

SEQ ID NO: 2 - protein, 9 aa, Artificial
  an artificially synthesized peptide sequence
LYIGHLSEL

SEQ ID NO: 3 - protein, 9 aa, Artificial
  an artificially synthesized peptide sequence
LYPSHEKLL

SEQ ID NO: 4 - protein, 9 aa, Artificial
  an artificially synthesized peptide sequence
MYLHCCLTL

SEQ ID NO: 5 - protein, 9 aa, Artificial
  an artificially synthesized peptide sequence
YYYQVYSIL

SEQ ID NO: 6 - protein, 9 aa, Artificial
  an artificially synthesized peptide sequence
AYSKLLSHL

SEQ ID NO: 7 - protein, 9 aa, Artificial
  an artificially synthesized peptide sequence
VYGHQFMNL

SEQ ID NO: 8 - protein, 9 aa, Artificial
  an artificially synthesized peptide sequence
SYTIFLCIL

SEQ ID NO: 9 - protein, 9 aa, Artificial
  an artificially synthesized peptide sequence
RYYYQVYSI

SEQ ID NO: 10 - protein, 9 aa, Artificial
  an artificially synthesized peptide sequence
MYAQKNLDI

SEQ ID NO: 11 - protein, 9 aa, Artificial
  an artificially synthesized peptide sequence
LFPPSWHLL

SEQ ID NO: 12 - protein, 9 aa, Artificial
  an artificially synthesized peptide sequence
LFEEHCETL

SEQ ID NO: 13 - protein, 9 aa, Artificial
  an artificially synthesized peptide sequence
PYEALEAQL

SEQ ID NO: 14 - protein, 9 aa, Artificial
  an artificially synthesized peptide sequence
TFLTDSLEL

SEQ ID NO: 15 - protein, 9 aa, Artificial
  an artificially synthesized peptide sequence
AFVTSQRAL

SEQ ID NO: 16 - protein, 9 aa, Artificial
  an artificially synthesized peptide sequence
NFFSLFLLL

SEQ ID NO: 17 - protein, 9 aa, Artificial
  an artificially synthesized peptide sequence
LFRIIDCLL

SEQ ID NO: 18 - protein, 9 aa, Artificial
  an artificially synthesized peptide sequence
SFWNWLNKL

SEQ ID NO: 19 - protein, 9 aa, Artificial
  an artificially synthesized peptide sequence
LFHLFRQQL

SEQ ID NO: 20 - protein, 9 aa, Artificial
  an artificially synthesized peptide sequence
LFRQQLYNL

SEQ ID NO: 21 - protein, 9 aa, Artificial
  an artificially synthesized peptide sequence
RYLSHVLQN

SEQ ID NO: 22 - protein, 9 aa, Artificial
  an artificially synthesized peptide sequence
LFLHYVKVF

SEQ ID NO: 23 - protein, 9 aa, Artificial
  an artificially synthesized peptide sequence
AFREKAKEF

SEQ ID NO: 24 - protein, 9 aa, Artificial
  an artificially synthesized peptide sequence
KNLSGPDDL

SEQ ID NO: 25 - protein, 9 aa, Artificial
  an artificially synthesized peptide sequence
KQLVKLTRL

SEQ ID NO: 26 - protein, 9 aa, Artificial
  an artificially synthesized peptide sequence
RLILNLDPL

SEQ ID NO: 27 - protein, 9 aa, Artificial
  an artificially synthesized peptide sequence
IYIDGVQEV

SEQ ID NO: 28 - protein, 9 aa, Artificial
  an artificially synthesized peptide sequence
KSAMVTKSL

SEQ ID NO: 29 - protein, 9 aa, Artificial
  an artificially synthesized peptide sequence
HVLDLLNFL

SEQ ID NO: 30 - protein, 9 aa, Artificial
  an artificially synthesized peptide sequence
LVLEILYML

SEQ ID NO: 31 - protein, 9 aa, Artificial
  an artificially synthesized peptide sequence
SFSISWLPF

SEQ ID NO: 32 - protein, 10 aa, Artificial
  an artificially synthesized peptide sequence
RYYYQVYSIL

SEQ ID NO: 33 - protein, 10 aa, Artificial
  an artificially synthesized peptide sequence
IYIDGVQEVF

SEQ ID NO: 34 - protein, 10 aa, Artificial
  an artificially synthesized peptide sequence
YYSKNLNSSF

SEQ ID NO: 35 - protein, 10 aa, Artificial
  an artificially synthesized peptide sequence
EYLGEVLKYI

SEQ ID NO: 36 - protein, 10 aa, Artificial
  an artificially synthesized peptide sequence
LYKSSSSYTI

SEQ ID NO: 37 - protein, 10 aa, Artificial
  an artificially synthesized peptide sequence
KFHQKRMEEL

SEQ ID NO: 38 - protein, 10 aa, Artificial
  an artificially synthesized peptide sequence
LFEEHCETLL

SEQ ID NO: 39 - protein, 10 aa, Artificial
  an artificially synthesized peptide sequence
AFVNQNQIKL

SEQ ID NO: 40 - protein, 10 aa, Artificial
  an artificially synthesized peptide sequence
QFMNLASDNL

SEQ ID NO: 41 - protein, 10 aa, Artificial
  an artificially synthesized peptide sequence
IFATSKAQKL

SEQ ID NO: 42 - protein, 10 aa, Artificial
  an artificially synthesized peptide sequence
RYLSHVLQNS

SEQ ID NO: 43 - protein, 10 aa, Artificial
  an artificially synthesized peptide sequence
IYSKFHQKRM

SEQ ID NO: 44 - protein, 10 aa, Artificial
  an artificially synthesized peptide sequence
SFWNWLNKLL

SEQ ID NO: 45 - protein, 10 aa, Artificial
  an artificially synthesized peptide sequence
AFREKAKEFL

SEQ ID NO: 46 - protein, 10 aa, Artificial
  an artificially synthesized peptide sequence
IFSKAQVEYL

SEQ ID NO: 47 - protein, 10 aa, Artificial
  an artificially synthesized peptide sequence
LFRIIDCLLL

SEQ ID NO: 48 - protein, 10 aa, Artificial
  an artificially synthesized peptide sequence
NFEEDTLEIF

SEQ ID NO: 49 - protein, 10 aa, Artificial
  an artificially synthesized peptide sequence
KSLEYLGEVL

SEQ ID NO: 50 - protein, 10 aa, Artificial
  an artificially synthesized peptide sequence
KLLFRIIDCL

SEQ ID NO: 51 - protein, 10 aa, Artificial
  an artificially synthesized peptide sequence
RYLKVQNAES

SEQ ID NO: 52 - protein, 10 aa, Artificial
  an artificially synthesized peptide sequence
KLLNDGFSML

SEQ ID NO: 53 - protein, 10 aa, Artificial
  an artificially synthesized peptide sequence
KQLVKLTRLL

SEQ ID NO: 54 - protein, 10 aa, Artificial
  an artificially synthesized peptide sequence
RMEELTEVGL

SEQ ID NO: 55 - protein, 10 aa, Artificial
  an artificially synthesized peptide sequence
RQQLYNLETL

SEQ ID NO: 56 - protein, 10 aa, Artificial
  an artificially synthesized peptide sequence
KNLSGPDDLL

SEQ ID NO: 57 - protein, 10 aa, Artificial
  an artificially synthesized peptide sequence
KSISVQGVIL

SEQ ID NO: 58 - protein, 10 aa, Artificial
  an artificially synthesized peptide sequence
RYDKVRSSES

SEQ ID NO: 59 - protein, 10 aa, Artificial
  an artificially synthesized peptide sequence
SFLQAVLARI

SEQ ID NO: 60 - protein, 10 aa, Artificial
  an artificially synthesized peptide sequence
LFLHYVKVFI

SEQ ID NO: 61 - protein, 10 aa, Artificial
  an artificially synthesized peptide sequence
LLLPGILKCL

SEQ ID NO: 62 - protein, 9 aa, Artificial Sequence
  an artificially synthesized peptide sequence
YLGEVLKYI

SEQ ID NO: 63 - protein, 9 aa, Artificial Sequence
  an artificially synthesized peptide sequence
KLFPPSWHL

SEQ ID NO: 64 - protein, 9 aa, Artificial Sequence
  an artificially synthesized peptide sequence
MLGEKLKQV

SEQ ID NO: 65 - protein, 9 aa, Artificial Sequence
  an artificially synthesized peptide sequence
KLTRLLFNL

SEQ ID NO: 66 - protein, 9 aa, Artificial Sequence
  an artificially synthesized peptide sequence
VLFLHYVKV

SEQ ID NO: 67 - protein, 9 aa, Artificial Sequence
  an artificially synthesized peptide sequence
SLFLLLAAV

SEQ ID NO: 68 - protein, 9 aa, Artificial Sequence
  an artificially synthesized peptide sequence
RLLFNLSEV

SEQ ID NO: 69 - protein, 9 aa, Artificial Sequence
  an artificially synthesized peptide sequence
CLCIKELWV

SEQ ID NO: 70 - protein, 9 aa, Artificial Sequence
  an artificially synthesized peptide sequence
KLLNDGFSM

SEQ ID NO: 71 - protein, 9 aa, Artificial Sequence
  an artificially synthesized peptide sequence
YLGKKVFSA

SEQ ID NO: 72 - protein, 9 aa, Artificial Sequence
  an artificially synthesized peptide sequence
WLVLEILYM

SEQ ID NO: 73 - protein, 9 aa, Artificial Sequence
  an artificially synthesized peptide sequence
TLSDKSAMV

SEQ ID NO: 74 - protein, 9 aa, Artificial Sequence
  an artificially synthesized peptide sequence
LLNDGFSML

SEQ ID NO: 75 - protein, 9 aa, Artificial Sequence
  an artificially synthesized peptide sequence
FLHYVKVFI

SEQ ID NO: 76 - protein, 9 aa, Artificial Sequence
  an artificially synthesized peptide sequence
ALVRFFEAV

SEQ ID NO: 77 - protein, 9 aa, Artificial Sequence
  an artificially synthesized peptide sequence
WLNKLLKTL

SEQ ID NO: 78 - protein, 9 aa, Artificial Sequence
  an artificially synthesized peptide sequence
TVLSFLQAV

SEQ ID NO: 79 - protein, 9 aa, Artificial Sequence
  an artificially synthesized peptide sequence
TLLCDLISL

SEQ ID NO: 80 - protein, 9 aa, Artificial Sequence
  an artificially synthesized peptide sequence
LLFRIIDCL

SEQ ID NO: 81 - protein, 9 aa, Artificial Sequence
  an artificially synthesized peptide sequence
KLLFRIIDC

SEQ ID NO: 82 - protein, 9 aa, Artificial Sequence
  an artificially synthesized peptide sequence
LLMYAQKNL

SEQ ID NO: 83 - protein, 9 aa, Artificial Sequence
  an artificially synthesized peptide sequence
LLSIYIDGV

SEQ ID NO: 84 - protein, 9 aa, Artificial Sequence
  an artificially synthesized peptide sequence
FLCILAKVV

SEQ ID NO: 85 - protein, 9 aa, Artificial Sequence
  an artificially synthesized peptide sequence
SMLEMVKTC

SEQ ID NO: 86 - protein, 9 aa, Artificial Sequence
  an artificially synthesized peptide sequence
SLFEEHCET

SEQ ID NO: 87 - protein, 9 aa, Artificial Sequence
  an artificially synthesized peptide sequence
FLQAVLARI

SEQ ID NO: 88 - protein, 9 aa, Artificial Sequence
  an artificially synthesized peptide sequence
LMSDQCPCL

SEQ ID NO: 89 - protein, 9 aa, Artificial Sequence
  an artificially synthesized peptide sequence
YLNQLLGNV

SEQ ID NO: 90 - protein, 9 aa, Artificial Sequence
  an artificially synthesized peptide sequence
SILAFILQL

SEQ ID NO: 91 - protein, 9 aa, Artificial Sequence
  an artificially synthesized peptide sequence
LLPGILKCL

SEQ ID NO: 92 - protein, 9 aa, Artificial Sequence
  an artificially synthesized peptide sequence
VLQMYIKNL

SEQ ID NO: 93 - protein, 9 aa, Artificial Sequence
  an artificially synthesized peptide sequence
GILKCLVLV

SEQ ID NO: 94 - protein, 9 aa, Artificial Sequence
  an artificially synthesized peptide sequence
LQNFFSLFL

SEQ ID NO: 95 - protein, 9 aa, Artificial Sequence
  an artificially synthesized peptide sequence
LLLPGILKC

SEQ ID NO: 96 - protein, 9 aa, Artificial Sequence
  an artificially synthesized peptide sequence
QLYNLETLL

SEQ ID NO: 97 - protein, 9 aa, Artificial Sequence
  an artificially synthesized peptide sequence
VLAEKFSCA

SEQ ID NO: 98 - protein, 9 aa, Artificial Sequence
  an artificially synthesized peptide sequence
ILYMLGEKL

SEQ ID NO: 99 - protein, 9 aa, Artificial Sequence
  an artificially synthesized peptide sequence
YLHCCLTLC

SEQ ID NO: 100 - protein, 10 aa, Artificial Sequence
  an artificially synthesized peptide sequence
ALWRHFFSFL

SEQ ID NO: 101 - protein, 10 aa, Artificial Sequence
  an artificially synthesized peptide sequence
FLLLAAVAEV

SEQ ID NO: 102 - protein, 10 aa, Artificial Sequence
  an artificially synthesized peptide sequence
YMLGEKLKQV

SEQ ID NO: 103 - protein, 10 aa, Artificial Sequence
  an artificially synthesized peptide sequence
LLDHRSKWFV

SEQ ID NO: 104 - protein, 10 aa, Artificial Sequence
  an artificially synthesized peptide sequence
FVSESFWNWL

SEQ ID NO: 105 - protein, 10 aa, Artificial Sequence
  an artificially synthesized peptide sequence
ILWEYYSKNL

SEQ ID NO: 106 - protein, 10 aa, Artificial Sequence
  an artificially synthesized peptide sequence
LLNFLKPAFV

SEQ ID NO: 107 - protein, 10 aa, Artificial Sequence
  an artificially synthesized peptide sequence
LLNDGFSMLL

SEQ ID NO: 108 - protein, 10 aa, Artificial Sequence
  an artificially synthesized peptide sequence
LIWKGHMAFL

SEQ ID NO: 109 - protein, 10 aa, Artificial Sequence
  an artificially synthesized peptide sequence
KLFPPSWHLL

SEQ ID NO: 110 - protein, 10 aa, Artificial Sequence
  an artificially synthesized peptide sequence
GLQNFFSLFL

SEQ ID NO: 111 - protein, 10 aa, Artificial Sequence
  an artificially synthesized peptide sequence
FLTDSLELEL

SEQ ID NO: 112 - protein, 10 aa, Artificial Sequence
  an artificially synthesized peptide sequence
FLLMYAQKNL

SEQ ID NO: 113 - protein, 10 aa, Artificial Sequence
  an artificially synthesized peptide sequence
QQCVLFLHYV

SEQ ID NO: 114 - protein, 10 aa, Artificial Sequence
  an artificially synthesized peptide sequence
TLLSIYIDGV

SEQ ID NO: 115 - protein, 10 aa, Artificial Sequence
  an artificially synthesized peptide sequence
LLYIGHLSEL

SEQ ID NO: 116 - protein, 10 aa, Artificial Sequence
  an artificially synthesized peptide sequence
YLCSGALKRL

SEQ ID NO: 117 - protein, 10 aa, Artificial Sequence
  an artificially synthesized peptide sequence
WLVLEILYML

SEQ ID NO: 118 - protein, 10 aa, Artificial Sequence
  an artificially synthesized peptide sequence
QLTSVFRQFI

SEQ ID NO: 119 - protein, 10 aa, Artificial Sequence
  an artificially synthesized peptide sequence
MLGEKLKQVV

SEQ ID NO: 120 - protein, 10 aa, Artificial Sequence
  an artificially synthesized peptide sequence
SLFEEHCETL

SEQ ID NO: 121 - protein, 10 aa, Artificial Sequence
  an artificially synthesized peptide sequence
FLVSKNEEMV

SEQ ID NO: 122 - protein, 10 aa, Artificial Sequence
  an artificially synthesized peptide sequence
CLCIKELWVL

SEQ ID NO: 123 - protein, 10 aa, Artificial Sequence
  an artificially synthesized peptide sequence
LLCDLISLSL

SEQ ID NO: 124 - protein, 10 aa, Artificial Sequence
  an artificially synthesized peptide sequence
LLFRIIDCLL

SEQ ID NO: 125 - protein, 10 aa, Artificial Sequence
  an artificially synthesized peptide sequence
LLPGILKCLV

SEQ ID NO: 126 - protein, 10 aa, Artificial Sequence
  an artificially synthesized peptide sequence
IQWVTETALV

SEQ ID NO: 127 - protein, 10 aa, Artificial Sequence
  an artificially synthesized peptide sequence
WLNKLLKTLL

SEQ ID NO: 128 - protein, 10 aa, Artificial Sequence
  an artificially synthesized peptide sequence
LLGNVIEQYI

SEQ ID NO: 129 - protein, 10 aa, Artificial Sequence
  an artificially synthesized peptide sequence
AMVTKSLEYL

SEQ ID NO: 130 - protein, 10 aa, Artificial Sequence
  an artificially synthesized peptide sequence
QLADAAADFT

SEQ ID NO: 131 - protein, 10 aa, Artificial Sequence
  an artificially synthesized peptide sequence
TIFLCILAKV

SEQ ID NO: 132 - protein, 10 aa, Artificial Sequence
  an artificially synthesized peptide sequence
SLMSDQCPCL

SEQ ID NO: 133 - protein, 10 aa, Artificial Sequence
  an artificially synthesized peptide sequence
WLPFKGLANT

SEQ ID NO: 134 - protein, 10 aa, Artificial Sequence
  an artificially synthesized peptide sequence
SIYIDGVQEV

SEQ ID NO: 135 - protein, 10 aa, Artificial Sequence
  an artificially synthesized peptide sequence
CLVLVSEPQV

SEQ ID NO: 136 - protein, 10 aa, Artificial Sequence
  an artificially synthesized peptide sequence
CLYPSHEKLL

SEQ ID NO: 137 - protein, 10 aa, Artificial Sequence
  an artificially synthesized peptide sequence
TIWTLLSIYI

SEQ ID NO: 138 - protein, 10 aa, Artificial Sequence
  an artificially synthesized peptide sequence
TLLAMDMPST

SEQ ID NO: 139 - protein, 10 aa, Artificial Sequence
  an artificially synthesized peptide sequence
RLATENLQYM

SEQ ID NO: 140 - protein, 10 aa, Artificial Sequence
  an artificially synthesized peptide sequence
TLEIFGIQWV

SEQ ID NO: 141 - protein, 10 aa, Artificial Sequence
  an artificially synthesized peptide sequence
MVQRQTIWTL

SEQ ID NO: 142 - protein, 10 aa, Artificial Sequence
  an artificially synthesized peptide sequence
SMLEMVKTCC

SEQ ID NO: 143 - protein, 10 aa, Artificial Sequence
  an artificially synthesized peptide sequence
YLQGMCIVCC

SEQ ID NO: 144 - protein, 10 aa, Artificial Sequence
  an artificially synthesized peptide sequence
ILEEQLRMYL

SEQ ID NO: 145 - protein, 10 aa, Artificial Sequence
  an artificially synthesized peptide sequence
SLSLNRYDKV

SEQ ID NO: 146 - protein, 10 aa, Artificial Sequence
  an artificially synthesized peptide sequence
RLASILAFIL

SEQ ID NO: 147 - protein, 10 aa, Artificial Sequence
  an artificially synthesized peptide sequence
LQNFFSLFLL

SEQ ID NO: 148 - protein, 10 aa, Artificial Sequence
  an artificially synthesized peptide sequence
VLLALRNTAT

SEQ ID NO: 149 - protein, 10 aa, Artificial Sequence
  an artificially synthesized peptide sequence
KALVRFFEAV

SEQ ID NO: 150 - protein, 10 aa, Artificial Sequence
  an artificially synthesized peptide sequence
TLCDFWEPNI

SEQ ID NO: 151 - protein, 10 aa, Artificial Sequence
  an artificially synthesized peptide sequence
LMYAQKNLDI

SEQ ID NO: 152 - DNA, 21 bp, Artificial
  An artificially synthesized primer sequence for RT-PCR
gaggtgatagcattgctttcg

SEQ ID NO: 153 - DNA, 21 bp, Artificial
  An artificially synthesized primer sequence for RT-PCR
caagtcagtgtacaggtaagc

SEQ ID NO: 154 - DNA, 20 bp, Artificial
  An artificially synthesized primer sequence for RT-PCR
gtctcaccttggacagatgg

SEQ ID NO: 155 - DNA, 24 bp, Artificial
  An artificially synthesized primer sequence for RT-PCR
ccaaggatcctattacacagttgc

SEQ ID NO: 156 - DNA, 20 bp, Artificial
  An artificially synthesized primer sequence for preparing probe
ctggaagaggcagttgaaaa

SEQ ID NO: 157 - DNA, 20 bp, Artificial
  An artificially synthesized primer sequence for preparing probe
atcgcccaatatactgctca

SEQ ID NO: 158 - DNA, 8643 bp, Homo sapiens
  CDS (267)..(3998)
gtttgcggtttagaccccaaagattcctgttggtggtctgggtcacaggaggcaggtttcgggagctggaaatgtgagcgggtacgacaggcaccgcgggtaaccgacgccccgggtccttgctgcagccgggtacgcgggataccggcaccccgccttctccgcccgagtgctgccaggcgtgggcctggaatctcttcacaccttctctttggagcccttaatgatacgacgaaccccaagtgtttcagaacatgaagtaaacaatggagaactgttctgctgcatcgacgttcctgactgacagcttagagctggagctggggacggaatggtgcaaacctccttacttttcttgtgctgttgacaacagaggaggaggaaaacatttttctggagaatcctacctctgcagcggagcccttaagcgattgattttgaatcttgaccctttaccaactaattttgaagaagataccttggaaatatttggcattcagtgggttactgaaacagcattagtgaattcatctagagaactctttcatttattcaggcaacaactgtacaacttggaaaccttgttacagtccagttgtgattttgggaaggtatcaactctacactgcaaagcagacaatattaggcagcagtgtgtactatttctccattatgttaaagttttcatcttcaggtatctgaaagtacagaatgctgagagtcatgttcctgtccatccttatgaggctttggaggctcagcttccctcagtgttgattgatgagcttcatggattactcttgtatattggacacctatctgaacttcccagtgttaatataggagcatttgtaaatcaaaaccagattaagctttttccaccgtcatggcatttattacatctccacttggatatacattggctggtgctagaaattctttacatgctgggtgaaaaattgaaacaagttgtatatggtcatcagtttatgaatctggcaagtgacaatttaaccaacatcagcctatttgaagaacattgtgaaactctcctttgtgatttaataagcctgtcactcaacaggtacgacaaggttaggtcttctgaatcattaatgagtgaccagtgtccatgtttatgcattaaagaattatgggttctacttattcatcttctagaccacagaagtaaatggtttgtctcggaatcattttggaactggttgaataaactacttaaaacactgcttgaaaaatcaagtgaccgaagaagatcctctatgcctgtaatccagtccagggatccattaggttttagttggtggattattactcatgtagcatcattttacaagtttgatcgccatggagtaccagatgaaatgagaaaagtggaatcaaattggaactttgtagaagaactgctgaaaaagtccatcagtgttcagggtgtcattctagaagaacaattacgaatgtatcttcactgttgtttgacactttgtgatttctgggagccaaacattgcaattgttaccattttatgggaatattatagtaagaacctgaatagttccttcagtatttcttggcttccttttaaaggccttgctaataccatgaagtcacccttgtctatgcttgaaatggtgaagacttgctgttgcgataaacaagatcaggaactatataaatccagcagtagttatactatttttctttgtattctggcaaaagttgttaaaaaagcaatgaagagcaatggccctcatccttggaaacaagtcaaaggaagaatatattcaaaattccatcaaaaaagaatggaagaactaactgaagttggtctacagaacttttttagcctttttctactgttagcagctgttgcagaggtagaagatgttgcaagtcatgttttagacctcctgaatttcctcaagcctgcttttgtaacgtctcagagagccctcatttggaagggtcacatggccttcctcttgatgtatgcccagaaaaatctggacattggtgttttggctgagaaattttcatgtgctttccgggagaaagcaaaggaattcttggtgtctaagaatgaggaaatggtacagagacagactatctggacccttctttccatatacattgatggtgttcaagaagtgtttgagaccagctattgcttgtatccttcccatgaaaaactgcttaatgatggatttagtatgcttctgcgagcatgtcgagaatctgaacttaggacagtattgagcttcctacaagctgttctggccagaatcaggagtatgcatcaacaattgtgtcaggaacttcaaagggacaatgtggacctatttgtacagtcttcattatcggctaaagagcgccaccttgctgcagttgccagtgcactgtggagacatttcttttcatttttgaagagtcagagaatgtcacaggtagtgcctttctcacaacttgcggatgcagctgcagactttactttgctagcaatggacatgccaagcacagctccatcagattttcagcctcagccagttatatcaattattcaactttttggttgggatgatatcatctgccctcaagttgtagcaagatatttaagtcatgtcctacaaaatagcacattatgtgaagcactttctcattcaggctatgtatcttttcaagccttaaccgtaagatcatggattcgttgtgttttgcaaatgtatattaaaaacctctctgggcctgatgatttgctcatagataaaaatctggaagaggcagttgaaaaagagtacatgaaacagttggtcaaactgacaagattactatttaatctctcagaagtaaagagtattttctcaaaggcccaagttgaatatttatccatctcagaagaccctaaaaaagcacttgttcgattctttgaggctgttggtgtaacttacgggaacgtccagacactttctgataaatctgccatggtcacaaagtccttggaataccttggtgaagtattaaaatatattaagccttatttgggaaaaaaagttttcagtgcagggctgcagctgacttatggaatgatgggaattcttgtgaaatcatgggcacaaatctttgccacttctaaagcccaaaaattactattccggatcatagattgtttactgctgccacatgcagtattacagcaagagaaggaactgcctgcacctatgttgtcagcaattcagaaaagtcttcctttgtatctccagggcatgtgtatcgtgtgttgtcaatctcaaaatccgaatgcctatttgaatcaattgctagggaatgttattgagcagtatattgggcgatttcttccagcttcaccatatgtttcagatcttggacaacatcctgttttgctggcattgagaaacacagccactattccaccaatatcatctctaaagaaatgcattgtgcaagtcataaggaaatcctaccttgagtataaggggtcctcacctcctcctcgcttagcatccattctggccttcatcctccaactcttcaaggaaactaacacagacatttatgaagttgaactactcctccctggcattttaaaatgcttggtgttagtcagtgaaccacaagttaaaaggctggccacagagaacctgcaatacatggtaaaagcctgccaagtggggtcagaagaagaaccttcctcccagctgacttctgtgtttaggcagtttatccaggattatggtatgaggtactattaccaggtttacagcattttagaaacagtagcaacattggaccagcaggttgtcatccacttgatttctacccttactcagtctctgaaggattcagagcagaaatggggccttggcaggaatatagcacaaagggaagcctatagcaaacttttgtctcaccttggacagatgggacaagatgagatgcagagactggaaaatgataatacttaatatattttgtgattcatcctgtctattaattatcagtatctaaagccacattgttccactctaattcctacttttaattaattgtataatgttctgtttaaaataatttctaggattttttttccttttatatctgtaagtaaaaaatgtataaataaggaactgaaggttaattagcaactgtgtaataggatccttggaactttttacaaaactgcattctaccttgatgaaattttagttaaactaaattaaagaaacattttctgagagtgttttctgtgtgtgagtattaagcagatacttcatccatagactttttgttgtagtatgacttcctgcctccctgccttgtgactgatttgtcatgtttattttttatttttacaataatagttatgttttaaaatgatactttttcaaagtacctggctaccctatactgtgtttttggtgggggaaaggtgttgggaaatgtgactgtgtatttgtttttcttttttatatgtgtgaggcaagttaacaaagaaaacccttacaaagcacacttaactgtgttaggaatgagactgaatctggctaaaggattatacagctgatatccaaaaagaagtgtaaacatgccaacagggtttatatttaggttccaagagttgccaaattttttttttattcatcctttcttttggagttgtcattaaaaaaaaaatcatatcaggaggaggaaataaaggagactttaagaggagagtaaataaaaatattctagaaaagtaacaatagtagtggtctgagaagtcagctgatccatcttcttgctctttagccagagcttcttataacttagcattttatccaagaaacctgtttagtttcctttaacaacattgtttctcaacttttgaactcttaactcatccagattgttttaccagttggagcttgtccttgactccttttcctattgagcattatttggttgaaagagctccagtcctagttttgggaacccacctgggtctcaaactcagctttgctttatgatcttgagctagttacgtaacctttctcagtctagtttcttcatggagaagaaaagaagaatccctttactatttgacagtgttgttgtgagaatcaatgggataatgtacgtgaaaatatttggtggcccaaattctgttgggaaatgtacagacacgattagtttattactcgtgcacaattaaggcaaagaacacctctcaattttatctttctctaatgatatgtcccagatgaatgatcccactttccaatgttttcttacatcatgttttctaactttgaagtactttgtttccttcttgggattatagtctttatcatggttctactcttccttcaagaaaacatttcaacccttctctggctcaaggagtatctttaaaaatgaaatactttgcaatcagtattctgtagttaatagcgttctcttaattataatctgtgaaatccaaaggagtgtctgttattttcataacatataaatggtccttctctcaaatcagcttcatcaaagggaataaatgaaagcttaaggctgtttaatagacttcataacactgaatttcttttcatcatgatagtgacttactaaacaaataatgtatggcttggaaagttccagcagtcactaaggagttttatctatttaaggtgatgcctttaaagaggttatttatgtgtatatgtgtttttgtgtgtgtgtgtgtgtgtgtgtgtgtgtgtgtgtgtgtgtgggttttttttttccaaaggagattgataaagatacctttgagaatcttgctccttagagtatgaaaacgtttaaaggataggaaaagctgctactgaatcatgctttaggacatatatctatagatactaagaataactaaaatttgtacttaagaatggtaacaacaaagtgtgtaacaatatgtgtgtgtattatatgttttgtatttatgtataagtaactttttttaaagcttttttttaaattgaaaaaagaagaaaatttatggctcagttccttttttttaaaatcttcactgatccttcacttattactacaatcttgccctctttcattcccagacttggagtcccttatacctgcttctttaatagccactttccctaaataccctgtaatttctgaggctgctgcttctaccagtgtaatacaagccattgtcttttggtcaaatctgagtttgttctcctttctcatgttctttgtcctctctgtgacaattgtattttcttttatcttcccatcatttccacaatggtactgctccttcctacatatacagttgacccttgagcaacacaagtttgaactgtgcaggtccacttatatgtggatctaaaatacagtatttgtgggatgcaaaatctgagtatatggaggaccaactttttgtagaggcaggttcctcagggttaactgcaggacttgagtaagtgcagattatggtatgcacaggggatcctggaaccagtccccagaccaaaaatgaccactgtatcttgagagcaccttcatggtcttctttcctgtccccagtcgtaagatacgacccttcatctgctttgtctcaacattctgttagtggtgaactgataactccagcttttctctctctttttttttttttttttttttttgttgttgttgttgttgttgttttgaggcagagtcttgctctgtcgcccaggctggagtgcagtggcacgatctcggctcactgcaagctctgcctcccgggttcacgccattctcctgcctcagcctcccaagtagctgggactacaggtgcccgccaccacgcccggctaattttttttgtatttttagtagagacggggtttcaccatgttgggctcgatctcctgacctcatgatccgcccgcctcggcctcccaaagtgctgggattacaggcgtgatgagccacctagtgctaattcatctctaaacctctgtagttggagtctcctgaaatctcagactacctcctgctttcattttctacccagggaatacactattgtcctaagttatttagccttgaaatgtcagaggcattttaggttcttcatgtttctcccactttcagttgattaccattacaggatcttaggcttctttttatctttttgcagtgccttgcatccactggttgtttgttttttctgttgggcactacttatttccttacctctggtttcttctcctacagcttatgttctgaaatgtgtattcctaaaatactgttttcctcttgttacttttctgctcgaggacttccagtaacttaccattgtttataaggtgatgttcaaacccttcagtctggagactttctgccacctatctgactgtacctttatcttcaagcttatctctttccaaacaagaggctcatttagcagctttctctgattgccatgttcatcctacactcagcctcattcagtttacagcatggaaactgtataggacctctttcctatagaaattgaagacacttaaataggaagaaaattaaaatatacatttggatacatgagtattccagtcaaataatatctataaaataccagatagagtataaaagacaactgaaggacaacagagtgatgaaaggactttattaggcatttggatttggttatgatttaaatttcaatttaattagaacgtttccatggcaaggaaggaagcatggaggactgtggaaaagtcattcagtattgagttcatttgcattagaggaatttcatagtttaaaacttgtatatctttacctatccttcgtatgttttcttcttaagcatatttgactttttctacctcagcatctgtataagaaaatatttgtgagtcagatgtttgtgggttttccttacctattattattttcttccatgctttacaacacattttttaaactaccttgttcttaaataattacacggacctgcttctgtgtactttcacagaatctttgacagttaaaaattgtatgttatataaaaatttgacaagcttctacagttaggaaaagcctttagaaatctgccttccccaaaccgtatgttatcatagcactcatgtctccccatgtctaaaaggtaaatagatacagaaacctcaccaaaagttgaatcatctgtactaaaccactgcttttttatccagactttttatataaatcttttattttccaaaaaactgatcttttacctacccctcttagagttaaaaatattgcctgtcaggcaagagtatagtatgccaatataaataatatgctttatttggttagaaacagttggtttgggaagctagcaacatagcatattctttacaaatttaataaggtgtattttgatactgtgataacctgcattgtaaattaccaaatggttggaattgaccagtttatacttattaaaatttttgatgtgccaatggctatccaattcattgcatttgtgaattgtgcttttagaaaaattgtggacattttagttttatatacatatttatgagttatttgtgcatatgataaaattatatataataataaaagtatatatgtaactta

SEQ ID NO: 159 - protein, 1243 aa, Homo sapiens
MENCSAASTFLTDSLELELGTEWCKPPYFSCAVDNRGGGKHFSGESYLCSGALKRLILNLDPLPTNFEEDTLEIFGIQWVTETALVNSSRELFHLFRQQLYNLETLLQSSCDFGKVSTLHCKADNIRQQCVLFLHYVKVFIFRYLKVQNAESHVPVHPYEALEAQLPSVLIDELHGLLLYIGHLSELPSVNIGAFVNQNQIKLFPPSWHLLHLHLDIHWLVLEILYMLGEKLKQVVYGHQFMNLASDNLTNISLFEEHCETLLCDLISLSLNRYDKVRSSESLMSDQCPCLCIKELWVLLIHLLDHRSKWFVSESFWNWLNKLLKTLLEKSSDRRRSSMPVIQSRDPLGFSWWIITHVASFYKFDRHGVPDEMRKVESNWNFVEELLKKSISVQGVILEEQLRMYLHCCLTLCDFWEPNIAIVTILWEYYSKNLNSSFSISWLPFKGLANTMKSPLSMLEMVKTCCCDKQDQELYKSSSSYTIFLCILAKVVKKAMKSNGPHPWKQVKGRIYSKFHQKRMEELTEVGLQNFFSLFLLLAAVAEVEDVASHVLDLLNFLKPAFVTSQRALIWKGHMAFLLMYAQKNLDIGVLAEKFSCAFREKAKEFLVSKNEEMVQRQTIWTLLSIYIDGVQEVFETSYCLYPSHEKLLNDGFSMLLRACRESELRTVLSFLQAVLARIRSMHQQLCQELQRDNVDLFVQSSLSAKERHLAAVASALWRHFFSFLKSQRMSQVVPFSQLADAAADFTLLAMDMPSTAPSDFQPQPVISIIQLFGWDDIICPQVVARYLSHVLQNSTLCEALSHSGYVSFQALTVRSWIRCVLQMYIKNLSGPDDLLIDKNLEEAVEKEYMKQLVKLTRLLFNLSEVKSIFSKAQVEYLSISEDPKKALVRFFEAVGVTYGNVQTLSDKSAMVTKSLEYLGEVLKYIKPYLGKKVFSAGLQLTYGMMGILVKSWAQIFATSKAQKLLFRIIDCLLLPHAVLQQEKELPAPMLSAIQKSLPLYLQGMCIVCCQSQNPNAYLNQLLGNVIEQYIGRFLPASPYVSDLGQHPVLLALRNTATIPPISSLKKCIVQVIRKSYLEYKGSSPPPRLASILAFILQLFKETNTDIYEVELLLPGILKCLVLVSEPQVKRLATENLQYMVKACQVGSEEEPSSQLTSVFRQFIQDYGMRYYYQVYSILETVATLDQQVVIHLISTLTQSLKDSEQKWGLGRNIAQREAYSKLLSHLGQMGQDEMQRLENDNT

SEQ ID NO: 160 - DNA, 22 bp, Artificial Sequence
  Artificial sequence
gtctaccaggcattcgcttcat

SEQ ID NO: 161 - DNA, 24 bp, Artificial Sequence
  Artificial sequence
tcagctggaccacagccgcagcgt

SEQ ID NO: 162 - DNA, 21 bp, Artificial Sequence
  Artificial sequence
tcagaaatcctttctcttgac

SEQ ID NO: 163 - DNA, 24 bp, Artificial Sequence
  Artificial sequence
ctagcctctggaatcctttctctt

(19) United States
(12) Patent Application Publication
(10) Pub. No.: US 2012/0128705 A1
(43) Pub. Date: May 24, 2012

(54) C6ORF167 PEPTIDES AND VACCINES CONTAINING THE SAME

(75) Inventors: Yusuke Nakamura, Tokyo (JP); Yataro Daigo, Tokyo (JP); Takuya Tsunoda, Kanagawa (JP); Ryuji Ohsawa, Kanagawa (JP); Sachiko Yoshimura, Kanagawa (JP); Tomohisa Watanabe, Kanagawa (JP)

(73) Assignee: OncoTherapy Science, Inc., Kanagawa (JP)

(21) Appl. No.: 13/260,900

(22) PCT Filed: Mar. 31, 2010
(86) PCT No.: PCT/JP2010/002352

§ 371 Date: Dec. 21, 2011

Related U.S. Application Data
(60) Provisional application No. 61/211,700, filed on Apr. 1, 2009.

(51) Int. Cl. A61K 38/08; C07H 21/04; A61K 31/711; A61K 39/00; C12N 15/63; A61P 35/00; C12N 5/02; C12N 5/00; C12Q 1/68; G01N 33/53; C07K 7/06; C07K 16/00

(52) U.S. Cl. 424/185.1; 530/328; 536/23.1; 514/19.3; 514/44 R; 530/389.1; 435/375; 435/373; 435/325; 435/6.14; 435/7.1; 435/320.1

(57) ABSTRACT

Peptide vaccines against cancer are described herein. In particular, epitope peptides derived from the C6orf167 gene that elicit CTLs are provided. Antigen-presenting cells and isolated CTLs that target such peptides, as well as methods for inducing the antigen-presenting cell, or CTL are also provided. The present invention further provides pharmaceutical compositions containing peptides derived from C6orf167 or polynucleotides encoding the polypeptides as active ingredients. Furthermore, the present invention provides methods for the treatment and/or prophylaxis of (i.e., preventing) cancers (tumors), and/or the prevention of postoperative recurrence thereof, as well as methods for inducing CTLs, methods for inducing anti-tumor immunity, using the peptides derived from C6orf167, polynucleotides encoding the peptides, or antigen-presenting cells presenting the peptides, or the pharmaceutical compositions of the present invention.

The present application claims the benefit of U.S. Provisional Applications No. 61/211,700, filed on Apr. 1, 2009, the entire contents of which are incorporated by reference herein.

### TECHNICAL FIELD

The present invention relates to the field of biological science, more specifically to the field of cancer therapy. In particular, the present invention relates to novel peptides that are effective as cancer vaccines, drugs for treating and preventing tumors, and methods for diagnosing tumors.

### BACKGROUND ART

It has been demonstrated that CD8 positive cytotoxic T lymphocytes (CTLs) recognize epitope peptides derived from the tumor-associated antigens (TAAs) found on the major histocompatibility complex (MHC) class I molecule, and then kill the tumor cells. Since the discovery of the melanoma antigen (MAGE) family as the first example of TAAs, many other TAAs have been discovered, primarily through immunological approaches (NPL 1/Boon T, Int J Cancer 1993 May 8, 54(2): 177-80; NPL 2/Boon T & van der Bruggen P, J Exp Med 1996 Mar. 1, 183(3): 725-9). Some of these TAAs are in currently undergoing clinical development as immunotherapeutic targets.

Identification of new TAAs capable of inducing potent and specific anti-tumor immune responses warrants further development and clinical application of peptide vaccination strategies for various types of cancer (NPL 3/Harris C C, J Natl Cancer Inst 1996 Oct. 16, 88(20): 1442-55; NPL 4/Butterfield L H et al., Cancer Res 1999 Jul. 1, 59(13): 3134-42; NPL 5/Vissers J L et al., Cancer Res 1999 Nov. 1, 59(21): 5554-9; NPL 6/van der Burg S H et al., J Immunol 1996 May 1, 156(9): 3308-14; NPL 7/Tanaka F et al., Cancer Res 1997 Oct. 15, 57(20): 4465-8; NPL 8/Fujie T et al., Int J Cancer 1999 Jan. 18, 80(2): 169-72; NPL 9/Kikuchi M et al., Int J Cancer 1999 May 5, 81(3): 459-66; NPL 10/Oiso M et al., Int J Cancer 1999 May 5, 81(3): 387-94). To date, there have been several reports of clinical trials using these tumor-associated antigen derived peptides. Unfortunately, only a low objective response rate has been observed in these cancer vaccine trials so far (NPL 11/Belli F et al., J Clin Oncol 2002 Oct. 15, 20(20): 4169-80; NPL 12/Coulie P G et al., Immunol Rev 2002 October, 188: 33-42; NPL 13/Rosenberg S A et al., Nat Med 2004 September, 10(9): 909-15). Therefore, identification of novel TAAs, which is expected to be useful as an immunotherapeutic target, are still required.

To that end, a number of up-regulated genes have been identified in small cell lung cancers (SCLCs) (PTL 1/WO2007/013665) and esophageal cancers (PTL 2/WO2007/013671) through analyses of gene-expression profiles using genome-wide cDNA microarrays. These genes have been amply investigated with the hopes of identifying good candidates as immunotherapeutic targets from among them. In order to target cancer cells specifically in immunotherapy, preferred TAAs should be expressed primarily by cancer cells, with limited or no expression by normal healthy tissues.

Chromosome 6 open reading frame 167 (C6orf167) was identified via cDNA library screening of the Mammalian Gene Collection (NPL 14/MGC Program Team, Proc Natl Acad Sci USA. 2002 Dec. 24; 99 (26):16899-903). C6orf167 was one of the up-regulated genes identified in the aforementioned analyses. However, its availability for diagnosis and/or therapy in cancers has yet to be confirmed.

The present invention addresses the need in the art for improved cancer diagnosis and therapy by providing a novel cancer marker, C6orf167, and a novel therapy for cancer that utilizes an antigenic peptide, particularly antigenic peptides and cancer vaccines that target C6orf167.

### CITATION LIST

### Patent Literature

[PTL 1] WO2007/013665
[PTL 2] WO2007/013671

### Non Patent Literature

[NPL 1] Boon T, Int J Cancer 1993 May 8, 54(2): 177-80
[NPL 2] Boon T & van der Bruggen P, J Exp Med 1996 Mar. 1, 183(3): 725-9
[NPL 3] Harris CC, J Natl Cancer Inst 1996 Oct. 16, 88(20): 1442-55
[NPL 4] Butterfield L H et al., Cancer Res 1999 Jul. 1, 59(13): 3134-42
[NPL 5] Vissers J L et al., Cancer Res 1999 Nov. 1, 59(21): 5554-9
[NPL 6] van der Burg S H et al., J Immunol 1996 May 1, 156(9): 3308-14
[NPL 7] Tanaka F et al., Cancer Res 1997 Oct. 15, 57(20): 4465-8
[NPL 8] Fujie T et al., Int J Cancer 1999 Jan. 18, 80(2): 169-72
[NPL 9] Kikuchi M et al., Int J Cancer 1999 May 5, 81(3): 459-66
[NPL 10] Oiso M et al., Int J Cancer 1999 May 5, 81(3): 387-94
[NPL 11] Belli F et al., J Clin Oncol 2002 Oct. 15, 20(20): 4169-80
[NPL 12] Coulie P G et al., Immunol Rev 2002 October, 188: 33-42
[NPL 13] Rosenberg S A et al., Nat Med 2004 September, 10(9): 909-15
[NPL 14] MGC Program Team, Proc Natl Acad Sci USA. 2002 Dec. 24; 99 (26):16899-903

### SUMMARY OF INVENTION

The present invention relates to novel peptides suited to cancer therapy and methods for detecting or diagnosing cancer.

The present invention is based, at least in part, on the discovery of novel peptides that may serve as targets of immunotherapy. Because TAAs are generally perceived by the immune system as “self” and therefore often have no innate immunogenicity, the discovery of appropriate targets is of extreme importance. Through the present invention, C6orf167 (SEQ ID NO: 159 encoded by the gene of GenBank Accession No. NM_{—}198468.2 (SEQ ID NO: 158)) is demonstrated to be specifically over-expressed in cancer cells, including bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor, and testicular tumor, but not limited thereto. Thus, the present invention focuses on C6orf167 as an appropriate cancer marker and candidate for the target of immunotherapy.

The present invention further relates to the identification of specific epitope peptides of the gene product of C6orf167 that possess the ability to induce CTLs specific to C6orf167. As discussed in detail below, peripheral blood mononuclear cells (PBMCs) obtained from a healthy donor were stimulated using HLA-A*2402 or HLA-A*0201 binding candidate peptides derived from C6orf167. CTL lines were then established with specific cytotoxicity against the HLA-A24 or HLA-A2 positive target cells pulsed with each of candidate peptides. These results demonstrate that these peptides are HLA-A24 or HLA-A2 restricted epitope peptides that can induce potent and specific immune responses against cells expressing C6orf167. Further, the results indicate that C6orf167 is strongly immunogenic and that the epitopes thereof are effective targets for tumor immunotherapy.

Accordingly, it is an object of the present invention to provide isolated peptides that bind to HLA antigen, particularly those that include C6orf167 (SEQ ID NO: 158) or an immunogenic fragment. These peptides are expected to have CTL inducibility and, thus, can be used to induce CTL ex vivo or to be administered to a subject for inducing immune responses against cancers, such as bladder cancer, cervical cancer, cholangio-cellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor, but not limited thereto. Preferred peptides are nonapeptides or decapeptides, and more preferably those having an amino acid sequence selected from among SEQ ID NOs: 1 to 61 and 63 to 151. Of these, the peptides having an amino sequence selected from among SEQ ID NOs: 2, 4, 7, 8, 9, 14, 16, 18, 22, 25, 26, 30, 33, 34, 35, 36, 38, 39, 41, 43, 44, 45, 47, 48, 49, 53, 65, 66, 76, 79, 84, 101, 110, 111, 112, 113, 114, 117, 118, 121, 122, 123, and 124 are most preferred.

The peptides of the present invention encompass those wherein one, two or more amino acids are substituted or added, so long as the resulting modified peptides retain the original CTL inducibility.

The present invention also provides isolated polynucleotides encoding any one of peptides of the present invention. These polynucleotides can be used to induce APCs with CTL inducibility or can be administered to a subject for inducing immune responses against cancers much like the present peptides.

When administered to a subject, the present peptides are preferably presented on the surface of APCs so as to induce CTLs targeting the respective peptides. Accordingly, it is further object of the present invention to provide compositions that induce CTL, such compositions including one or more peptides of the present invention, or polynucleotides encoding such peptides. The present invention further contemplates pharmaceutical compositions including one or more peptides of present invention, or one or more polynucleotide encoding such peptides formulated for the treatment and/or prophylaxis of cancers as well as the prevention of postoperative recurrence thereof, such cancers including, but not limited to, bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor.

It is a further object of the present invention to provide methods for inducing antigen-presenting cells (APCs) with CTL inducibility, such methods including the step of contacting an APC with one or more peptides of the present invention, or the step of introducing a polynucleotide encoding any one of the peptide of the present invention into an APC.

The present invention also provides methods for inducing CTL that include the step of co-culturing CD8 positive T cells with APCs presenting on its surface a complex of an HLA antigen and one or more peptides of the present invention, the step of co-culturing CD8 positive T cells with exosomes presenting on its surface a complex of an HLA antigen and one or more peptides of the present invention, or the step of introducing a gene that includes one or more polynucleotides coding for a T cell receptor (TCR) subunit polypeptide that binds to a peptide of the present invention.

It is yet another object of the present invention to provide isolated APCs that present on its surface a complex of an HLA antigen and a peptide of the present invention. The present invention further provides isolated CTLs that target peptides of the present invention. These APCs and CTLs may be used for cancer immunotherapy.

It is another object of the present invention to provide methods for inducing an immune response against cancer in a subject in need thereof, such methods including the step of administering to the subject a composition including a peptide of the present invention or a polynucleotide encoding such a peptide.

It is yet another object of the present invention to provide methods for diagnosing cancer in a subject in need thereof, such methods including the steps of (a) determining the expression level of the C6orf167 gene in the subject-derived biological sample, and (b) relating an increase in the expression level determined in step (a) as compared to a normal control level of the gene to the presence of cancer in the subject. Preferably, the expression level of the C6orf167 gene is determined by detecting the mRNA or protein of C6orf167, or a biological activity of the C6orf167 protein.

It is yet a further object of the present invention to provide a kit for use in diagnosing cancer, such a kit including a reagent for detecting the C6orf167 mRNA, a reagent for detecting the C6orf167 protein, or a reagent for detecting the biological activity of the C6orf167 protein.

The applicability of the present invention extends to any of a number of diseases relating to or arising from C6orf167 over-expression, such as cancer, examples of which include, but are not limited to, bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor, but not limited thereto.

In addition to the above, other objects and features of the invention will become more fully apparent when the following detailed description is read in conjunction with the accompanying figures and examples. However, it is to be understood that both the foregoing summary of the invention and the following detailed description are of exemplified embodiments, and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### BRIEF DESCRIPTION OF DRAWINGS

Various aspects and applications of the present invention will become apparent to the skilled artisan upon consideration of the brief description of the figures and the detailed description of the present invention and its preferred embodiments that follows.

FIG. 1a-j is composed of a series of photographs, (a)-(j), depicting the results of IFN-gamma ELISPOT assays on CTLs that were induced with peptides derived from C6orf167. The CTLs in the following well numbers showed potent IFN-gamma production as compared with the control: well #1 stimulated with C6orf167-A24-9-179 (SEQ ID NO: 2) (a), #1 and #3 with C6orf167-A24-9-404 (SEQ ID NO: 4) (b), #4 with C6orf167-A24-9-236 (SEQ ID NO: 7) (c), #1 and #7 with C6orf167-A24-9-480 (SEQ ID NO: 8) (d), #7 with C6orf167-A24-9-1170 (SEQ ID NO: 9) (e), #5 with C6orf167-A24-9-9 (SEQ ID NO: 14) (f), #3 and #4 with C6orf167-A24-9-530 (SEQ ID NO: 16) (g), #5 with C6orf167-A24-9-315 (SEQ ID NO: 18) (h), #3 with C6orf167-A24-9-132 (SEQ ID NO: 22) (i), and #1 and #7 with C6orf167-A24-9-851 (SEQ ID NO: 25) (j). The cells in the wells denoted with a rectangular box were expanded to establish CTL lines. In the figures, “+” indicates the IFN-gamma production against target cells pulsed with the appropriate peptide, and “−” indicates the IFN-gamma production against target cells not pulsed with any peptides.

FIG. 1k-t is composed of a series of photographs, (k)-(t), depicting the results of IFN-gamma ELISPOT assays on CTLs that were induced with peptides derived from C6orf167. The CTLs in the following well numbers showed potent IFN-gamma production as compared with the control: #3 and #6 with C6orf167-A24-9-55 (SEQ ID NO: 26) (k), #1 and #2 with C6orf167-A24-9-220 (SEQ ID NO: 30) (l), #4 and #8 with C6orf167-A24-10-626 (SEQ ID NO: 33) (m), #1 with C6orf167-A24-10-429 (SEQ ID NO: 34) (n), #1 and #5 with C6orf167-A24-10-917 (SEQ ID NO: 35) (o), #4 and #5 with C6orf167-A24-10-474 (SEQ ID NO: 36) (p), #4 with C6orf167-A24-10-254 (SEQ ID NO: 38) (q), #2 with C6orf167-A24-10-194 (SEQ ID NO: 39) (r), #7 with C6orf167-A24-10-956 (SEQ ID NO: 41) (s), #3 with C6orf167-A24-10-511 (SEQ ID NO: 43) (t). The cells in the wells denoted with a rectangular box were expanded to establish CTL lines. In the figures, “+” indicates the IFN-gamma production against target cells pulsed with the appropriate peptide, and “−” indicates the IFN-gamma production against target cells not pulsed with any peptides.

FIG. 1u-z is composed of a series of photographs, (u)-(z), depicting the results of IFN-gamma ELISPOT assays on CTLs that were induced with peptides derived from C6orf167. The CTLs in the following well numbers showed potent IFN-gamma production as compared with the control: #3 with C6orf167-A24-10-315 (SEQ ID NO: 44) (u), #2 with C6orf167-A24-10-598 (SEQ ID NO: 45) (v), #1 and #3 with C6orf167-A24-10-966 (SEQ ID NO: 47) (w), #7 with C6orf167-A24-10-66 (SEQ ID NO: 48) (x), #3 with C6orf167-A24-10-914 (SEQ ID NO: 49) (y) and #2 with C6orf167-A24-10-851 (SEQ ID NO: 53) (z). The cells in the wells denoted with a rectangular box were expanded to establish CTL lines. In the figures, “+” indicates the IFN-gamma production against target cells pulsed with the appropriate peptide, and “−” indicates the IFN-gamma production against target cells not pulsed with any peptides.

FIG. 2a-h is composed of a series of line graphs, (a)-(h), depicting the results of an IFN-gamma ELISA assay demonstrating the IFN-gamma production of CTL lines stimulated with SEQ ID NO: 2 (a), SEQ ID NO: 4 (b), SEQ ID NO: 7 (c), SEQ ID NO: 8 (d), SEQ ID NO: 9 (e), SEQ ID NO: 16 (f), SEQ ID NO: 22 (g), and SEQ ID NO: 25 (h). The results demonstrate that CTL lines established by stimulation with each peptide showed potent IFN-gamma production as compared with the control. In the figures, “+” indicates the IFN-gamma production against target cells pulsed with the appropriate peptide and “−” indicates the IFN-gamma production against target cells not pulsed with any peptides.

FIG. 2i-n is composed of a series of line graphs, (i)-(n), depicting the results of an IFN-gamma ELISA assay demonstrating the IFN-gamma production of CTL lines stimulated with SEQ ID NO: 26 (i), SEQ ID NO: 30 (j), SEQ ID NO: 33 (k), SEQ ID NO: 35 (l), SEQ ID NO: 44 (m) and SEQ ID NO: 49 (n). The results demonstrate that CTL lines established by stimulation with each peptide showed potent IFN-gamma production as compared with the control. In the figures, “+” indicates the IFN-gamma production against target cells pulsed with the appropriate peptide and “−” indicates the IFN-gamma production against target cells not pulsed with any peptides.

FIG. 3 is composed of a series of line graphs, (a) to (f), depicting the IFN-gamma production of the CTL clones established by limiting dilution from the CTL lines stimulated with SEQ ID NO: 2 (a), SEQ ID NO: 4 (b), SEQ ID NO: 7 (c), SEQ ID NO: 9 (d), SEQ ID NO: 16 (e) and SEQ ID NO: 30 (f). The results demonstrate that the CTL clones established by stimulation with SEQ ID NO: 2 (a), SEQ ID NO: 4 (b), SEQ ID NO: 7 (c), SEQ ID NO: 9 (d), SEQ ID NO: 16 (e) and SEQ ID NO: 30 (f) showed potent IFN-gamma production as compared with the control. In the figure, “+” indicates the IFN-gamma production against target cells pulsed with SEQ ID NO: 2 (a), SEQ ID NO: 4 (b), SEQ ID NO: 7 (c), SEQ ID NO: 9 (d), SEQ ID NO: 16 (e) and SEQ ID NO: 30 (f) and “−” indicates the IFN-gamma production against target cells not pulsed with any peptides.

FIG. 4 is composed of a series of line graphs, (a) to (d), depicting the specific CTL activity against the target cells that exogenously express C6orf167 and HLA-A*2402. COS7 cells transfected with HLA-A*2402 or with the full length of the C6orf167 gene were prepared as controls. The CTL lines established with C6orf167-A24-9-179 (SEQ ID NO: 2) (a), C6orf167-A24-9-236 (SEQ ID NO: 7) (b), C6orf167-A24-9-1170 (SEQ ID NO: 9) (c) and C6orf167-A24-10-626 (SEQ ID NO: 33) (d) showed specific CTL activity against COS7 cells transfected with both C6orf167 and HLA-A*2402 (black lozenge). On the other hand, no significant specific CTL activity was detected against target cells expressing either HLA-A*2402 (triangle) or C6orf167 (circle).

FIG. 5 depicts the analytical results of the C6orf167 expression in tumor tissues, cell lines and normal tissue. Part A depicts the expression of C6orf167 in 15 clinical lung cancers samples [lung adenocarcinoma (ADC), lung squamous cell carcinoma (SCC) and lung small cell lung carcinoma (SCLC); top], 10 clinical esophageal cancer samples (upper panels), 15 lung cancers cell lines and 10 esophageal cancer cell lines (lower panels) detected by semiquantitative RT-PCR analysis. Panel B depicts the Northern blot analysis of the C6orf167 transcript in 16 normal human tissues. Positive signals were observed only in testis and small intestine.

FIG. 6a-f is composed of a series of photographs, (a) to (f), depicting the results of IFN-gamma ELISPOT assay on CTLs that were induced with peptides derived from C6orf167. The CTLs in the following well numbers showed potent IFN-gamma production as compared with the control: well #4 with C6orf167-A02-9-855 (SEQ ID NO: 65) (a), #6 with C6orf167-A02-9-131 (SEQ ID NO: 66) (b), #4 with C6orf167-A02-9-887 (SEQ ID NO: 76) (c), #6 with C6orf167-A02-9-261 (SEQ ID NO: 79) (d), #7 with C6orf167-A02-9-484 (SEQ ID NO: 84) (e), and #1, #3 and #6 with C6orf167-A02-10-535 (SEQ ID NO: 101) (f). The cells in the wells denoted with a rectangular box were expanded to establish CTL lines. In contrast, as is typical of negative data, no specific IFN-gamma production was observed from the CTL stimulated with C6orf167-A02-9-918 (SEQ ID NO: 62) (s). In the figures, “+” indicates the IFN-gamma production against target cells pulsed with the appropriate peptide, and “−” indicates the IFN-gamma production against target cells not pulsed with any peptides.

FIG. 6g-l is composed of a series of photographs, (g) to (l), depicting the results of IFN-gamma ELISPOT assay on CTLs that were induced with peptides derived from C6orf167. The CTLs in the following well numbers showed potent IFN-gamma production as compared with the control: #1 with C6orf167-A02-10-527 (SEQ ID NO: 110) (g), #3 with C6orf167-A02-10-10 (SEQ ID NO: 111) (h), #5 with C6orf167-A02-10-577 (SEQ ID NO: 112) (i), #5 and #7 with C6orf167-A02-10-128 (SEQ ID NO: 113) (j), #4 with C6orf167-A02-10-622 (SEQ ID NO: 114) (k), and #1 with C6orf167-A02-10-47 (SEQ ID NO: 116) (l). The cells in the wells denoted with a rectangular box were expanded to establish CTL lines. In contrast, as is typical of negative data, no specific IFN-gamma production was observed from the CTL stimulated with C6orf167-A02-9-918 (SEQ ID NO: 62) (s). In the figures, “+” indicates the IFN-gamma production against target cells pulsed with the appropriate peptide, and “−” indicates the IFN-gamma production against target cells not pulsed with any peptides.

FIG. 6m-r is composed of a series of photographs, (m) to (r), depicting the results of IFN-gamma ELISPOT assay on CTLs that were induced with peptides derived from C6orf167. The CTLs in the following well numbers showed potent IFN-gamma production as compared with the control:#1 with C6orf167-A02-10-219 (SEQ ID NO: 117) (m), #3 with C6orf167-A02-10-1155 (SEQ ID NO: 118) (n), #7 with C6orf167-A02-10-606 (SEQ ID NO: 121) (o), #6 with C6orf167-A02-10-290 (SEQ ID NO: 122) (p), #6 with C6orf167-A02-10-262 (SEQ ID NO: 123) (q) and #8 with C6orf167-A02-10-965 (SEQ ID NO: 124) (r). The cells in the wells denoted with a rectangular box were expanded to establish CTL lines. In contrast, as is typical of negative data, no specific IFN-gamma production was observed from the CTL stimulated with C6orf167-A02-9-918 (SEQ ID NO: 62) (s). In the figures, “+” indicates the IFN-gamma production against target cells pulsed with the appropriate peptide, and “−” indicates the IFN-gamma production against target cells not pulsed with any peptides.

FIG. 6s is composed of a photograph, depicting the results of IFN-gamma ELISPOT assay on CTLs that were induced with peptides derived from C6orf167. As is typical of negative data, no specific IFN-gamma production was observed from the CTL stimulated with C6orf167-A02-9-918 (SEQ ID NO: 62) (s). In the figures, “+” indicates the IFN-gamma production against target cells pulsed with the appropriate peptide, and “−” indicates the IFN-gamma production against target cells not pulsed with any peptides.

FIG. 7a-d is composed of a series of line graphs, (a) to (d), depicting the IFN-gamma production of the CTL lines stimulated with C6orf167-A02-9-855 (SEQ ID NO: 65) (a), C6orf167-A02-9-131 (SEQ ID NO: 66) (b), C6orf167-A02-9-887 (SEQ ID NO: 76) (c), and C6orf167-A02-9-261 (SEQ ID NO: 79) (d), detected by IFN-gamma ELISA assay. The results demonstrate that CTL lines established by stimulation with each peptide showed potent IFN-gamma production as compared with the control. In the figures, “+” indicates the IFN-gamma production against target cells pulsed with the appropriate peptide, and “−” indicates the IFN-gamma production against target cells not pulsed with any peptides.

FIG. 7e-j is composed of a series of line graphs, (e) to (j), depicting the IFN-gamma production of the CTL lines stimulated with C6orf167-A02-9-484 (SEQ ID NO: 84) (e), C6orf167-A02-10-535 (SEQ ID NO: 101) (f), C6orf167-A02-10-527 (SEQ ID NO: 110) (g), C6orf167-A02-10-10 (SEQ ID NO: 111) (h), C6orf167-A02-10-577 (SEQ ID NO: 112) (i), and C6orf167-A02-10-128 (SEQ ID NO: 113) (j) detected by IFN-gamma ELISA assay. The results demonstrate that CTL lines established by stimulation with each peptide showed potent IFN-gamma production as compared with the control. In the figures, “+” indicates the IFN-gamma production against target cells pulsed with the appropriate peptide, and “−” indicates the IFN-gamma production against target cells not pulsed with any peptides.

FIG. 7k-n is composed of a series of line graphs, (k) to (n), depicting the IFN-gamma production of the CTL lines stimulated with C6orf167-A02-10-622 (SEQ ID NO: 114) (k), C6orf167-A02-10-219 (SEQ ID NO: 117) (l), C6orf167-A02-10-290 (SEQ ID NO: 122) (m) and C6orf167-A02-10-262 (SEQ ID NO: 123) (n) detected by IFN-gamma ELISA assay. The results demonstrate that CTL lines established by stimulation with each peptide showed potent IFN-gamma production as compared with the control. In the figures, “+” indicates the IFN-gamma production against target cells pulsed with the appropriate peptide, and “−” indicates the IFN-gamma production against target cells not pulsed with any peptides.

FIG. 8a-f is composed of a series of line graphs, (a) to (f), depicting the IFN-gamma production of the CTL clones established by limiting dilution from the CTL lines stimulated with C6orf167-A02-9-855 (SEQ ID NO: 65) (a), C6orf167-A02-9-131 (SEQ ID NO: 66) (b), C6orf167-A02-9-887 (SEQ ID NO: 76) (c), C6orf167-A02-9-261 (SEQ ID NO: 79) (d), C6orf167-A02-9-484 (SEQ ID NO: 84) (e), and C6orf167-A02-10-535 (SEQ ID NO: 101) (f). The results demonstrate that the CTL clones established by stimulation with each peptide showed potent IFN-gamma production as compared with the control. In the figure, “+” indicates the IFN-gamma production against target cells pulsed with the appropriate peptide and “−” indicates the IFN-gamma production against target cells not pulsed with any peptides.

FIG. 8g-j is composed of a series of line graphs, (g) to (j), depicting the IFN-gamma production of the CTL clones established by limiting dilution from the CTL lines stimulated with C6orf167-A02-10-527 (SEQ ID NO: 110) (g), C6orf167-A02-10-10 (SEQ ID NO: 111) (h), C6orf167-A02-10-128 (SEQ ID NO: 113) (i) and C6orf167-A02-10-622 (SEQ ID NO: 114) (j). The results demonstrate that the CTL clones established by stimulation with each peptide showed potent IFN-gamma production as compared with the control. In the figure, “+” indicates the IFN-gamma production against target cells pulsed with the appropriate peptide and “−” indicates the IFN-gamma production against target cells not pulsed with any peptides.

FIG. 9 is composed of a line graphs (a) and (b) depicting the specific CTL activity against the target cells that exogenously express 6orf167 and HLA-A*0201. COS7 cells transfected with HLA-A*0201 or the full length C6orf167 gene were prepared as the controls. The CTL line established with C6orf167-A02-9-261 (SEQ ID NO: 79) (a) and the CTL clone established with C6orf167-A02-10-622 (SEQ ID NO: 114) (b) showed specific CTL activity against COS7 cells transfected with both C6orf167 and HLA-A*0201 (black lozenge). On the other hand, no significant specific CTL activity was detected against target cells expressing either HLA-A*0201 (triangle) or C6orf167 (circle).

### DESCRIPTION OF EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

### I. Definitions

The words “a”, “an”, and “the” as used herein mean “at least one” unless otherwise specifically indicated.

The terms “polypeptide”, “peptide” and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

The term “amino acid” as used herein refers to naturally occurring and synthetic amino acids, as well as amino acid analogs and amino acid mimetics that similarly function to the naturally occurring amino acids. Naturally occurring amino acids are those encoded by the genetic code, as well as those modified after translation in cells (e.g., hydroxyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids may be referred to herein by their commonly known three letter symbols or the one-letter symbols recommended by the IUPAC-IUB Biochemical Nomenclature Commission.

The terms “gene”, “polynucleotide”, “oligonucleotide”, “nucleotide” and “nucleic acid” are used interchangeably herein and, unless otherwise specifically indicated, are referred to by their commonly accepted single-letter codes.

The term “composition” as used herein is intended to encompass a product including the specified ingredients in the specified amounts, as well as any product which results, directly or indirectly, from combination of the specified ingredients in the specified amounts. Such term in relation to pharmaceutical composition, is intended to encompass a product including the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically or physiologically acceptable carrier. The phrase “pharmaceutically acceptable carrier” or “physiologically acceptable carrier”, as used herein, means a pharmaceutically or physiologically acceptable material, composition, substance or vehicle, including, but are not limited to, a liquid or solid filler, diluent, excipient, solvent or encapsulating material, involved in carrying or transporting the subject scaffolded polypharmacophores from one organ, or portion of the body, to another organ, or portion of the body.

Unless otherwise defined, the term “cancer” refers to the cancers or tumors that over-express the C6orf167 gene, examples of which include, but are not limited to, bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor.

Unless otherwise defined, the terms “cytotoxic T lymphocyte”, “cytotoxic T cell” and “CTL” are used interchangeably herein and unless otherwise specifically indicated, refer to a sub-group of T lymphocytes that are capable of recognizing non-self cells (e.g., tumor/cancer cells, virus-infected cells) and inducing the death of such cells.

Unless otherwise defined, the terms “HLA-A24” refers to the HLA-A24 type containing the subtypes such as HLA-A*2402.

Unless otherwise defined, the term “HLA-A2”, as used herein, representatively refers to the subtypes such as HLA-A*0201 and HLA-A*0206.

Unless otherwise defined, the term “kit” as used herein, is used in reference to a combination of reagents and other materials. It is contemplated herein that the kit may include microarray, chip, marker, and so on. It is not intended that the term “kit” be limited to a particular combination of reagents and/or materials.

To the extent that the methods and compositions of the present invention find utility in the context of the “treatment” of cancer, a treatment is deemed “efficacious” if it leads to clinical benefit such as, reduction in expression of C6orf167 gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

To the extent that the methods and compositions of the present invention find utility in the context of the “prevention” and “prophylaxis” of cancer, such terms are interchangeably used herein to refer to any activity that reduces the burden of mortality or morbidity from disease. Prevention and prophylaxis can occur “at primary, secondary and tertiary prevention levels.” While primary prevention and prophylaxis avoid the development of a disease, secondary and tertiary levels of prevention and prophylaxis encompass activities aimed at the prevention and prophylaxis of the progression of a disease and the emergence of symptoms as well as reducing the negative impact of an already established disease by restoring function and reducing disease-related complications. Alternatively, prevention and prophylaxis can include a wide range of prophylactic therapies aimed at alleviating the severity of the particular disorder, e.g. reducing the proliferation and metastasis of tumors.

In the context of the present invention, the treatment and/or prophylaxis of cancer and/or the prevention of postoperative recurrence thereof include any of the following steps, such as the surgical removal of cancer cells, the inhibition of the growth of cancerous cells, the involution or regression of a tumor, the induction of remission and suppression of occurrence of cancer, the tumor regression, and the reduction or inhibition of metastasis. Effective treatment and/or the prophylaxis of cancer decreases mortality and improves the prognosis of individuals having cancer, decreases the levels of tumor markers in the blood, and alleviates detectable symptoms accompanying cancer. For example, reduction or improvement of symptoms constitutes effectively treating and/or the prophylaxis include 10%, 20%, 30% or more reduction, or stable disease.

In the context of the present invention, the term “antibody” refers to immunoglobulins and fragments thereof that are specifically reactive to a designated protein or peptide thereof. An antibody can include human antibodies, primatized antibodies, chimeric antibodies, bispecific antibodies, humanized antibodies, antibodies fused to other proteins or radiolabels, and antibody fragments. Furthermore, an antibody herein is used in the broadest sense and specifically covers intact monoclonal antibodies, polyclonal antibodies, multispecific antibodies (e.g., bispecific antibodies) formed from at least two intact antibodies, and antibody fragments so long as they exhibit the desired biological activity. An “antibody” indicates all classes (e.g., IgA, IgD, IgE, IgG and IgM).

Unless otherwise defined, all technical and scientific terms used herein have the same meaning as commonly understood by one of ordinary skill in the art to which this invention belongs.

### II. Peptides

To demonstrate that peptides derived from C6orf167 function as an antigen recognized by CTLs, peptides derived from C6orf167 (SEQ ID NO: 159) were analyzed to determine whether they were antigen epitopes restricted by HLA-A24 or A2 which are commonly encountered HLA alleles (Date Y et al., Tissue Antigens 47: 93-101, 1996; Kondo A et al., J Immunol 155: 4307-12, 1995; Kubo R T et al., J Immunol 152: 3913-24, 1994).

Candidates of HLA-A24 binding peptides derived from C6orf167 were identified based on their binding affinities to HLA-A24. The candidate peptide is the following peptides:

C6orf167-A24-9-848 (SEQ ID NO: 1), C6orf167-A24-9-179 (SEQ ID NO: 2), C6orf167-A24-9-641 (SEQ ID NO: 3), C6orf167-A24-9-404 (SEQ ID NO: 4), C6orf167-A24-9-1171 (SEQ ID NO: 5), C6orf167-A24-9-1219(SEQ ID NO: 6), C6orf167-A24-9-236 (SEQ ID NO: 7), C6orf167-A24-9-480 (SEQ ID NO: 8), C6orf167-A24-9-1170 (SEQ ID NO: 9), C6orf167-A24-9-580(SEQ ID NO: 10), C6orf167-A24-9-203 (SEQ ID NO: 11), C6orf167-A24-9-254 (SEQ ID NO: 12), C6orf167-A24-9-158 (SEQ ID NO: 13), C6orf167-A24-9-9 (SEQ ID NO: 14), C6orf167-A24-9-561 (SEQ ID NO: 15), C6orf167-A24-9-530 (SEQ ID NO: 16), C6orf167-A24-9-966 (SEQ ID NO: 17), C6orf167-A24-9-315 (SEQ ID NO: 18), C6orf167-A24-9-92 (SEQ ID NO: 19), C6orf167-A24-9-95 (SEQ ID NO: 20), C6orf167-A24-9-786 (SEQ ID NO: 21), C6orf167-A24-9-132 (SEQ ID NO: 22), C6orf167-A24-9-598 (SEQ ID NO: 23), C6orf167-A24-9-827 (SEQ ID NO: 24), C6orf167-A24-9-851 (SEQ ID NO: 25), C6orf167-A24-9-55 (SEQ ID NO: 26), C6orf167-A24-9-626 (SEQ ID NO: 27), C6orf167-A24-9-908 (SEQ ID NO: 28), C6orf167-A24-9-550 (SEQ ID NO: 29), C6orf167-A24-9-220 (SEQ ID NO: 30), C6orf167-A24-9-437 (SEQ ID NO: 31), C6orf167-A24-10-1170 (SEQ ID NO: 32), C6orf167-A24-10-626 (SEQ ID NO: 33), C6orf167-A24-10-429 (SEQ ID NO: 34), C6orf167-A24-10-917 (SEQ ID NO: 35), C6orf167-A24-10-474 (SEQ ID NO: 36), C6orf167-A24-10-514 (SEQ ID NO: 37), C6orf167-A24-10-254 (SEQ ID NO: 38), C6orf167-A24-10-194 (SEQ ID NO: 39), C6orf167-A24-10-240 (SEQ ID NO: 40), C6orf167-A24-10-956 (SEQ ID NO: 41), C6orf167-A24-10-786 (SEQ ID NO: 42), C6orf167-A24-10-511 (SEQ ID NO: 43), C6orf167-A24-10-315 (SEQ ID NO: 44), C6orf167-A24-10-598 (SEQ ID NO: 45), C6orf167-A24-10-869 (SEQ ID NO: 46), C6orf167-A24-10-966 (SEQ ID NO: 47), C6orf167-A24-10-66 (SEQ ID NO: 48), C6orf167-A24-10-914 (SEQ ID NO: 49), C6orf167-A24-10-964 (SEQ ID NO: 50), C6orf167-A24-10-143 (SEQ ID NO: 51), C6orf167-A24-10-647 (SEQ ID NO: 52), C6orf167-A24-10-851 (SEQ ID NO: 53), C6orf167-A24-10-519 (SEQ ID NO: 54), C6orf167-A24-10-97 (SEQ ID NO: 55), C6orf167-A24-10-827 (SEQ ID NO: 56), C6orf167-A24-10-389 (SEQ ID NO: 57), C6orf167-A24-10-273 (SEQ ID NO: 58), C6orf167-A24-10-670 (SEQ ID NO: 59), C6orf167-A24-10-132 (SEQ ID NO: 60), and C6orf167-A24-10-1112 (SEQ ID NO: 61).

Moreover, after in vitro stimulation of T-cells by dendritic cells (DCs) loaded with these peptides, CTLs were successfully established using each of the following peptides:

C6orf167-A24-9-179 (SEQ ID NO: 2), C6orf167-A24-9-404 (SEQ ID NO: 4), C6orf167-A24-9-236 (SEQ ID NO: 7), C6orf167-A24-9-480 (SEQ ID NO: 8), C6orf167-A24-9-1170 (SEQ ID NO: 9), C6orf167-A24-9-9 (SEQ ID NO: 14), C6orf167-A24-9-530 (SEQ ID NO: 16), C6orf167-A24-9-315 (SEQ ID NO: 18), C6orf167-A24-9-132 (SEQ ID NO: 22), C6orf167-A24-9-851 (SEQ ID NO: 25), C6orf167-A24-9-55 (SEQ ID NO: 26), C6orf167-A24-9-220 (SEQ ID NO: 30), C6orf167-A24-10-626 (SEQ ID NO: 33), C6orf167-A24-10-429 (SEQ ID NO: 34), C6orf167-A24-10-917 (SEQ ID NO: 35), C6orf167-A24-10-474 (SEQ ID NO: 36), C6orf167-A24-10-254 (SEQ ID NO: 38), C6orf167-A24-10-194 (SEQ ID NO: 39), C6orf167-A24-10-956 (SEQ ID NO: 41), C6orf167-A24-10-511 (SEQ ID NO: 43), C6orf167-A24-10-315 (SEQ ID NO: 44), C6orf167-A24-10-598 (SEQ ID NO: 45), C6orf167-A24-10-966 (SEQ ID NO: 47), C6orf167-A24-10-66 (SEQ ID NO: 48), C6orf167-A24-10-914 (SEQ ID NO: 49), and C6orf167-A24-10-851 (SEQ ID NO: 53).

Candidates of HLA-A2 binding peptides derived from C6orf167 were identified based on their binding affinities to HLA-A2. The following peptides are considered to be candidate peptides for immunotherapy:

C6orf167-A2-9-202 (SEQ ID NO: 63), C6orf167-A2-9-227 (SEQ ID NO: 64), C6orf167-A2-9-855 (SEQ ID NO: 65), C6orf167-A2-9-131 (SEQ ID NO: 66), C6orf167-A2-9-533 (SEQ ID NO: 67), C6orf167-A2-9-858 (SEQ ID NO: 68), C6orf167-A2-9-290 (SEQ ID NO: 69), C6orf167-A2-9-647 (SEQ ID NO: 70), C6orf167-A2-9-929 (SEQ ID NO: 71), C6orf167-A2-9-219 (SEQ ID NO: 72), C6orf167-A2-9-904 (SEQ ID NO: 73), C6orf167-A2-9-648 (SEQ ID NO: 74), C6orf167-A2-9-133 (SEQ ID NO: 75), C6orf167-A2-9-887 (SEQ ID NO: 76), C6orf167-A2-9-319 (SEQ ID NO: 77), C6orf167-A2-9-667 (SEQ ID NO: 78), C6orf167-A2-9-261 (SEQ ID NO: 79), C6orf167-A2-9-965 (SEQ ID NO: 80), C6orf167-A2-9-964 (SEQ ID NO: 81), C6orf167-A2-9-578 (SEQ ID NO: 82), C6orf167-A2-9-623 (SEQ ID NO: 83), C6orf167-A2-9-484 (SEQ ID NO: 84), C6orf167-A2-9-457 (SEQ ID NO: 85), C6orf167-A2-9-253 (SEQ ID NO: 86), C6orf167-A2-9-671 (SEQ ID NO: 87), C6orf167-A2-9-283 (SEQ ID NO: 88), C6orf167-A2-9-1018 (SEQ ID NO: 89), C6orf167-A2-9-1091 (SEQ ID NO: 90), C6orf167-A2-9-1113 (SEQ ID NO: 91), C6orf167-A2-9-821 (SEQ ID NO: 92), C6orf167-A2-9-1116 (SEQ ID NO: 93), C6orf167-A2-9-528 (SEQ ID NO: 94), C6orf167-A2-9-1112 (SEQ ID NO: 95), C6orf167-A2-9-99 (SEQ ID NO: 96), C6orf167-A2-9-590 (SEQ ID NO: 97), C6orf167-A2-9-224 (SEQ ID NO: 98), C6orf167-A2-9-405 (SEQ ID NO: 99), C6orf167-A2-10-716(SEQ ID NO: 100), C6orf167-A2-10-535 (SEQ ID NO: 101), C6orf167-A2-10-226 (SEQ ID NO: 102), C6orf167-A2-10-303 (SEQ ID NO: 103), C6orf167-A2-10-311 (SEQ ID NO: 104), C6orf167-A2-10-425 (SEQ ID NO: 105), C6orf167-A2-10-554 (SEQ ID NO: 106), C6orf167-A2-10-648 (SEQ ID NO: 107), C6orf167-A2-10-569 (SEQ ID NO: 108), C6orf167-A2-10-202 (SEQ ID NO: 109), C6orf167-A2-10-527 (SEQ ID NO: 110), C6orf167-A2-10-10 (SEQ ID NO: 111), C6orf167-A2-10-577 (SEQ ID NO: 112), C6orf167-A2-10-128 (SEQ ID NO: 113), C6orf167-A2-10-622 (SEQ ID NO: 114), C6orf167-A2-10-178 (SEQ ID NO: 115), C6orf167-A2-10-47 (SEQ ID NO: 116), C6orf167-A2-10-219 (SEQ ID NO: 117), C6orf167-A2-10-1155 (SEQ ID NO: 118), C6orf167-A2-10-227 (SEQ ID NO: 119), C6orf167-A2-10-253 (SEQ ID NO: 120), C6orf167-A2-10-606 (SEQ ID NO: 121), C6orf167-A2-10-290 (SEQ ID NO: 122), C6orf167-A2-10-262 (SEQ ID NO: 123), C6orf167-A2-10-965 (SEQ ID NO: 124), C6orf167-A2-10-1113 (SEQ ID NO: 125), C6orf167-A2-10-77 (SEQ ID NO: 126), C6orf167-A2-10-319 (SEQ ID NO: 127), C6orf167-A2-10-1022 (SEQ ID NO: 128), C6orf167-A2-10-910 (SEQ ID NO: 129), C6orf167-A2-10-738 (SEQ ID NO: 130), C6orf167-A2-10-482 (SEQ ID NO: 131), C6orf167-A2-10-282 (SEQ ID NO: 132), C6orf167-A2-10-442 (SEQ ID NO: 133), C6orf167-A2-10-625 (SEQ ID NO: 134), C6orf167-A2-10-1120 (SEQ ID NO: 135), C6orf167-A2-10-640 (SEQ ID NO: 136), C6orf167-A2-10-619 (SEQ ID NO: 137), C6orf167-A2-10-747 (SEQ ID NO: 138), C6orf167-A2-10-1131 (SEQ ID NO: 139), C6orf167-A2-10-71 (SEQ ID NO: 140), C6orf167-A2-10-614 (SEQ ID NO: 141), C6orf167-A2-10-457 (SEQ ID NO: 142), C6orf167-A2-10-1001 (SEQ ID NO: 143), C6orf167-A2-10-397 (SEQ ID NO: 144), C6orf167-A2-10-268 (SEQ ID NO: 145), C6orf167-A2-10-1088 (SEQ ID NO: 146), C6orf167-A2-10-528 (SEQ ID NO: 147), C6orf167-A2-10-1049 (SEQ ID NO: 148), C6orf167-A2-10-886 (SEQ ID NO: 149), C6orf167-A2-10-411 (SEQ ID NO: 150) and C6orf167-A2-10-579 (SEQ ID NO: 151).

Moreover, after in vitro stimulation of T-cells by dendritic cells (DCs) pulsed (loaded) with these peptides, CTLs were successfully established using each of the following peptides;

C6orf167-A2-9-855 (SEQ ID NO: 65), C6orf167-A2-9-131 (SEQ ID NO: 66), C6orf167-A2-9-887 (SEQ ID NO: 76), C6orf167-A2-9-261 (SEQ ID NO: 79), C6orf167-A2-9-484 (SEQ ID NO: 84), C6orf167-A2-10-535 (SEQ ID NO: 101), C6orf167-A2-10-527 (SEQ ID NO: 110), C6orf167-A2-10-10 (SEQ ID NO: 111), C6orf167-A2-10-577 (SEQ ID NO: 112), C6orf167-A2-10-128 (SEQ ID NO: 113), C6orf167-A2-10-622 (SEQ ID NO: 114), C6orf167-A2-10-219 (SEQ ID NO: 117), C6orf167-A2-10-1155 (SEQ ID NO: 118), C6orf167-A2-10-606 (SEQ ID NO: 121), C6orf167-A2-10-290 (SEQ ID NO: 122), C6orf167-A2-10-262 (SEQ ID NO: 123) and C6orf167-A2-10-965 (SEQ ID NO: 124).

These established CTLs show potent specific CTL activity against target cells pulsed with respective peptides. These results herein demonstrate that C6orf167 is an antigen recognized by CTL and that the peptides are epitope peptides of C6orf167 restricted by HLA-A24 or HLA-A2.

Since the C6orf167 gene is over-expressed in cancer cells and tissues, including for example those of bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor, and not expressed in most normal organs, it represents a good target for immunotherapy. Thus, the present invention provides nonapeptides (peptides consisting of nine amino acid residues) and decapeptides (peptides consisting of ten amino acid residues) corresponding to CTL-recognized epitopes from C6orf167. Particularly preferred examples of nonapeptides and decapeptides of the present invention include those peptides having an amino acid sequence selected from among SEQ ID NOs: 1 to 61 and 63 to 151.

Generally, software programs now available, for example, on the Internet, such as those described in Parker K C et al., J Immunol 1994 Jan. 1, 152(1): 163-75 and Nielsen M et al., Protein Sci 2003; 12: 1007-17 can be used to calculate the binding affinities between various peptides and HLA antigens in silico. Binding affinity with HLA antigens can be measured as described, for example, in Parker K C et al., J Immunol 1994 Jan. 1, 152(1): 163-75, Kuzushima K et al., Blood 2001, 98(6): 1872-81, Larsen M V et al. BMC Bioinformatics. 2007 Oct. 31; 8: 424, Buus S et al. Tissue Antigens., 62:378-84, 2003, Nielsen M et al., Protein Sci 2003; 12: 1007-17, and Nielsen M et al. PLoS ONE 2007; 2: e796, which are summarized in, e.g., Lafuente E M et al., Current Pharmaceutical Design, 2009, 15, 3209-3220. The methods for determining binding affinity is described, for example, in; Journal of Immunological Methods, 1995, 185: 181-190; Protein Science, 2000, 9: 1838-1846. Therefore, one can select fragments derived from C6orf167, which have high binding affinity with HLA antigens using such software programs. Thus, the present invention encompasses peptides composed of any fragments derived from C6orf167, which would be determined to bind with HLA antigens by such known programs. Furthermore, such peptides may include the peptide consisting of the full length of C6orf167.

The nonapeptides and decapeptides of the present invention can be flanked with additional amino acid residues, so long as the resulting peptide retains its CTL inducibility. The additional amino acid residues can be composed of any kind of amino acids, so long as they do not impair the CTL inducibility of the original peptide. Thus, the present invention encompasses peptides with binding affinity to HLA antigens, including peptides derived from C6orf167. Such peptides are, for example, less than about 40 amino acids, often less than about 20 amino acids, and usually less than about 15 amino acids.

In general, the modification of one, two or more amino acids in a peptide will not influence the function of the peptide, and in some cases will even enhance the desired function of the original protein. In fact, modified peptides (i.e., peptides composed of an amino acid sequence, in which one, two or several amino acid residues have been modified (i.e., substituted, added or inserted) as compared to an original reference sequence) have been known to retain the biological activity of the original peptide (Mark et al., Proc Natl Acad Sci USA 1984, 81: 5662-6; Zoller and Smith, Nucleic Acids Res 1982, 10: 6487-500; Dalbadie-McFarland et al., Proc Natl Acad Sci USA 1982, 79: 6409-13). Thus, in one embodiment, the peptides of the present invention have both CTL inducibility and an amino acid sequence selected from among SEQ ID NOs: 1 to 61 and 63 to 151, wherein one, two or even more amino acids are added and/or substituted.

Those of skill in the art will recognize that individual additions or substitutions to an amino acid sequence that alter a single amino acid or a small percentage of amino acids tend to result in the conservation of the properties of the original amino acid side-chain. As such, they are often referred to as “conservative substitutions” or “conservative modifications”, wherein the alteration of a protein results in a modified protein having a function analogous to the original protein. Conservative substitution tables providing functionally similar amino acids are well known in the art. Examples of amino acid side-chains characteristics that are desirable to conserve include, for example: hydrophobic amino acids (A, I, L, M, F, P, W, Y, V), hydrophilic amino acids (R, D, N, C, E, Q, G, H, K, S, T), and side-chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). In addition, the following eight groups each contain amino acids that are accepted in the art as conservative substitutions for one another:

### 1) Alanine (A), Glycine (G);

2) Aspartic acid (D), Glutamic acid (E);

### 3) Aspargine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

8) Cysteine (C), Methionine (M) (see, e.g., Creighton, Proteins 1984).

Such conservatively modified peptides are also considered to be peptides of the present invention. However, peptides of the present invention are not restricted thereto and can include non-conservative modifications, so long as the modified peptide retains the CTL inducibility of the original peptide. Furthermore, modified peptides should not exclude CTL inducible peptides of polymorphic variants, interspecies homologues, and alleles of C6orf167.

To retain the requisite CTL inducibility, one can modify (insert, add and/or substitute) a small number (for example, 1, 2 or several) or a small percentage of amino acids. Herein, the term “several” means 5 or fewer amino acids, for example, 4 or 3 or fewer. The percentage of amino acids to be modified is preferably 20% or less, more preferably 15% of less, and even more preferably 10% or less or 1 to 5%.

When used in the context of immunotherapy, the peptides of the present invention should be presented on the surface of a cell or exosome, preferably as a complex with an HLA antigen. Therefore, it is preferable to select peptides that not only induce CTLs but also possess high binding affinity to the HLA antigen. To that end, the peptides can be modified by substitution, insertion, and/or addition of the amino acid residues to yield a modified peptide having improved binding affinity. In addition to peptides that are naturally displayed, since the regularity of the sequences of peptides displayed by binding to HLA antigens is already known (J Immunol 1994, 152: 3913; Immunogenetics 1995, 41: 178; J Immunol 1994, 155: 4307), modifications based on such regularity can be introduced into the immunogenic peptides of the invention.

For example, it may be desirable to substitute the second amino acid from the N-terminus with leucine or methionine, and/or the amino acid at the C-terminus with valine or leucine in order to increase the HLA-A24 binding affinity. Thus, peptides having an amino acid sequence selected from among SEQ ID NOs: 1 to 61, wherein the second amino acid from the N-terminus of the amino acid sequence of the SEQ ID NO is substituted with leucine or methionine, and/or wherein the C-terminus of the amino acid sequence of the SEQ ID NO is substituted with valine or leucine are encompassed by the present invention.

Alternatively, it may be desirable to substitute the second amino acid from the N-terminus with phenylalanine, tyrosine, methionine, or tryptophan, and/or the amino acid at the C-terminus with phenylalanine, leucine, isoleucine, tryptophan, or methionine in order to increase the HLA-A2 binding affinity. Thus, peptides having an amino acid sequence selected from among SEQ ID NOs: 63 to 151, wherein the second amino acid from the N-terminus of the amino acid sequence of the SEQ ID NO is substituted with phenylalanine, tyrosine, methionine, or tryptophan, and/or wherein the C-terminus of the amino acid sequence of the SEQ ID NO is substituted with phenylalanine, leucine, isoleucine, tryptophan, or methionine are encompassed by the present invention.

Substitutions can be introduced not only at the terminal amino acids but also at the position of potential T cell receptor (TCR) recognition of peptides. Several studies have demonstrated that a peptide with amino acid substitutions can be equal to or better than the original, for example CAP1, p53₍₂₆₄₋₂₇₂₎, Her-2/neu₍₃₆₉₋₃₇₇₎ or gp100₍₂₀₉₋₂₁₇₎ (Zaremba et al. Cancer Res. 57, 4570-4577, 1997, T. K. Hoffmann et al. J. Immunol. (2002) Feb. 1; 168(3):1338-47, S. O. Dionne et al. Cancer Immunol immunother. (2003) 52: 199-206 and S. O. Dionne et al. Cancer Immunology, Immunotherapy (2004) 53, 307-314).

The present invention also contemplates the addition of one, two or several amino acids can also be added to the N and/or C-terminus of the present peptides. Such modified peptides having high HLA antigen binding affinity and retained CTL inducibility are also included in the present invention.

However, when the peptide sequence is identical to a portion of the amino acid sequence of an endogenous or exogenous protein having a different function, side effects such as autoimmune disorders and/or allergic symptoms against specific substances may be induced. Therefore, it is preferable to first perform homology searches using available databases to avoid situations in which the sequence of the peptide matches the amino acid sequence of another protein. When it becomes clear from the homology searches that there exists not even a peptide with 1 or 2 amino acid differences as compared to the objective peptide, the objective peptide can be modified in order to increase its binding affinity with HLA antigens, and/or increase its CTL inducibility without any danger of such side effects.

Although peptides having high binding affinity to the HLA antigens as described above are expected to be highly effective, the candidate peptides, which are selected according to the presence of high binding affinity as an indicator, are further examined for the presence of CTL inducibility. Herein, the phrase “CTL inducibility” indicates the ability of the peptide to induce cytotoxic T lymphocytes (CTLs) when presented on antigen-presenting cells (APCs). Further, “CTL inducibility” includes the ability of the peptide to induce CTL activation, CTL proliferation, promote CTL lysis of target cells, and to increase CTL IFN-gamma production.

Confirmation of CTL inducibility is accomplished by inducing APCs carrying human MHC antigens (for example, B-lymphocytes, macrophages, and dendritic cells (DCs)), or more specifically DCs derived from human peripheral blood mononuclear leukocytes, and after stimulation with the peptides, mixing with CD8 positive cells, and then measuring the IFN-gamma produced and released by CTL against the target cells. As the reaction system, transgenic animals that have been produced to express a human HLA antigen (for example, those described in BenMohamed L, Krishnan R, Longmate J, Auge C, Low L, Primus J, Diamond DJ, Hum Immunol 2000 August, 61(8): 764-79, Related Articles, Books, Linkout Induction of CTL response by a minimal epitope vaccine in HLA A*0201/DR1 transgenic mice: dependence on HLA class II restricted T(H) response) can be used. For example, the target cells can be radiolabeled with ⁵¹Cr and such, and cytotoxic activity can be calculated from radioactivity released from the target cells. Alternatively, CTL inducibility can be assessed by measuring IFN-gamma produced and released by CTL in the presence of APCs that carry immobilized peptides, and visualizing the inhibition zone on the media using anti-IFN-gamma monoclonal antibodies.

As a result of examining the CTL inducibility of the peptides as described above, it was discovered that nonapeptides or decapeptides selected from among the amino acid sequences indicated by SEQ ID NOs: 2, 4, 7, 8, 9, 14, 16, 18, 22, 25, 26, 30, 33, 34, 35, 36, 38, 39, 41, 43, 44, 45, 47, 48, 49, 53, 65, 66, 76, 79, 84, 101, 110, 111, 112, 113, 114, 117, 118, 121, 122, 123, and 124 showed particularly high CTL inducibility as well as high binding affinity to an HLA antigen. Thus, these peptides are exemplified preferred embodiments of the present invention.

Furthermore, the result of homology analysis showed that those peptides do not have significant homology with peptides derived from any other known human gene products. Accordingly, the possibility of unknown or undesired immune responses arising when used for immunotherapy is lowered. Therefore, also from this aspect, these peptides find use for eliciting immunity against C6orf167 in cancer patients. Thus, the peptides of the present invention, preferably, peptides having an amino acid sequence selected from among SEQ ID NOs: 2, 4, 7, 8, 9, 14, 16, 18, 22, 25, 26, 30, 33, 34, 35, 36, 38, 39, 41, 43, 44, 45, 47, 48, 49, 53, 65, 66, 76, 79, 84, 101, 110, 111, 112, 113, 114, 117, 118, 121, 122, 123, and 124.

In addition to the above-described modifications, the peptides of the present invention can also be linked to other peptides, so long as the resulting linked peptide retains the requisite CTL inducibility of the original peptide. Examples of suitable peptides include: the peptides of the present invention or the CTL-inducible peptides derived from other TAAs. Suitable inter-peptide linkers are well known in the art and include, for example AAY (P. M. Daftarian et al., J Trans Med 2007, 5:26), AAA, NKRK (R. P. M. Sutmuller et al., J. Immunol. 2000, 165: 7308-7315) or K (S. Ota et al., Can Res. 62, 1471-1476, K. S. Kawamura et al., J. Immunol. 2002, 168: 5709-5715).

For example, non-C6orf167 tumor associated antigen peptides also can be used substantially simultaneously to increase the immune response via HLA class I and/or class II. It is well established that cancer cells can express more than one tumor associated gene. Thus, it is within the scope of routine experimentation for one of ordinary skill in the art to determine whether a particular subject expresses additional tumor associated genes, and then to include HLA class I and/or HLA class II binding peptides derived from expression products of such genes in C6orf167 compositions or vaccines according to the present invention.

Examples of HLA class I and HLA class II binding peptides are known to those of ordinary skill in the art (for example, see Coulie, Stem Cells 13:393-403, 1995), and can be used in the invention in a like manner as those disclosed herein. Thus, one of ordinary skill in the art can readily prepare polypeptides including one or more C6orf167 peptides and one or more of the non-C6orf167 peptides, or nucleic acids encoding such polypeptides, using standard procedures of molecular biology.

The above such linked peptides are referred to herein as “polytopes”, i.e., groups of two or more potentially immunogenic or immune response stimulating peptides which can be joined together in various arrangements (e.g., concatenated, overlapping). The polytope (or nucleic acid encoding the polytope) can be administered in a standard immunization protocol, e.g., to animals, to test the effectiveness of the polytope in stimulating, enhancing and/or provoking an immune response.

The peptides can be joined together directly or via the use of flanking sequences to form polytopes, and the use of polytopes as vaccines is well known in the art (see, e.g., Thomson et al., Proc. Natl. Acad. Sci. USA 92(13):5845-5849, 1995; Gilbert et al., Nature Biotechnol. 15(12):1280-1284, 1997; Thomson et al., J. Immunol. 157(2):822-826, 1996; Tarn et al., J. Exp. Med. 171(1):299-306, 1990). Polytopes containing various numbers and combinations of epitopes can be prepared and tested for recognition by CTLs and for efficacy in increasing an immune response.

The peptides of the present invention can also be linked to other substances, so long as the resulting linked peptide retains the requisite CTL inducibility of the original peptide. Examples of suitable substances include, for example: peptides, lipids, sugar and sugar chains, acetyl groups, natural and synthetic polymers, etc. The peptides can contain modifications such as glycosylation, side chain oxidation, or phosphorylation, etc., provided the modifications do not destroy the biological activity of the original peptide. These kinds of modifications can be performed to confer additional functions (e.g., targeting function, and delivery function) or to stabilize the peptide.

For example, to increase the in vivo stability of a peptide, it is known in the art to introduce D-amino acids, amino acid mimetics or unnatural amino acids; this concept can also be adapted to the present peptides. The stability of a peptide can be assayed in a number of ways. For instance, peptidases and various biological media, such as human plasma and serum, can be used to test stability (see, e.g., Verhoef et al., Eur J Drug Metab Pharmacokin 1986, 11: 291-302).

Moreover, as noted above, among the modified peptides that are substituted, deleted or added by one, two or several amino acid residues, those having same or higher activity as compared to original peptides can be screened for or selected. The present invention, therefore, also provides the method of screening for or selecting modified peptides having same or higher activity as compared to originals. An illustrative method includes the steps of:

a: substituting, deleting or adding at least one amino acid residue of a peptide of the present invention,

b: determining the activity of the peptide,

c: selecting the peptide having same or higher activity as compared to the original.

Herein, the activity to be assayed may include MHC binding activity, APC or CTL inducibility and cytotoxic activity.

Herein, the peptides of the present invention can also be described as “C6orf167 peptide(s)” or “C6orf167 polypeptide(s)”.

### III. Preparation of C6orf167 Peptides

The peptides of the present invention can be prepared using well known techniques. For example, the peptides can be prepared synthetically, using recombinant DNA technology or chemical synthesis. The peptides of the present invention can be synthesized individually or as longer polypeptides including two or more peptides. The peptides can then be isolated i.e., purified or isolated so as to be substantially free of other naturally occurring host cell proteins and fragments thereof, or any other chemical substances.

The peptides of the present invention may contain modifications, such as glycosylation, side chain oxidation, or phosphorylation, provided the modifications do not destroy the biological activity of the original peptide. Other illustrative modifications include incorporation of D-amino acids or other amino acid mimetics that can be used, for example, to increase the serum half life of the peptides.

Peptides of the present invention can be obtained through chemical synthesis based on the selected amino acid sequence. Examples of conventional peptide synthesis methods that can be adapted for the synthesis include:

(i) Peptide Synthesis, Interscience, New York, 1966;

(ii) The Proteins, Vol. 2, Academic Press, New York, 1976;

(iii) Peptide Synthesis (in Japanese), Maruzen Co., 1975;

(iv) Basics and Experiment of Peptide Synthesis (in Japanese), Maruzen Co., 1985;

(v) Development of Pharmaceuticals (second volume) (in Japanese), Vol. 14 (peptide synthesis), Hirokawa, 1991;

(vi) WO99/67288; and

(vii) Barany G. & Merrifield R. B., Peptides Vol. 2, “Solid Phase Peptide Synthesis”, Academic Press, New York, 1980, 100-118.

Alternatively, the present peptides can be obtained adapting any known genetic engineering method for producing peptides (e.g., Morrison J, J Bacteriology 1977, 132: 349-51; Clark-Curtiss & Curtiss, Methods in Enzymology (eds. Wu et al.) 1983, 101: 347-62). For example, first, a suitable vector harboring a polynucleotide encoding the objective peptide in an expressible form (e.g., downstream of a regulatory sequence corresponding to a promoter sequence) is prepared and transformed into a suitable host cell. The host cell is then cultured to produce the peptide of interest. The peptide can also be produced in vitro adopting an in vitro translation system.

### IV. Polynucleotides

The present invention also provides a polynucleotide which encodes any of the aforementioned peptides of the present invention. These include polynucleotides derived from the natural occurring C6orf167 gene (GenBank Accession No. NM_{—}198468.2 (SEQ ID NO: 158)) as well as those having a conservatively modified nucleotide sequence thereof. Herein, the phrase “conservatively modified nucleotide sequence” refers to sequences which encode identical or essentially identical amino acid sequences. Due to the degeneracy of the genetic code, a large number of functionally identical nucleic acids encode any given protein. For instance, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a peptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a peptide is implicitly described in each disclosed sequence.

The polynucleotide of the present invention can be composed of DNA, RNA, and derivatives thereof. A DNA is suitably composed of bases such as A, T, C, and G, and T is replaced by U in an RNA.

The polynucleotide of the present invention can encode multiple peptides of the present invention with or without intervening amino acid sequences in between. For example, the intervening amino acid sequence can provide a cleavage site (e.g., enzyme recognition sequence) of the polynucleotide or the translated peptides. Furthermore, the polynucleotide can include any additional sequences to the coding sequence encoding the peptide of the present invention. For example, the polynucleotide can be a recombinant polynucleotide that includes regulatory sequences required for the expression of the peptide or can be an expression vector (plasmid) with marker genes and such. In general, such recombinant polynucleotides can be prepared by the manipulation of polynucleotides through conventional recombinant techniques using, for example, polymerases and endonucleases.

Both recombinant and chemical synthesis techniques can be used to produce the polynucleotides of the present invention. For example, a polynucleotide can be produced by insertion into an appropriate vector, which can be expressed when transfected into a competent cell. Alternatively, a polynucleotide can be amplified using PCR techniques or expression in suitable hosts (see, e.g., Sambrook et al., Molecular Cloning: A Laboratory Manual, Cold Spring Harbor Laboratory, New York, 1989). Alternatively, a polynucleotide can be synthesized using the solid phase techniques, as described in Beaucage SL & Iyer R P, Tetrahedron 1992, 48: 2223-311; Matthes et al., EMBO J. 1984, 3: 801-5.

### V. Exosomes

The present invention further provides intracellular vesicles called exosomes, which present complexes formed between the peptides of the present invention and HLA antigens on their surface. Exosomes can be prepared, for example using the methods detailed in Japanese Patent Application Kohyo Publications Nos. Hei 11-510507 and WO99/03499, and can be prepared using APCs obtained from patients who are subject to treatment and/or prevention. The exosomes of the present invention can be inoculated as vaccines, in a fashion similar to the peptides of the present invention.

The type of HLA antigens included in the complexes must match that of the subject requiring treatment and/or prevention. For example, in the Japanese population, HLA-A24 and HLA-A2, particularly HLA-A*2402 and HLA-A*0201 and HLA-A*0206, are prevalent and therefore would be appropriate for treatment of Japanese patients. The use of the A24 type that are highly expressed among the Japanese and Caucasian is favorable for obtaining effective results, and subtypes such as A2402, A*0201 and A*0206 also find use. Typically, in the clinic, the type of HLA antigen of the patient requiring treatment is investigated in advance, which enables the appropriate selection of peptides having high levels of binding affinity to the particular antigen, or having CTL inducibility by antigen presentation. Furthermore, in order to obtain peptides having both high binding affinity and CTL inducibility, substitution, insertion and/or addition of 1, 2, or several amino acids can be performed based on the amino acid sequence of the naturally occurring C6orf167 partial peptide.

When using the A24 type HLA antigen for the exosome of the present invention, peptides having a sequence selected from among SEQ ID NOs: 1 to 61 find use.

Alternatively, when using the A2 type HLA antigen for the exosome of the present invention, peptides having a sequence selected from among SEQ ID NOs: 63 to 151 find use.

### VI. Antigen-Presenting Cells (APCs)

The present invention also provides isolated antigen-presenting cells (APCs) that present complexes formed between HLA antigens and the peptides of the present invention on its surface. The APCs can be derived from patients who are subject to treatment and/or prevention, and can be administered as vaccines by themselves or in combination with other drugs including the peptides of the present invention, exosomes, or CTLs.

The APCs are not limited to a particular kind of cells and include dendritic cells (DCs), Langerhans cells, macrophages, B cells, and activated T cells, which are known to present proteinaceous antigens on their cell surface so as to be recognized by lymphocytes. Since DC is a representative APC having the strongest CTL inducing action among APCs, DCs find use as the APCs of the present invention.

For example, the APCs of the present invention can be obtained by inducing DCs from peripheral blood monocytes and then contacting (stimulating) them with the peptides of the present invention in vitro, ex vivo or in vivo. When the peptides of the present invention are administered to the subjects, APCs that present the peptides of the present invention are induced in the body of the subject. The phrase “inducing APC” includes contacting (stimulating) a cell with the peptides of the present invention, or nucleotides encoding the peptides of the present invention to present complexes formed between HLA antigens and the peptides of the present invention on cell's surface. Therefore, the APCs of the present invention can be obtained by collecting the APCs from the subject after administering the peptides of the present invention to the subject. Alternatively, the APCs of the present invention can be obtained by contacting APCs collected from a subject with the peptide of the present invention.

The APCs of the present invention can be administered to a subject for inducing immune response against cancer in the subject by themselves or in combination with other drugs including the peptides, exosomes or CTLs of the present invention. For example, the ex vivo administration can include steps of:

a: collecting APCs from a first subject,

b: contacting with the APCs of step a, with the peptide, and

c: administering the APCs of step b to a second subject.

The first subject and the second subject can be the same individual, or may be different individuals. Alternatively, according to the present invention, use of the peptides of the present invention for manufacturing a pharmaceutical composition inducing antigen-presenting cells is provided. In addition, the present invention provides a method or process for manufacturing a pharmaceutical composition inducing antigen-presenting cells. Further, the present invention also provides the peptides of the present invention for inducing antigen-presenting cells. The APCs obtained by step b can be a vaccine for treating and/or preventing cancer, such as bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor, but not limited thereto.

According to an aspect of the present invention, the APCs have a high level of CTL inducibility. In the term of “high level of CTL inducibility”, the high level is relative to the level of that by APC contacted with no peptide or peptides which can not induce the CTL. Such APCs having a high level of CTL inducibility can be prepared by a method that includes the step of transferring a polynucleotide encoding the peptide of the present invention to APCs in vitro as well as the method mentioned above. The introduced genes can be in the form of DNAs or RNAs. Examples of methods for introduction include, without particular limitations, various methods conventionally performed in this field, such as lipofection, electroporation, and calcium phosphate method can be used. More specifically, it can be performed as described in Cancer Res 1996, 56: 5672-7; J Immunol 1998, 161: 5607-13; J Exp Med 1996, 184: 465-72; Published Japanese Translation of International Publication No. 2000-509281. By transferring the gene into APCs, the gene undergoes transcription, translation, and such in the cell, and then the obtained protein is processed by MHC Class I or Class II, and proceeds through a presentation pathway to present partial peptides.

### VII. Cytotoxic T Lymphocytes (CTLs)

A CTL induced against any one of the peptides of the present invention strengthens the immune response targeting cancer cells in vivo and thus can be used as vaccines, in a fashion similar to the peptides per se. Thus, the present invention provides isolated CTLs that are specifically induced or activated by any one of the present peptides.

Such CTLs can be obtained by (1) administering the peptide(s) of the present invention to a subject or (2) contacting (stimulating) subject-derived APCs, and CD8 positive cells, or peripheral blood mononuclear leukocytes in vitro with the peptide(s) of the present invention or (3) contacting CD8 positive cells or peripheral blood mononuclear leukocytes in vitro with the APCs or exosomes presenting a complex of an HLA antigen and the peptide on its surface or (4) introducing a gene that includes a polynucleotide encoding a T cell receptor (TCR) subunit biding to the peptide of the present invention. Such APCs or exosomes can be prepared by the methods described above and details of the method of (4) is described bellow in section “VIII. T cell receptor (TCR)”.

The CTLs of the present invention can be derived from patients who are subject to treatment and/or prevention, and can be administered by themselves or in combination with other drugs including the peptides of the present invention or exosomes for the purpose of regulating effects. The obtained CTLs act specifically against target cells presenting the peptides of the present invention, for example, the same peptides used for induction. The target cells can be cells that endogenously express C6orf167, such as cancer cells, or cells that are transfected with the C6orf167 gene; and cells that present a peptide of the present invention on the cell surface due to stimulation by the peptide can also serve as targets of activated CTL attack.

### VIII. T Cell Receptor (TCR)

The present invention also provides a composition including nucleic acids encoding polypeptides that are capable of forming a subunit of a T cell receptor (TCR), and methods of using the same. The TCR subunits have the ability to form TCRs that confer specificity to T cells against tumor cells presenting C6orf167. By using the known methods in the art, the nucleic acids of alpha- and beta-chains as the TCR subunits of the CTL induced with one or more peptides of the present invention can be identified (WO2007/032255 and Morgan et al., J Immunol, 171, 3288 (2003)). For example, the PCR method is preferred to analyze the TCR. The PCR primers for the analysis can be, for example, 5′-R primers (5′-gtctaccaggcattcgcttcat-3′) as 5′ side primers (SEQ ID NO: 160) and 3-TRa-C primers (5′-tcagctggaccacagccgcagcgt-3′) specific to TCR alpha chain C region (SEQ ID NO: 161), 3-TRb-C1 primers (5′-tcagaaatcctttctcttgac-3′) specific to TCR beta chain C1 region (SEQ ID NO: 162) or 3-TRbeta-C2 primers (5′-ctagcctctggaatcctttctctt-3′) specific to TCR beta chain C2 region (SEQ ID NO: 163) as 3′ side primers, but not limited thereto. The derivative TCRs can bind target cells displaying the C6orf167 peptide with high avidity, and optionally mediate efficient killing of target cells presenting the C6orf167 peptide in vivo and in vitro.

The nucleic acids encoding the TCR subunits can be incorporated into suitable vectors, e.g., retroviral vectors. These vectors are well known in the art. The nucleic acids or the vectors including them usefully can be transferred into a T cell, for example, a T cell from a patient. Advantageously, the invention provides an off-the-shelf composition allowing rapid modification of a patient's own T cells (or those of another mammal) to rapidly and easily produce modified T cells having excellent cancer cell killing properties.

The nucleic acids encoding the TCR subunits may be incorporated into suitable vectors, e.g., retroviral vectors. These vectors are well known in the art. The nucleic acids or the vectors including them usefully may be transferred into a T cell, for example, a T cell from a patient. Advantageously, the present invention provides an off-the-shelf composition allowing rapid modification of a patient's own T cells (or those of another mammal) to rapidly and easily produce modified T cells having excellent cancer cell killing properties.

The specific TCR is a receptor capable of specifically recognizing a complex of a peptide of the present invention and HLA molecule, giving a T cell specific activity against the target cell when the TCR is presented on the surface of the T cell. A specific recognition of the above complex may be confirmed by any known methods, preferred examples of which include HLA multimer staining analysis using HLA molecules and peptides of the present invention, and ELISPOT assay. By performing the ELISPOT assay, it can be confirmed that a T cell expressing the TCR on the cell surface recognizes a cell by the TCR, and that the signal is transmitted intracellularly. The confirmation that the above-mentioned complex can give a T cell cytotoxic activity when the complex exists on the T cell surface may also be carried out by a known method. A preferred method includes, for example, the determination of cytotoxic activity against an HLA positive target cell, such as chromium release assay.

Also, the present invention provides CTLs which are prepared by transduction with the nucleic acids encoding the TCR subunits polypeptides that bind to the C6orf167 peptide, e.g., SEQ ID NOs: 1 to 61 in the context of HLA-A24, and also the peptides of SEQ ID NOs: 63 to 151 in the context of HLA-A2.

The transduced CTLs are capable of homing to cancer cells in vivo, and can be expanded by well known culturing methods in vitro (e.g., Kawakami et al., J. Immunol., 142, 3452-3461 (1989)). The CTLs of the present invention can be used to form an immunogenic composition useful in treating or the prevention of cancer in a patient in need of therapy or protection (See WO2006/031221 the contents of which are incorporated by reference herein).

Prevention and prophylaxis include any activity which reduces the burden of mortality or morbidity from disease. Prevention and prophylaxis can occur “at primary, secondary and tertiary prevention levels.” While primary prevention and prophylaxis avoid the development of a disease, secondary and tertiary levels of prevention and prophylaxis encompass activities aimed at the prevention and prophylaxis of the progression of a disease and the emergence of symptoms as well as reducing the negative impact of an already established disease by restoring function and reducing disease-related complications. Alternatively, prevention and prophylaxis include a wide range of prophylactic therapies aimed at alleviating the severity of the particular disorder, e.g., reducing the proliferation and metastasis of tumors, reducing angiogenesis.

Treating and/or for the prophylaxis of cancer or, and/or the prevention of post-operative recurrence thereof includes any of the following steps, such as surgical removal of cancer cells, inhibition of the growth of cancerous cells, involution or regression of a tumor, induction of remission and suppression of occurrence of cancer, tumor regression, and reduction or inhibition of metastasis. Effectively treating and/or the prophylaxis of cancer decreases mortality and improves the prognosis of individuals having cancer, decreases the levels of tumor markers in the blood, and alleviates detectable symptoms accompanying cancer. For example, reduction or improvement of symptoms constitutes effectively treating and/or the prophylaxis include 10%, 20%, 30% or more reduction, or stable disease.

### IX. Pharmaceutical Compositions

Since C6orf167 expression is specifically elevated in cancers, examples of which include, but are not limited to, bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor, as compared with normal tissue, the peptides of the present invention or polynucleotides encoding such peptides can be used for the treatment and/or prophylaxis of cancer, and/or for the prevention of postoperative recurrence thereof. Thus, the present invention provides a pharmaceutical composition for treating and/or preventing cancer, and/or preventing the postoperative recurrence thereof, such composition including one or more of the peptides, or polynucleotides of the present invention as an active ingredient. Alternatively, the present peptides can be expressed on the surface of any of the foregoing exosomes or cells, such as APCs for the use as pharmaceutical compositions. In addition, the aforementioned CTLs which target any one of the peptides of the present invention can also be used as the active ingredient of the present pharmaceutical substances and compositions.

The pharmaceutical substances and compositions of the present invention also find use as a vaccine. In the context of the present invention, the phrase “vaccine” (also referred to as an “immunogenic composition”) refers to a substance that has the function to induce anti-tumor immunity upon inoculation into animals.

The pharmaceutical compositions of the present invention can be used to treat and/or prevent cancers, and/or prevention of postoperative recurrence thereof in subjects or patients including human and any other mammal including, but not limited to, mouse, rat, guinea-pig, rabbit, cat, dog, sheep, goat, pig, cattle, horse, monkey, baboon, and chimpanzee, particularly a commercially important animal or a domesticated animal.

In another embodiment, the present invention also provides the use of an active ingredient selected from among:

(a) a peptide of the present invention;

(b) a nucleic acid encoding such a peptide as disclosed herein in an expressible form;

(c) an APC or an exosome presenting a peptide of the present invention on its surface; and

(d) a cytotoxic T cell of the present invention

in manufacturing a pharmaceutical composition or substance for treating or preventing cancer or tumor.

Alternatively, the present invention further provides an active ingredient selected from among:

(a) a peptide of the present invention;

(b) a nucleic acid encoding such a peptide as disclosed herein in an expressible form;

(c) an APC or an exosome presenting a peptide of the present invention on its surface; and

(d) a cytotoxic T cell of the present invention

for use in treating or preventing cancer of tumor.

Alternatively, the present invention further provides a method or process for manufacturing a pharmaceutical composition or substance for treating or preventing cancer or tumor, wherein the method or process includes the step of formulating a pharmaceutically or physiologically acceptable carrier with an active ingredient selected from among:

(a) a peptide of the present invention;

(b) a nucleic acid encoding such a peptide as disclosed herein in an expressible form;

(c) an APC or an exosome presenting a peptide of the present invention on its surface;

and

(d) a cytotoxic T cell of the present invention

as active ingredients.

In another embodiment, the present invention also provides a method or process for manufacturing a pharmaceutical composition or substance for treating or preventing cancer or tumor, wherein the method or process includes the steps of admixing an active ingredient with a pharmaceutically or physiologically acceptable carrier, wherein the active ingredient is selected from among:

(a) a peptide of the present invention;

(b) a nucleic acid encoding such a peptide as disclosed herein in an expressible form;

(c) an APC or an exosome presenting a peptide of the present invention on its surface; and

(d) a cytotoxic T cell of the present invention.

According to the present invention, peptides having an amino acid sequence selected from among SEQ ID NOs: 1 to 61 have been found to be HLA-A24 restricted epitope peptides or the candidates and also SEQ ID NOs: 63 to 151 have been found of be HLA-A2 restricted epitope peptides or the candidates that can induce potent and specific immune response. Therefore, the present pharmaceutical compositions which include any of these peptides with the amino acid sequences of SEQ ID NOs: 1 to 61 and 63 to 151 are particularly suited for the administration to subjects whose HLA antigen is HLA-A24 and HLA-A2 respectively. The same applies to pharmaceutical compositions that contain polynucleotides encoding any of these peptides (i.e., the polynucleotides of the present invention).

Cancers to be treated by the pharmaceutical compositions of the present invention are not limited and include all kinds of cancers wherein C6orf167 is involved, including, for example, bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor.

The present pharmaceutical compositions can contain in addition to the aforementioned active ingredients, other peptides which have the ability to induce CTLs against cancerous cells, other polynucleotides encoding the other peptides, other cells that present the other peptides, or such. Herein, the other peptides that have the ability to induce CTLs against cancerous cells are exemplified by cancer specific antigens (e.g., identified TAAs), but are not limited thereto.

If needed, the pharmaceutical compositions of the present invention can optionally include other therapeutic substances as an active ingredient, so long as the substance does not inhibit the antitumoral effect of the active ingredient, e.g., any of the present peptides. For example, formulations can include anti-inflammatory compositions, pain killers, chemotherapeutics, and the like. In addition to including other therapeutic substances in the medicament itself, the medicaments of the present invention can also be administered sequentially or concurrently with the one or more other pharmacologic compositions. The amounts of medicament and pharmacologic composition depend, for example, on what type of pharmacologic composition(s) is/are used, the disease being treated, and the scheduling and routes of administration.

It should be understood that in addition to the ingredients particularly mentioned herein, the pharmaceutical compositions of the present invention can include other compositions conventional in the art having regard to the type of formulation in question.

In one embodiment of the present invention, the present pharmaceutical compositions can be included in articles of manufacture and kits containing materials useful for treating the pathological conditions of the disease to be treated, e.g., cancer. The article of manufacture can include a container of any of the present pharmaceutical compositions with a label. Suitable containers include bottles, vials, and test tubes. The containers can be formed from a variety of materials, such as glass or plastic. The label on the container should indicate the composition is used for treating or prevention of one or more conditions of the disease. The label can also indicate directions for administration and so on.

In addition to the container described above, a kit including a pharmaceutical composition of the present invention can optionally further include a second container housing a pharmaceutically-acceptable diluent. It can further include other materials desirable from a commercial and user standpoint, including other buffers, diluents, filters, needles, syringes, and package inserts with instructions for use.

The pharmaceutical compositions can, if desired, be presented in a pack or dispenser device which can contain one or more unit dosage forms containing the active ingredient. The pack can, for example, include metal or plastic foil, such as a blister pack. The pack or dispenser device can be accompanied by instructions for administration.

(1) Pharmaceutical Compositions Containing the Peptides as the Active Ingredient

The peptides of this invention can be administered directly as a pharmaceutical composition, or if necessary formulated by conventional formulation methods. In the latter case, in addition to the peptides of this invention, carriers, excipients, and such that are ordinarily used for drugs can be included as appropriate without particular limitations. Examples of such carriers are sterilized water, physiological saline, phosphate buffer, culture fluid and such. Furthermore, the pharmaceutical compositions can contain as necessary, stabilizers, suspensions, preservatives, surfactants and such. The pharmaceutical compositions of the present invention can be used for anticancer purposes.

The peptides of the present invention can be prepared as a combination composed of two or more of peptides of the present invention, to induce CTL in vivo. The peptide combination can take the form of a cocktail or can be conjugated to each other using standard techniques. For example, the peptides can be chemically linked or expressed as a single fusion polypeptide sequence. The peptides in the combination can be the same or different. By administering the peptides of the present invention, the peptides are presented at a high density by the HLA antigens on APCs, then CTLs that specifically react toward the complex formed between the displayed peptide and the HLA antigen are induced. Alternatively, APCs (e.g., DCs) are removed from subjects and then stimulated by the peptides of the present invention to obtain APCs that present any of the peptides of the present invention on their cell surface. These APCs are readministered to the subjects to induce CTLs in the subjects, and as a result, aggressiveness towards the tumor-associated endothelium can be increased.

The pharmaceutical compositions for the treatment and/or prevention of cancer containing a peptide of the present invention as the active ingredient can also include an adjuvant known to effectively establish cellular immunity. Alternatively, the pharmaceutical compositions can be administered with other active ingredients, or administered by formulation into granules. An adjuvant refers to a compound that enhances the immune response against the protein when administered together (or successively) with the protein having immunological activity. Adjuvants contemplated herein include those described in the literature (Clin Microbiol Rev 1994, 7: 277-89). Examples of suitable adjuvants include, but are not limited to, aluminum phosphate, aluminum hydroxide, alum, cholera toxin, salmonella toxin, and the like.

Furthermore, liposome formulations, granular formulations in which the peptide is bound to few-micrometers diameter beads, and formulations in which a lipid is bound to the peptide may be conveniently used.

In another embodiment of the present invention, the peptides of the present invention may also be administered in the form of a pharmaceutically acceptable salt. Examples of preferred salts include salts with an alkali metal, salts with a metal, salts with an organic base, salts with an organic acid and salts with an inorganic acid.

In some embodiments, the pharmaceutical compositions of the present invention may further include a component that primes CTL. Lipids have been identified as compositions capable of priming CTL in vivo against viral antigens. For example, palmitic acid residues can be attached to the epsilon- and alpha-amino groups of a lysine residue and then linked to a peptide of the invention. The lipidated peptide can then be administered either directly in a micelle or particle, incorporated into a liposome, or emulsified in an adjuvant. As another example of lipid priming of CTL responses, E. coli lipoproteins, such as tripalmitoyl-S-glycerylcysteinyl-seryl-serine (P3CSS) can be used to prime CTL when covalently attached to an appropriate peptide (see, e.g., Deres et al., Nature 1989, 342: 561-4).

The method of administration can be oral, intradermal, subcutaneous, intravenous injection, or such, and systemic administration or local administration to the vicinity of the targeted sites. The administration can be performed by single administration or boosted by multiple administrations. The dose of the peptides of the present invention can be adjusted appropriately according to the disease to be treated, age of the patient, weight, method of administration, and such, and is ordinarily 0.001 mg to 1000 mg, for example, 0.001 mg to 1000 mg, for example, 0.1 mg to 10 mg, and can be administered once in a few days to few months. One skilled in the art can appropriately select a suitable dose.

(2) Pharmaceutical Compositions Containing Polynucleotides as the Active Ingredient

The pharmaceutical compositions of the invention can also contain nucleic acids encoding the peptides disclosed herein in an expressible form. Herein, the phrase “in an expressible form” means that the polynucleotide, when introduced into a cell, will be expressed in vivo as a polypeptide that induces anti-tumor immunity. In an exemplified embodiment, the nucleic acid sequence of the polynucleotide of interest includes regulatory elements necessary for expression of the polynucleotide. The polynucleotide(s) can be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687).

The peptides of the present invention can also be expressed by viral or bacterial vectors. Examples of expression vectors include attenuated viral hosts, such as vaccinia or fowlpox. This approach involves the use of vaccinia virus, e.g., as a vector to express nucleotide sequences that encode the peptide. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover et al., Nature 1991, 351: 456-60. A wide variety of other vectors useful for therapeutic administration or immunization e.g., adeno and adeno-associated virus vectors, retroviral vectors, Salmonella typhi vectors, detoxified anthrax toxin vectors, and the like, will be apparent. See, e.g., Shata et al., Mol Med Today 2000, 6: 66-71; Shedlock et al., J Leukoc Biol 2000, 68: 793-806; Hipp et al., In Vivo 2000, 14: 571-85.

Delivery of a polynucleotide into a patient can be either direct, in which case the patient is directly exposed to a polynucleotide-carrying vector, or indirect, in which case, cells are first transformed with the polynucleotide of interest in vitro, then the cells are transplanted into the patient. Theses two approaches are known, respectively, as in vivo and ex vivo gene therapies.

For general reviews of the methods of gene therapy, see Goldspiel et al., Clinical Pharmacy 1993, 12: 488-505; Wu and Wu, Biotherapy 1991, 3: 87-95; Tolstoshev, Ann Rev Pharmacol Toxicol 1993, 33: 573-96; Mulligan, Science 1993, 260: 926-32; Morgan & Anderson, Ann Rev Biochem 1993, 62: 191-217; Trends in Biotechnology 1993, 11(5): 155-215). Methods commonly known in the art of recombinant DNA technology which can also be used for the present invention are described in eds. Ausubel et al., Current Protocols in Molecular Biology, John Wiley & Sons, NY, 1993; and Krieger, Gene Transfer and Expression, A Laboratory Manual, Stockton Press, NY, 1990.

The method of administration can be oral, intradermal, subcutaneous, intravenous injection, or such, and systemic administration or local administration to the vicinity of the targeted sites finds use. The administration can be performed by single administration or boosted by multiple administrations. The dose of the polynucleotide in the suitable carrier or cells transformed with the polynucleotide encoding the peptides of the present invention can be adjusted appropriately according to the disease to be treated, age of the patient, weight, method of administration, and such, and is ordinarily 0.001 mg to 1000 mg, for example, 0.001 mg to 1000 mg, for example, 0.1 mg to 10 mg, and can be administered once every a few days to once every few months. One skilled in the art can appropriately select the suitable dose.

### X. Methods Using the Peptides, Exosomes, APCs and CTLs

The peptides and polynucleotides of the present invention can be used for inducing APCs and CTLs. The exosomes and APCs of the present invention can be also used for inducing CTLs. The peptides, polynucleotides, exosomes and APCs can be used in combination with any other compounds so long as the compounds do not inhibit their CTL inducibility. Thus, any of the aforementioned pharmaceutical compositions of the present invention can be used for inducing CTLs, and in addition thereto, those including the peptides and polynucleotides can be also be used for inducing APCs as discussed below.

(1) Method of Inducing Antigen-Presenting Cells (APCs)

The present invention provides methods of inducing APCs with high CTL inducibility using the peptides or polynucleotides of the present invention.

The methods of the present invention include the step of contacting APCs with the peptides of the present invention in vitro, ex vivo or in vivo. For example, the method contacting APCs with the peptides ex vivo can include steps of:

a: collecting APCs from a subject, and

b: contacting the APCs of step a with the peptide.

The APCs are not limited to a particular kind of cells and include DCs, Langerhans cells, macrophages, B cells, and activated T cells, which are known to present proteinaceous antigens on their cell surface so as to be recognized by lymphocytes. Preferably, DCs can be used since they have the strongest CTL inducibility among APCs. Any peptides of the present invention can be used by themselves or with other peptides of the present invention.

On the other hands, when the peptides of the present invention are administered to a subject, the APCs are contacted with the peptides in vivo, consequently, the APCs with high CTL inducibility are induced in the body of the subject. Thus, the present invention includes administering the peptides of the present invention to a subject. Similarly, when the polynucleotides of this invention are administered to a subject in an expressible form, the peptides of the present invention are expressed and contacted with APCs in vivo, consequently, the APCs with high CTL inducibility are induced in the body of the subject. Thus, the present invention also includes administering the polynucleotides of the present invention to a subject. “Expressible form” was described above in section “IX. Pharmaceutical compositions (2) Pharmaceutical compositions containing polynucleotides as the active ingredient”

The present invention also includes introducing the polynucleotide of the present invention into an APCs to induce APCs with CTL inducibility. For example, the method can include steps of:

a: collecting APCs from a subject, and

b: introducing a polynucleotide encoding peptide of the present invention.

Step b can be performed as described above in section “VI. Antigen-presenting cells”.

(2) Method of Inducing CTLs

The present invention also provides methods for inducing CTLs using the peptides, polynucleotides, or exosomes or APCs of the present invention.

When the peptides, the polynucleotides, APCs, or exosomes of the present invention are administered to a subject, CTL is induced in the body of the subject, and the strength of the immune response targeting the cancer cells is enhanced. Thus, the methods of the present invention includes the step of administering the peptides, the polynucleotides, the APCs or exosomes of the present invention to a subject.

Alternatively, CTL can be also induced by using them ex vivo, and after inducing CTL, the activated CTLs are returned to the subject. For example, the method can include steps of:

a: collecting APCs from subject,

b: contacting with the APCs of step a, with the peptide, and

c: co-culturing the APCs of step b with CD8 positive cells.

The APCs to be co-cultured with the CD8 positive cells in above step c can also be prepared by transferring a gene that includes a polynucleotide of the present invention into APCs as described above in section “VI. Antigen-presenting cells”, though the present invention is not limited thereto and encompasses any APCs that effectively present on its surface a complex of an HLA antigen and a peptide of the present invention.

Instead of such APCs, the exosomes that presents on its surface a complex of an HLA antigen and the peptide of the present invention can be also used. Namely, the present invention can includes the step of co-culturing exosomes presenting on its surface a complex of an HLA antigen and the peptide of the present invention. Such exosomes can be prepared by the methods described above in section “V. Exosomes”.

Furthermore, CTL can be induced by introducing a gene that includes a polynucleotide encoding the TCR subunit binding to the peptide of the present invention into CD8 positive cells. Such transduction can be performed as described above in section “VIII. T cell receptor (TCR)”.

In addition, the present invention provides a method or process for manufacturing a pharmaceutical composition inducing CTLs, wherein the method includes the step of admixing or formulating the peptide of the present invention with a pharmaceutically acceptable carrier.

### XI. Methods for Detecting or Diagnosing Cancer

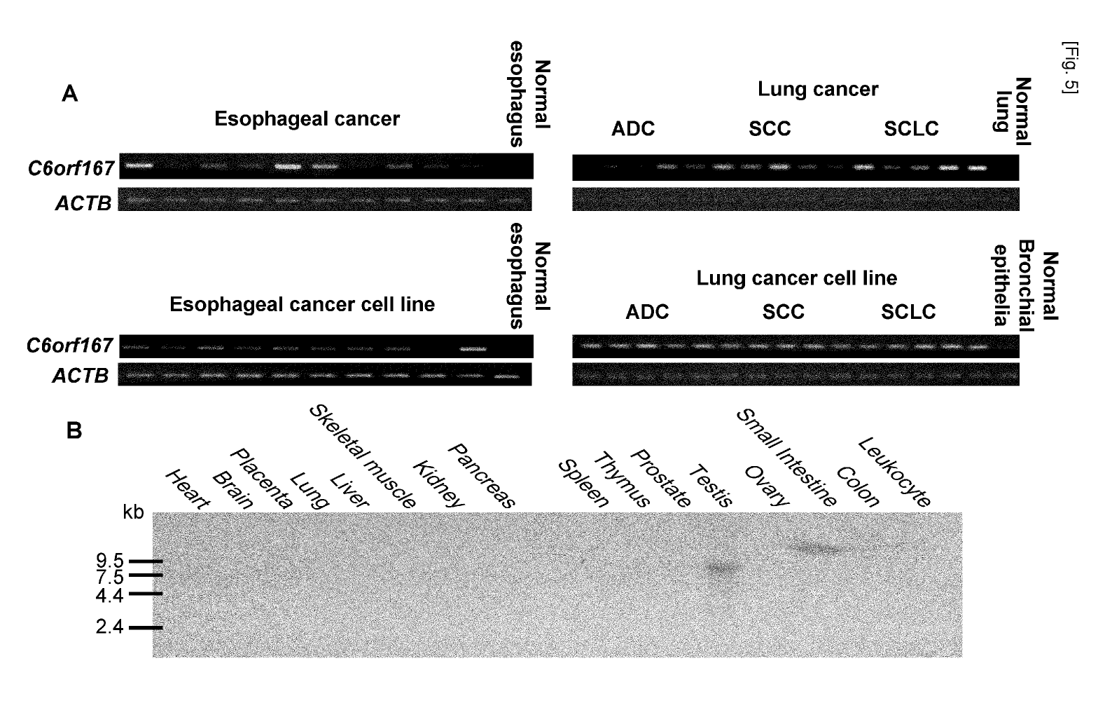

The present invention also provides a method of detecting or diagnosing cancer. The expression of C6orf167 was found to be specifically elevated in several kinds of cancer cell lines and clinical cancer tissues (Table 1 and FIG. 5). Therefore, the genes identified herein as well as their transcription and translation products find diagnostic utility as markers for cancer and by measuring the expression of C6orf167 in a cell sample.

Specifically, the present invention provides a method for detecting the presence of cancer in a biological sample or a subject from that the biological sample is derived, by determining the expression level of C6orf167 in the biological sample and comparing the determined expression level with a control level determined in a sample known to be cancerous (hereinafter referred to as “cancer control”) or not cancerous (hereinafter referred to as “normal control”). Cancers to be detected by the methods and compositions of the present method can be any cancers relating to C6orf167 up-regulation. Examples of such cancers include, but are not limited to, bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor.

In another aspect, the present invention also provides a method for diagnosing cancer in a subject. The diagnosis may be conducted on the basis of the result obtained by the method for detecting cancer described above.

According to the present invention, an intermediate result for examining the condition of a subject may be provided. Such intermediate result may be combined with additional information to assist a doctor, nurse, or other practitioner to diagnose that a subject may suffer from the disease. Alternatively, the present invention may be used to detect cancerous cells in a subject-derived tissue, and provide a doctor with useful information to diagnose that the subject suffers from the disease.

Specifically, the present invention provides the following methods [1] to [10]:

[1] A method for detecting or diagnosing cancer, the method including the steps of:

(a) determining the expression level of the gene encoding the amino acid sequence of C6orf167 in a biological sample; and

(b) correlating an increase in the expression level determined as compared to a normal control level of the gene to the presence of disease.

[2] The method of [1], wherein the expression level is at least 10% greater than the normal control level.

[3] The method of [1], wherein the expression level is determined by a method selected from among:

(a) detecting an mRNA of the C6orf167 gene,

(b) detecting a protein encoded by the C6orf167 gene, and

(c) detecting a biological activity of a protein encoded by the C6orf167 gene.

[4] The method of [1], wherein the cancer is selected from group of bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor.

[5] The method of [3], wherein the expression level is determined by detecting hybridization of a probe to a gene transcript of the gene.

[6] The method of [3], wherein the expression level is determined by detecting the binding of an antibody against the protein encoded by a gene as the expression level of the gene.

[7] The method of [1], wherein the biological sample includes biopsy, sputum, blood, pleural effusion or urine.

[8] The method of [1], wherein the subject-derived biological sample includes an epithelial cell.

[9] The method of [1], wherein the subject-derived biological sample includes a cancer cell.

[10] The method of [1], wherein the subject-derived biological sample includes a cancerous epithelial cell.

Alternatively, the present invention provides a method for detecting or identifying cancer cells in a subject-derived bladder tissue, cervical tissue, bileduct tissue (intrahepatic bile duct tissue), leukocyte, esophageal tissue, gastric tissue, lung tissue, lymphatic tissue, bone tissue, renal tissue, lung tissue, soft tissue, or testicular tissue sample, the method comprising the step of determining the expression level of the C6orf167 gene in a subject-derived biological sample, wherein an increase in the expression level as compared to a normal control level of the gene indicates the presence or suspicion of cancer cells in the tissue.

Such result may be combined with additional information to assist a doctor, nurse, or other healthcare practitioner in diagnosing a subject as afflicted with the disease. In other words, the present invention may provide a doctor with useful information to diagnose a subject as afflicted with the disease. For example, according to the present invention, when there is doubt regarding the presence of cancer cells in the tissue obtained from a subject, clinical decisions can be reached by considering the expression level of the C6orf167 gene, plus a different aspect of the disease including tissue pathology, levels of known tumor marker(s) in blood, and clinical course of the subject, etc. For example, some well-known diagnostic tumor markers in blood are as follows.

bladder cancer;

IAP, SCC, NMP, BFP, and TPA

cervical cancer;

SCC, CEA, CYFRA21-1, CEA, or CA125

cholangiocellular carcinoma;

CEA, or CA19-9

chronic myelogenous leukemia (CML);

TK activity

esophageal cancer;

CEA, DUPAN-2, IAP, NSE, SCC, SLX, or Span-1

gastric cancer;

CEA, SLX, STN, or NCC-ST-439

gastric diffuse-type cancer;

CEA, SLX, STN, NCC-ST-439, hsCRP or PG I/II

lung cancer;

AP, ACT, BFP, CA19-9, CA50, CA72-4, CA130, CEA, KMO-1, NSE, SCC, SP1,

Span-1, TPA, CSLEX, SLX, STN or CYFRA

lymphoma;

IAP, Span-1, or TPA

osteosarcoma;

CD44, or ALP

renal carcinoma;

BFP, or IAP

lung adenocarcinoma (ADC);

NCC-ST-439, CEA, or SLX

lung squamous cell carcinoma (SCC);

SCC, or Cyfra

small-cell lung cancer (SCLC);

Pro-GRP, or NSE

non-small-cell lung cancer (NSCLC);

NCC-ST-439, CEA, SLX, SCC, or Cyfra

testicular tumor;

AFP, or BFP

Namely, in this particular embodiment of the present invention, the outcome of the gene expression analysis serves as an intermediate result for further diagnosis of a subject's disease state.

In another embodiment, the present invention provides a method for detecting a diagnostic marker of cancer, the method comprising the step of detecting the expression of the C6orf167 gene in a subject-derived biological sample as a diagnostic marker of bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor.

The method of detecting or diagnosing cancer will be described in more detail below.

A subject to be diagnosed by the present method is preferably a mammal. Exemplary mammals include, but are not limited to, e.g., human, non-human primate, mouse, rat, dog, cat, horse, and cow.

It is preferred to collect a biological sample from the subject to be diagnosed to perform the diagnosis. Any biological material can be used as the biological sample for the determination so long as it includes the objective transcription or translation product of C6orf167. The biological samples include, but are not limited to, bodily tissues and fluids, such as blood, sputum and urine. Preferably, the biological sample contains a cell population including an epithelial cell, more preferably a cancerous epithelial cell or an epithelial cell derived from tissue suspected to be cancerous. Further, if necessary, the cell may be purified from the obtained bodily tissues and fluids, and then used as the biological sample.

According to the present invention, the expression level of C6orf167 in the subject-derived biological sample is determined. The expression level can be determined at the transcription product (i.e., mRNA) level, using methods known in the art. For example, the mRNA of C6orf167 may be quantified using probes by hybridization methods (e.g., Northern hybridization). The detection may be carried out on a chip or an array. The use of an array is preferable for detecting the expression level of a plurality of genes (e.g., various cancer specific genes) including the C6orf167 gene. Those skilled in the art can prepare such probes utilizing the sequence information of the C6orf167 gene (SEQ ID NO: 158; GenBank accession number: NM_{—}198468.2). For example, the cDNA of C6orf167 may be used as the probes. If necessary, the probe may be labeled with a suitable label, such as dyes, fluorescent and isotopes, and the expression level of the gene may be detected as the intensity of the hybridized labels.

Furthermore, the transcription product of the C6orf167 gene may be quantified using primers by amplification-based detection methods (e.g., RT-PCR). Such primers can also be prepared based on the available sequence information of the gene. For example, the primers (SEQ ID NOs: 154, 155, 156 and 157) used in “Examples” may be employed for the detection by RT-PCR or Northern blot, but the present invention is not restricted thereto.

Specifically, a probe or primer used for the present method hybridizes under stringent, moderately stringent, or low stringent conditions to the mRNA of C6orf167. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degree Centigrade lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degree Centigrade for short probes or primers (e.g., 10 to 50 nucleotides) and at least about 60 degree Centigrade for longer probes or primers. Stringent conditions may also be achieved with the addition of destabilizing compositions, such as formamide.

Alternatively, the translation product (i.e., protein) may be detected for the diagnosis of the present invention. For example, the quantity of C6orf167 protein may be determined. A method for determining the quantity of the protein as the translation product includes immunoassay methods that use an antibody specifically recognizing the protein. The antibody may be monoclonal or polyclonal. Furthermore, any fragment or modification (e.g., chimeric antibody, scFv, Fab, F(ab′)₂, Fv, etc.) of the antibody may be used for the detection, so long as the fragment retains the binding ability to C6orf167 protein. Methods to prepare these kinds of antibodies for the detection of proteins are well known in the art, and any method may be employed in the present invention to prepare such antibodies and equivalents thereof.

As another method to detect the expression level of C6orf167 gene based on its translation product, the intensity of staining may be observed via immunohistochemical analysis using an antibody against C6orf167 protein. Namely, the observation of strong staining indicates increased presence of the protein and at the same time high expression level of C6orf167 gene.

Furthermore, the translation product may be detected based on its biological activity. For example, the C6orf167 protein appears to be involved in the proliferation of cancer cells. Thus, the cell proliferative activity of the C6orf167 protein may be used as an index of the C6orf167 protein existing in the biological sample.

Moreover, in addition to the expression level of C6orf167 gene, the expression level of other cancer-associated genes, for example, genes known to be differentially expressed in cancer may also be assayed to improve the accuracy of the diagnosis.

The expression level of cancer marker genes, including the C6orf167 gene, in a biological sample is considered to be “increased” if it is above the control level of the corresponding cancer marker gene by, for example, 10%, 25%, or 50%; or increases to more than 1.1 fold, more than 1.5 fold, more than 2.0 fold, more than 5.0 fold, more than 10.0 fold, or more.

The control level may be determined at the same time as the test biological sample or by using a sample(s) previously collected and stored from a subject/subjects whose disease state (cancerous or non-cancerous) is/are known. Alternatively, the control level may be determined by a statistical method based on the results obtained by analyzing previously determined expression level(s) of C6orf167 gene in samples from subjects whose disease state is known. Furthermore, the control level can be a database of expression patterns from previously tested cells. Moreover, according to an aspect of the present invention, the expression level of C6orf167 gene in a biological sample may be compared to multiple control levels, determined from multiple reference samples. It is preferred to use a control level determined from a reference sample derived from a tissue type similar to that of the subject-derived biological sample. Moreover, it is preferred, to use the standard value of the expression levels of C6orf167 gene in a population with a known disease state. The standard value may be obtained by any method known in the art. For example, a range of mean+/−2 S.D. or mean+/−3 S.D. may be used as standard value.

In the context of the present invention, a control level determined from a biological sample that is known not to be cancerous is referred to as a “normal control level”. On the other hand, a control level is determined from a cancerous biological sample is referred to as a “cancerous control level”.

When the expression level of C6orf167 gene is increased as compared to the normal control level or is similar to the cancerous control level, the subject may be diagnosed to be suffering from or at a risk of developing cancer. Furthermore, when the expression levels of multiple cancer-related genes are compared, a similarity in the gene expression pattern between the sample and the reference which is cancerous indicates that the subject is suffering from or at a risk of developing cancer.

Difference between the expression levels of a test biological sample and the control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

### XII. Kits for Detecting or Diagnosing Cancer

The present invention also provides a diagnostic kit for diagnosing or determining a subject who is or is suspected to be suffering from cancer that can be treated with the C6orf167 polypeptide of the present invention, which may also find use in assessing the prognosis of cancer and/or monitoring efficacy or applicability of a cancer therapy, particularly a cancer immunotherapy. Illustrative example of cancers to be diagnosed or determined include, but are not limited to, bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor. More particularly, the kit preferably may include at least one reagent for detecting the expression of the C6orf167 gene in a subject-derived cell, such reagent selected from the group of:

(a) a reagent for detecting mRNA of the C6orf167 gene;

(b) a reagent for detecting the C6orf167 protein or the immunologically fragment thereof; and

(c) a reagent for detecting the biological activity of the C6orf167 protein.

Examples of reagents suitable for detecting the C6orf167 mRNA include nucleic acids that specifically bind to or identify the C6orf167 mRNA, such as oligonucleotides that have a complementary sequence to a part of the C6orf167 mRNA. These kinds of oligonucleotides are exemplified by primers and probes that are specific to the C6orf167 mRNA. These kinds of oligonucleotides can be prepared based on methods well known in the art. If needed, the reagent for detecting the C6orf167 mRNA can be immobilized on a solid matrix. Moreover, more than one reagent for detecting the C6orf167 mRNA can be included in the kit.

On the other hand, examples of reagents suitable for detecting the C6orf167 protein include antibodies to the C6orf167 protein. The antibody can be monoclonal or polyclonal. Furthermore, any fragment or modification (e.g., chimeric antibody, scFv, Fab, F(ab′)₂, Fv, etc.) of the antibody can be used as the reagent, so long as the fragment retains the binding ability to the C6orf167 protein. Methods to prepare these kinds of antibodies for the detection of proteins are well known in the art, and any method can be employed in the present invention to prepare such antibodies and equivalents thereof. Furthermore, the antibody can be labeled with signal generating molecules via direct linkage or an indirect labeling technique. Labels and methods for labeling antibodies and detecting the binding of antibodies to their targets are well known in the art and any labels and methods can be employed for the present invention. Moreover, more than one reagent for detecting the C6orf167 protein can be included in the kit.

Furthermore, the biological activity can be determined by, for example, measuring the cell proliferating activity due to the expressed the C6orf167 protein in the biological sample. For example, the cell may be cultured in the presence of a biological sample, and then the cell proliferating activity of the biological sample can be assayed, for example by detecting the speed of proliferation, or by measuring the cell cycle or the colony forming ability. If necessary, the reagent for detecting the C6orf167 mRNA can be immobilized on a solid matrix. Moreover, more than one reagent for detecting the biological activity of the C6orf167 protein can be included in the kit.

The kit can include more than one of the aforementioned reagents. The kit can further include a solid matrix and reagent for binding a probe against the C6orf167 gene or antibody against the C6orf167 protein, a medium and container for culturing cells, positive and negative control reagents, and a secondary antibody for detecting an antibody against the C6orf167 protein. For example, tissue samples obtained from a subject known to be non-cancerous can serve as useful control reagents. A kit of the present invention can further include other materials desirable from a commercial and user standpoint, including buffers, diluents, filters, needles, syringes, and package inserts (e.g., written, tape, CD-ROM, etc.) with instructions for use. These reagents and such can be retained in a container with a label. Suitable containers include bottles, vials, and test tubes. The containers can be formed from a variety of materials, for example, glass or plastic.

As an embodiment of the present invention, when the reagent is a probe against the C6orf167 mRNA, the reagent can be immobilized on a solid matrix, for example, a porous strip, to form at least one detection site. The measurement or detection region of the porous strip can include a plurality of sites, each containing a nucleic acid (probe). A test strip can also contain sites for negative and/or positive controls. Alternatively, control sites can be located on a strip separated from the test strip. Optionally, the different detection sites can contain different amounts of immobilized nucleic acids, i.e., a higher amount in the first detection site and lesser amounts in subsequent sites. Upon the addition of test sample, the number of sites displaying a detectable signal provides a quantitative indication of the amount of C6orf167 mRNA present in the sample. The detection sites can be configured in any suitably detectable shape and are typically in the shape of a bar or dot spanning the width of a test strip.

In further embodiment, the present invention further provides a diagnostic kit including, a protein or a partial protein thereof capable of specifically recognizing the antibody of the present invention or an immunogenic fragment thereof.

Examples of partial peptides and immunogenic fragments of the proteins of the present invention contemplated herein include polypeptides composed of at least 8, preferably 15, and more preferably 20 contiguous amino acids in the amino acid sequence of the protein of the present invention. Cancer can be diagnosed by detecting an antibody in a sample (e.g., blood, tissue) using a protein or a peptide (polypeptide) of the present invention. Methods for preparing a peptide or protein of the present invention are as described above.

The method for diagnosing cancer of the present invention can be performed by determining the difference between the amount of anti-C6orf167 antibody and that in the corresponding control sample as describe above. The subject is suspected to be suffering from cancer, if biological samples of the subject contain antibodies against the expression products (C6orf167) of the gene and the quantity of the anti-C6orf167 antibody is determined to be more than the cut off value in level compared to that in normal control.

In another embodiment, a diagnostic kit of the present invention may include the peptide of the present invention and an HLA molecule binding thereto. A suitable method for detecting antigen specific CTLs using antigenic peptides and HLA molecules has already been established (for example, Altman J D et al., Science. 1996, 274(5284): 94-6). Thus, the complex of the peptide of the present invention and the HLA molecule can be applied to the detection method to detect tumor antigen specific CTLs, thereby enabling earlier detection, recurrence and/or metastasis of cancer. Further, it can be employed for the selection of subjects applicable with the pharmaceuticals including the peptide of the present invention as an active ingredient, or the assessment of the treatment effect of the pharmaceuticals.

Particularly, according to the known method (see, for example, Altman J D et al., Science. 1996, 274(5284): 94-6), the oligomer complex, such as tetramer, of the radiolabeled HLA molecule and the peptide of the present invention can be prepared. The complex may be used to quantify the antigen-peptide specific CTLs in the peripheral blood lymphocytes derived from the subject suspected to be suffering from cancer.

The present invention further provides methods and diagnostic agents for evaluating the immunological response of subject using peptide epitopes as described herein. In one embodiment of the invention, HLA restricted peptides as described herein may be used as reagents for evaluating or predicting an immune response of a subject. The immune response to be evaluated may be induced by contacting an immunogen with immunocompetent cells in vitro or in vivo. In certain embodiments, the composition employed as the reagent may be any composition that may result in the production of antigen specific CTLs that recognize and bind to the peptide epitope(s). The peptide reagents need not be used as the immunogen. Assay systems that are used for such an analysis include relatively recent technical developments such as tetramers, staining for intracellular lymphokines and interferon release assays, or ELISPOT assays. In a preferred embodiment, immunocompetent cells to be contacted with peptide reagent may be antigen presenting cells including dendritic cells.

For example, peptides of the present invention may be used in tetramer staining assays to assess peripheral blood mononuclear cells for the presence of antigen-specific CTLs following exposure to a tumor cell antigen or an immunogen. The HLA tetrameric complex may be used to directly visualize antigen specific CTLs (see, e.g., Ogg et al., Science 279: 2103-2106, 1998; and Altman et al, Science 174: 94-96, 1996) and determine the frequency of the antigen-specific CTL population in a sample of peripheral blood mononuclear cells. A tetramer reagent using a peptide of the invention may be generated as described below.

A peptide that binds to an HLA molecule is refolded in the presence of the corresponding HLA heavy chain and beta 2-microglobulin to generate a trimolecular complex. In the complex, carboxyl terminal of the heavy chain is biotinylated at a site that was previously engineered into the protein. Then, streptavidin is added to the complex to form tetramer consisting of the trimolecular complex and streptavidin. By means of fluorescently labeled streptavidin, the tetramer can be used to stain antigen specific cells. The cells can then be identified, for example, by flow cytometry. Such an analysis may be used for diagnostic or prognostic purposes. Cells identified by the procedure can also be used for therapeutic purposes.

The present invention also provides reagents to evaluate immune recall responses (see, e.g., Bertoni et al, J. Clin. Invest. 100: 503-513, 1997 and Penna et al., J. Exp. Med. 174: 1565-1570, 1991) including peptides of the present invention. For example, patient PBMC samples from individuals with cancer to be treated can be analyzed for the presence of antigen-specific CTLs using specific peptides. A blood sample containing mononuclear cells can be evaluated by cultivating the PBMCs and stimulating the cells with a peptide of the invention. After an appropriate cultivation period, the expanded cell population can be analyzed, for example, for CTL activity.

The peptides may also be used as reagents to evaluate the efficacy of a vaccine. PBMCs obtained from a patient vaccinated with an immunogen may be analyzed using, for example, either of the methods described above. The patient is HLA typed, and peptide epitope reagents that recognize the allele specific molecules present in the patient are selected for the analysis. The immunogenicity of the vaccine may be indicated by the presence of epitope-specific CTLs in the PBMC sample. The peptides of the invention may also be used to make antibodies, using techniques well known in the art (see, e.g., CURRENTPROTOCOLSINIMMUNOLOGY, Wiley/Greene, NY; and Antibodies A Laboratory Manual, Harlow and Lane, Cold Spring Harbor Laboratory Press, 1989), which may find use as reagents to diagnose, detect or monitor cancer. Such antibodies may include those that recognize a peptide in the context of an HLA molecule, i.e., antibodies that bind to a peptide-MHC complex.

The peptides and compositions of the present invention have a number of additional uses, some of which are described herein. For instance, the present invention provides a method for diagnosing or detecting a disorder characterized by expression or presentation of a C6orf167 immunogenic polypeptide. Such methods involve determining expression or presentation of a C6orf167 HLA binding peptide, or a complex of a C6orf167 HLA binding peptide and an HLA class I molecule in a biological sample. The expression or presentation of a peptide or complex of peptide and HLA class I molecule can be determined or detected by assaying with a binding partner for the peptide or complex. In a preferred embodiment, a binding partner for the peptide or complex may be an antibody recognizes and specifically bind to the peptide or the complex. The expression of C6orf167 in a biological sample, such as a tumor biopsy, can also be tested by standard PCR amplification protocols using C6orf167 primers. An example of tumor expression is presented herein and further disclosure of exemplary conditions and primers for C6orf167 amplification can be found in WO2003/27322.

Preferred diagnostic methods involve contacting a biological sample isolated from a subject with a substance specific for the C6orf167 HLA binding peptide to detect the presence of the C6orf167 HLA binding peptide in the biological sample. As used herein, “contacting” means placing the biological sample in sufficient proximity to the agent and under the appropriate conditions of, e.g., concentration, temperature, time, ionic strength, to allow the specific interaction between the agent and C6orf167 HLA binding peptide that are present in the biological sample. In general, the conditions for contacting the agent with the biological sample are conditions known by those of ordinary skill in the art to facilitate a specific interaction between a molecule and its cognate (e.g., a protein and its receptor cognate, an antibody and its protein antigen cognate, a nucleic acid and its complementary sequence cognate) in a biological sample. Exemplary conditions for facilitating a specific interaction between a molecule and its cognate are described in U.S. Pat. No. 5,108,921, issued to Low et al.

The diagnostic methods of the present invention can be performed in either or both of in vivo and in vitro. Accordingly, biological sample can be located in vivo or in vitro in the present invention. For example, the biological sample can be a tissue in vivo and the agent specific for the C6orf167 immunogenic polypeptide can be used to detect the presence of such molecules in the tissue. Alternatively, the biological sample can be collected or isolated in vitro (e.g., a blood sample, tumor biopsy, tissue extract). In a particularly preferred embodiment, the biological sample can be a cell-containing sample, more preferably a sample containing tumor cells collected from a subject to be diagnosed or treated.

Alternatively, the diagnosis can be performed using a method that allows direct quantification of antigen-specific T cells by staining with Fluorescein-labeled HLA multimeric complexes (e.g., Altman, J. D. et al., 1996, Science 274: 94; Altman, J. D. et al., 1993, Proc. Natl. Acad. Sci. USA 90: 10330). Staining for intracellular lymphokines, and interferon-gamma release assays or ELISPOT assays also has been provided. Multimer staining, intracellular lymphokine staining and ELISPOT assays all appear to be at least 10-fold more sensitive than more conventional assays (Murali-Krishna, K. et al., 1998, Immunity 8: 177; Lalvani, A. et al., 1997, J. Exp. Med. 186: 859; Dunbar, P. R. et al., 1998, Curr. Biol. 8: 413). Pentamers (e.g., US 2004-209295A), dextramers (e.g., WO 02/072631), and streptamers (e.g., Nature medicine 6, 631-637 (2002)) may also be used.

### XIII. Methods of Inducing Immune Response

Moreover, the present invention provides methods of inducing immune response against diseases related to C6orf167. Suitable diseases include cancer, examples of which include, but are not limited to, bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor.

The methods of the present invention may include the step of administering substance(s) or composition(s) containing any of the peptides of the present invention or polynucleotides encoding them. The inventive methods also contemplate the administration of exosomes or APCs presenting any of the peptides of the present invention. For details, see the item of “IX. Pharmaceutical compositions”, particularly the part describing the use of the pharmaceutical compositions of the present invention as vaccines. In addition, the exosomes and APCs that can be employed for the present methods for inducing immune response are described in detail under the items of “V. Exosomes”, “VI. Antigen-presenting cells (APCs)”, and (1) and (2) of “X. Methods using the peptides, exosomes, APCs and CTLs”, supra.

The present invention also provides a method or process for manufacturing a pharmaceutical composition or substance inducing immune response, wherein the method may include the step of admixing or formulating the peptide of the present invention with a pharmaceutically acceptable carrier.

Alternatively, the method of the present invention may include the step of administrating a vaccine or a pharmaceutical composition or substance that contains:

(a) a peptide of the present invention;

(b) a nucleic acid encoding such a peptide as disclosed herein in an expressible form;

(c) an APC or an exosome presenting a peptide of the present invention on its surface; or

(d) a cytotoxic T cell of the present invention.

In the context of the present invention, a cancer over-expressing C6orf167 can be treated with these active ingredients. Examples of such cancer include, but are not limited to, bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor. Accordingly, prior to the administration of the vaccines or pharmaceutical compositions or substance including the active ingredients, it is preferable to confirm whether the expression level of C6orf167 in the subject to be treated is enhanced. Thus, in one embodiment, the present invention provides a method for treating cancer (over)expressing C6orf167 in a patient in need thereof, such method including the steps of:

i) determining the expression level of C6orf167 in biological sample(s) obtained from a subject with the cancer to be treated;
ii) comparing the expression level of C6orf167 with normal control; and
iii) administrating at least one component selected from the group consisting of (a) to

(d) described above to a subject with cancer overexpressing C6orf167 compared with normal control.

Alternatively, the present invention also provides a vaccine or pharmaceutical composition or substance including at least one component selected from the group consisting of (a) to (d) described above, to be administered to a subject having cancer overexpressing C6orf167. In other words, the present invention further provides a method for identifying a subject to be treated with the C6orf167 polypeptide of the present invention, which method may include the step of determining an expression level of C6orf167 in subject-derived biological sample(s), wherein an increase of the level compared to a normal control level of the gene indicates that the subject may have cancer which may be treated with the C6orf167 polypeptide of the present invention. The method of treating cancer of the present invention will be described in more detail in below.

According to the present invention, the expression level of C6orf167 in biological sample obtained from a subject may be determined. The expression level can be determined, for example, according to methods described in “XI. A method for detecting or diagnosing cancer”, supra.

In one embodiment, the present invention provides a method of (i) diagnosing whether a subject suspected to have cancer to be treated, and/or (ii) selecting a subject for cancer treatment, such method including the steps of:

a) determining the expression level of C6orf167 in biological sample(s) obtained from a subject who is suspected to have the cancer to be treated;

b) comparing the expression level of C6orf167 with a normal control level;

c) diagnosing the subject as having the cancer to be treated, if the expression level of C6orf167 is increased as compared to the normal control level; and

d) selecting the subject for cancer treatment, if the subject is diagnosed as having the cancer to be treated, in step c).

Alternatively, such a method may include the steps of:

a) determining the expression level of C6orf167 in biological sample(s) obtained from a subject who is suspected to have the cancer to be treated;

b) comparing the expression level of C6orf167 with a cancerous control level;

c) diagnosing the subject as having the cancer to be treated, if the expression level of C6orf167 is similar or equivalent to the cancerous control level; and

d) selecting the subject for cancer treatment, if the subject is diagnosed as having the cancer to be treated, in step c).

### XIV. Antibodies

The present invention further provides antibodies that bind to peptides of the present invention. Preferred antibodies specifically bind to peptides of the present invention and will not bind (or will bind weakly) to non-peptide of the present invention. Alternatively, antibodies bind to peptides of the invention as well as the homologs thereof. Antibodies against peptides of the invention can find use in cancer diagnostic and prognostic assays, and imaging methodologies. Similarly, such antibodies can find use in the treatment, diagnosis, and/or prognosis of other cancers, to the extent C6orf167 is also expressed or overexpressed in cancer patient. Moreover, intracellularly expressed antibodies (e.g., single chain antibodies) may therapeutically find use in treating cancers in which the expression of C6orf167 is involved, example of which include, but are not limited to, bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor.

The present invention also provides various immunological assays for the detection and/or quantification of the C6orf167 protein (SEQ ID NO: 159) or fragments thereof, including polypeptides consisting of amino acid sequences selected from the group consisting of SEQ ID NOs: 1 to 61 and 63 to 151. Such assays may include one or more anti-C6orf167 antibodies capable of recognizing and binding a C6orf167 protein or fragments thereof, as appropriate. In the context of the present invention, anti-C6orf167 antibodies binding to C6orf167 polypeptide preferably recognize polypeptide consisting of amino acid sequences selected from the group consisting of SEQ ID NOs: 1 to 61 and 63 to 151. A binding specificity of antibody can be confirmed with inhibition test. That is, when the binding between an antibody to be analyzed and full-length of C6orf167 polypeptide is inhibited under presence of any fragment polypeptides consisting of amino acid sequence of SEQ ID NOs: 1 to 61 and 63 to 151, it is shown that this antibody specifically binds to the fragment. In the context of the present invention, such immunological assays are performed within various immunological assay formats well known in the art, including but not limited to, various types of radioimmunoassays, immuno-chromatograph technique, enzyme-linked immunosorbent assays (ELISA), enzyme-linked immunofluorescent assays (ELIFA), and the like.

Related immunological but non-antibody assays of the invention may also include T cell immunogenicity assays (inhibitory or stimulatory) as well as MHC binding assays. In addition, the present invention contemplates immunological imaging methods capable of detecting cancers expressing C6orf167, example of which include, but are not limited to, radioscintigraphic imaging methods using labeled antibodies of the present invention. Such assays find clinical use in the detection, monitoring, and prognosis of C6orf167 expressing cancers, examples of which include, but are not limited to, bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor.

The present invention also provides antibodies that bind to the peptides of the invention. An antibody of the invention can be used in any form, for example as a monoclonal or polyclonal antibody, and may further include antiserum obtained by immunizing an animal such as a rabbit with the peptide of the invention, all classes of polyclonal and monoclonal antibodies, human antibodies and humanized antibodies produced by genetic recombination.

A peptide of the invention used as an antigen to obtain an antibody may be derived from any animal species, but is preferably derived from a mammal such as a human, mouse, or rat, more preferably from a human. A human-derived peptide may be obtained from the nucleotide or amino acid sequences disclosed herein.

According to the present invention, the peptide to be used as an immunization antigen may be a complete protein or a partial peptide of the protein. A partial peptide may include, for example, the amino (N)-terminal or carboxy (C)-terminal fragment of a peptide of the present invention.

Herein, an antibody is defined as a protein that reacts with either the full length or a fragment of a C6orf167 peptide. In a preferred embodiment, an antibody of the present invention can recognize fragment peptides of C6orf167 consisting of amino acid sequence selected from the group consisting of SEQ ID NOs: 1-61 and 63 to 151. Methods for synthesizing oligopeptide are well known in the arts. After the synthesis, peptides may be optionally purified prior to use as immunogen. In the context of the present invention, the oligopeptide (e.g., 9- or 10mer) may be conjugated or linked with carriers to enhance the immunogenicity. Keyhole-limpet hemocyanin (KLH) is well known as the carrier. Method for conjugating KLH and peptide are also well known in the arts.

Alternatively, a gene encoding a peptide of the invention or fragment thereof may be inserted into a known expression vector, which is then used to transform a host cell as described herein. The desired peptide or fragment thereof may be recovered from the outside or inside of host cells by any standard method, and may subsequently be used as an antigen. Alternatively, whole cells expressing the peptide or their lysates or a chemically synthesized peptide may be used as the antigen.

Any mammalian animal may be immunized with the antigen, but preferably the compatibility with parental cells used for cell fusion is taken into account. In general, animals of Rodentia, Lagomorpha or Primates may be used. Animals of the family Rodentia include, for example, mouse, rat and hamster. Animals of the family Lagomorpha include, for example, rabbit. Animals of the Primate family include, for example, a monkey of Catarrhini (old world monkey) such as Macaca fascicularis, rhesus monkey, sacred baboon and chimpanzees.

Methods for immunizing animals with antigens are known in the art. Intraperitoneal injection or subcutaneous injection of antigens is a standard method for the immunization of mammals. More specifically, antigens may be diluted and suspended in an appropriate amount of phosphate buffered saline (PBS), physiological saline, etc. If desired, the antigen suspension may be mixed with an appropriate amount of a standard adjuvant, such as Freund's complete adjuvant, made into emulsion and then administered to mammalian animals. Preferably, it is followed by several administrations of antigen mixed with an appropriately amount of Freund's incomplete adjuvant every 4 to 21 days. An appropriate carrier may also be used for immunization. After immunization as above, serum may be examined by a standard method for an increase in the amount of desired antibodies.

Polyclonal antibodies against the peptides of the present invention may be prepared by collecting blood from the immunized mammal examined for the increase of desired antibodies in the serum, and by separating serum from the blood by any conventional method. Polyclonal antibodies may include serum containing the polyclonal antibodies, as well as the fraction containing the polyclonal antibodies may be isolated from the serum. Immunoglobulin G or M can be prepared from a fraction which recognizes only the peptide of the present invention using, for example, an affinity column coupled with the peptide of the present invention, and further purifying this fraction using protein A or protein G column.

To prepare monoclonal antibodies, immune cells are collected from the mammal immunized with the antigen and checked for the increased level of desired antibodies in the serum as described above, and are subjected to cell fusion. The immune cells used for cell fusion may preferably be obtained from spleen. Other preferred parental cells to be fused with the above immunocyte include, for example, myeloma cells of mammalians, and more preferably myeloma cells having an acquired property for the selection of fused cells by drugs.

The above immunocyte and myeloma cells can be fused according to known methods, for example, the method of Milstein et al. (Galfre and Milstein, Methods Enzymol 73: 3-46 (1981)).

Resulting hybridomas obtained by the cell fusion may be selected by cultivating them in a standard selection medium, such as HAT medium (hypoxanthine, aminopterin and thymidine containing medium). The cell culture is typically continued in the HAT medium for several days to several weeks, the time being sufficient to allow all the other cells, with the exception of the desired hybridoma (non-fused cells), to die. Then, the standard limiting dilution may be performed to screen and clone a hybridoma cell producing the desired antibody.

In addition to the above method, in which a non-human animal is immunized with an antigen for preparing hybridoma, human lymphocytes such as those infected by EB virus may be immunized with a peptide, peptide expressing cells or their lysates in vitro. Then, the immunized lymphocytes are fused with human-derived myeloma cells that are capable of indefinitely dividing, such as U266, to yield a hybridoma producing a desired human antibody that is able to bind to the peptide can be obtained (Unexamined Published Japanese Patent Application No. Sho 63-17688).

The obtained hybridomas are subsequently transplanted into the abdominal cavity of a mouse and the ascites are extracted. The obtained monoclonal antibodies can be purified by, for example, ammonium sulfate precipitation, a protein A or protein G column, DEAE ion exchange chromatography or an affinity column to which the peptide of the present invention is coupled. The antibody of the present invention can be used not only for purification and detection of the peptide of the present invention, but also as a candidate for agonists and antagonists of the peptide of the present invention.

Alternatively, an immune cell, such as an immunized lymphocyte, producing antibodies may be immortalized by an oncogene and used for preparing monoclonal antibodies.

Monoclonal antibodies thus obtained can be also recombinantly prepared using genetic engineering techniques (see, for example, Borrebaeck and Larrick, Therapeutic Monoclonal Antibodies, published in the United Kingdom by MacMillan Publishers LTD (1990)). For example, a DNA encoding an antibody may be cloned from an immune cell, such as a hybridoma or an immunized lymphocyte producing the antibody, inserted into an appropriate vector, and introduced into host cells to prepare a recombinant antibody. The present invention also provides recombinant antibodies prepared as described above.

Furthermore, an antibody of the present invention may be a fragment of an antibody or modified antibody, so long as it binds to one or more of the peptides of the invention. For instance, the antibody fragment may be Fab, F(ab′)₂, Fv or single chain Fv (scFv), in which Fv fragments from H and L chains are ligated by an appropriate linker (Huston et al., Proc Natl Acad Sci USA 85: 5879-83 (1988)). More specifically, an antibody fragment may be generated by treating an antibody with an enzyme, such as papain or pepsin. Alternatively, a gene encoding the antibody fragment may be constructed, inserted into an expression vector and expressed in an appropriate host cell (see, for example, Co et al., J Immunol 152: 2968-76 (1994); Better and Horwitz, Methods Enzymol 178: 476-96 (1989); Pluckthun and Skerra, Methods Enzymol 178: 497-515 (1989); Lamoyi, Methods Enzymol 121: 652-63 (1986); Rousseaux et al., Methods Enzymol 121: 663-9 (1986); Bird and Walker, Trends Biotechnol 9: 132-7 (1991)).

An antibody may be modified by conjugation with a variety of molecules, such as polyethylene glycol (PEG). The present invention provides for such modified antibodies. The modified antibody can be obtained by chemically modifying an antibody. These modification methods are conventional in the field.

Alternatively, an antibody of the present invention may be obtained as a chimeric antibody, between a variable region derived from nonhuman antibody and the constant region derived from human antibody, or as a humanized antibody, including the complementarity determining region (CDR) derived from nonhuman antibody, the frame work region (FR) and the constant region derived from human antibody. Such antibodies can be prepared according to known technology. Humanization can be performed by substituting rodent CDRs or CDR sequences for the corresponding sequences of a human antibody (see, e.g., Verhoeyen et al., Science 239:1534-1536 (1988)). Accordingly, such humanized antibodies are chimeric antibodies, wherein substantially less than an intact human variable domain has been substituted by the corresponding sequence from a non-human species.

Fully human antibodies including human variable regions in addition to human framework and constant regions can also be used. Such antibodies can be produced using various techniques known in the art. For example, in vitro methods involve use of recombinant libraries of human antibody fragments displayed on bacteriophage (e.g., Hoogenboom & Winter, J. Mol. Biol. 227:381 (1991). Similarly, human antibodies can be made by introducing of human immunoglobulin loci into transgenic animals, e.g., mice in which the endogenous immunoglobulin genes have been partially or completely inactivated. This approach is described, e.g., in U.S. Pat. Nos. 6,150,584, 5,545,807; 5,545,806; 5,569,825; 5,625,126; 5,633,425; 5,661,016.

Antibodies obtained as above may be purified to homogeneity. For example, the separation and purification of the antibody can be performed according to the separation and purification methods used for general proteins. For example, the antibody may be separated and isolated by the appropriately selected and combined use of column chromatographies, such as affinity chromatography, filter, ultrafiltration, salting-out, dialysis, SDS polyacrylamide gel electrophoresis and isoelectric focusing (Antibodies: A Laboratory Manual. Ed Harlow and David Lane, Cold Spring Harbor Laboratory (1988)), but are not limited thereto. A protein A column and protein G column can be used as the affinity column. Exemplary protein A columns to be used include, for example, Hyper D, POROS and Sepharose F.F. (Pharmacia).

Exemplary chromatography, with the exception of affinity includes, for example, ion-exchange chromatography, hydrophobic chromatography, gel filtration, reverse phase chromatography, adsorption chromatography and the like (Strategies for Protein Purification and Characterization: A Laboratory Course Manual. Ed Daniel R. Marshak et al., Cold Spring Harbor Laboratory Press (1996)). The chromatographic procedures can be carried out by liquid-phase chromatography, such as HPLC and FPLC.

For example, measurement of absorbance, enzyme-linked immunosorbent assay (ELISA), enzyme immunoassay (EIA), radioimmunoassay (RIA) and/or immunofluorescence may be used to measure the antigen binding activity of the antibody of the invention. In ELISA, the antibody of the present invention is immobilized on a plate, a peptide of the invention is applied to the plate, and then a sample containing a desired antibody, such as culture supernatant of antibody producing cells or purified antibodies, is applied. Then, a secondary antibody that recognizes the primary antibody and is labeled with an enzyme, such as alkaline phosphatase, is applied, and the plate is incubated. Next, after washing, an enzyme substrate, such as p-nitrophenyl phosphate, is added to the plate, and the absorbance is measured to evaluate the antigen binding activity of the sample. A fragment of the peptide, such as a C-terminal or N-terminal fragment, may be used as the antigen to evaluate the binding activity of the antibody. BIAcore (Pharmacia) may be used to evaluate the activity of the antibody according to the present invention.

The above methods allow for the detection or measurement of a peptide of the invention, by exposing an antibody of the invention to a sample presumed to contain a peptide of the invention, and detecting or measuring the immune complex formed by the antibody and the peptide.

Because the method of detection or measurement of the peptide according to the invention can specifically detect or measure a peptide, the method can find use in a variety of experiments in which the peptide is used.

### XV. Vectors and Host Cells

The present invention also provides a vector and host cell into which a nucleotide encoding the peptide of the present invention is introduced. A vector of the present invention may be used to keep a nucleotide, especially a DNA, of the present invention in host cell, to express a peptide of the present invention, or to administer a nucleotide of the present invention for gene therapy.

When E. coli is a host cell and the vector is amplified and produced in a large amount in E. coli (e.g., JM109, DH5 alpha, HB101 or XL1Blue), the vector should have “ori” to be amplified in E. coli and a marker gene for selecting transformed E. coli (e.g., a drug-resistance gene selected by a drug such as ampicillin, tetracycline, kanamycin, chloramphenicol or the like). For example, M13-series vectors, pUC-series vectors, pBR322, pBluescript, pCR-Script, etc., can be used. In addition, pGEM-T, pDIRECT and pT7 can also be used for subcloning and extracting cDNA as well as the vectors described above. When a vector is used to produce the protein of the present invention, an expression vector can find use. For example, an expression vector to be expressed in E. coli should have the above characteristics to be amplified in E. coli. When E. coli, such as JM109, DH5 alpha, HB101 or XL1 Blue, are used as a host cell, the vector should have a promoter, for example, lacZ promoter (Ward et al., Nature 341: 544-6 (1989); FASEB J 6: 2422-7 (1992)), araB promoter (Better et al., Science 240: 1041-3 (1988)), T7 promoter or the like, that can efficiently express the desired gene in E. coli. In that respect, pGEX-5X-1 (Pharmacia), “QIAexpress system” (Qiagen), pEGFP and pET (in this case, the host is preferably BL21 which expresses T7 RNA polymerase), for example, can be used instead of the above vectors. Additionally, the vector may also contain a signal sequence for peptide secretion. An exemplary signal sequence that directs the peptide to be secreted to the periplasm of the E. coli is the pelB signal sequence (Lei et al., J Bacteriol 169: 4379 (1987)). Means for introducing of the vectors into the target host cells include, for example, the calcium chloride method, and the electroporation method.

In addition to E. coli, for example, expression vectors derived from mammals (for example, pcDNA3 (Invitrogen) and pEGF-BOS (Nucleic Acids Res 18(17): 5322 (1990)), pEF, pCDM8), expression vectors derived from insect cells (for example, “Bac-to-BAC baculovirus expression system” (GIBCO BRL), pBacPAK8), expression vectors derived from plants (e.g., pMH1, pMH2), expression vectors derived from animal viruses (e.g., pHSV, pMV, pAdexLcw), expression vectors derived from retroviruses (e.g., pZlpneo), expression vector derived from yeast (e.g., “Pichia Expression Kit” (Invitrogen), pNV11, SP-Q01) and expression vectors derived from Bacillus subtilis (e.g., pPL608, pKTH50) can be used for producing the polypeptide of the present invention.

In order to express the vector in animal cells, such as CHO, COS or NIH3T3 cells, the vector should have a promoter necessary for expression in such cells, for example, the SV40 promoter (Mulligan et al., Nature 277: 108 (1979)), the MMLV-LTR promoter, the EF1 alpha promoter (Mizushima et al., Nucleic Acids Res 18: 5322 (1990)), the CMV promoter and the like, and preferably a marker gene for selecting transformants (for example, a drug resistance gene selected by a drug (e.g., neomycin, G418)). Examples of known vectors with these characteristics include, for example, pMAM, pDR2, pBK-RSV, pBK-CMV, pOPRSV and pOP13.

While the invention has been described in detail herein and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

### EXAMPLES

### Materials and Methods

### Experimental 1

### Cell Lines and Clinical Samples

The 23 human lung-cancer cell lines included nineteen NSCLCs (A427, A549, NCI-H1373, LC319, PC-14, PC-3, PC-9, NCI-H1666, NCI-H1781, NCI-H647, NCI-H226, NCI-H1703, NCI-H520, LU61, RERF-LC-AI, SK-MES-1, EBC-1, LX1, and NCI-H2170) and four SCLCs (DMS114, DMS273, SBC-3, and SBC-5). The human esophageal carcinoma cell lines included nine squamous cell carcinomas (SCCs: TE1, TE2, TE3, TE4, TE5, TE6, TE8, TE9, and TE10) and one adenocarcinoma (ADC: TE7). All cells were grown in monolayers in appropriate media supplemented with 10% fetal calf serum (FCS) and were maintained at 37 degrees C. in an atmosphere of humidified air with 5% CO₂.

Human small airway epithelial cells, SAEC (Cambrex Bio Science Inc., East Rutherford, N.J.) was also included in the panel of the cells used. Primary NSCLC samples had been obtained earlier with informed consent.

A24 lymphoblastoid cell line (A24LCL) was established by transformation with Epstein-bar virus into HLA-A24 positive human B lymphocyte. COS7, African green monkey kidney cell line, was purchased from ATCC.

Semiquantitative RT-PCR.

Appropriate dilutions of each single-stranded cDNA prepared from mRNAs of clinical lung and esophageal-cancer samples were prepared, taking the level of beta-actin (ACTB) expression as a quantitative control. The primer sets for amplification were as follows: ACTB-F (5′-GAGGTGATAGCATTGCTTTCG-3′) (SEQ ID NO: 152) and ACTB-R (5′-CAAGTCAGTGTACAGGTAAGC-3′) (SEQ ID NO: 153) for ACTB, C6orf167-F (5′-GTCTCACCTTGGACAGATGG-3′) (SEQ ID NO: 154) and C6orf167-R (5′-CCAAGGATCCTATTACACAGTTGC-3′) (SEQ ID NO: 155) for C6orf167.

All reactions involved initial denaturation at 95 degrees C. for 5 min followed by 22 (for ACTB) or 30 (for C6orf167) cycles of 95 degrees C. for 30 s, 56 degrees C. for 30 s, and 72 degrees C. for 60 s on a GeneAmp PCR system 9700 (Applied Biosystems, Foster City, Calif.).

Northern-Blot Analysis.

Human multiple-tissue blots (16 normal tissues including heart, brain, placenta, lung, liver, skeletal muscle, kidney, pancreas, spleen, thymus, prostate, testis, ovary, small intestine, colon, leukocyte; BD Biosciences Clontech, Palo Alto, Calif.) were hybridized with a ³²P-labeled PCR product of C6orf167. The partial-length cDNA of C6orf167 was prepared by RT-PCR using primers C6orf167-F1 (CTGGAAGAGGCAGTTGAAAA) (SEQ ID NO: 156) and C6orf167-R1 (ATCGCCCAATATACTGCTCA) (SEQ ID NO: 157). Prehybridization, hybridization, and washing were performed according to the supplier's recommendations. The blots were autoradiographed with intensifying screens at −80 degrees C. for 7 days.

Candidate Selection of Peptides Derived from C6orf167

9-mer and 10-mer peptides derived from C6orf167 that bind to HLA-A*2402 molecule were predicted using binding prediction software “BIMAS” (www-bimas.cit.nih.gov/molbio/hla_bind) (Parker et al. (J Immunol 1994, 152(1): 163-75), Kuzushima et al. (Blood 2001, 98(6): 1872-81)). These peptides were synthesized by SIGMA (Sapporo, Japan) according to a standard solid phase synthesis method and purified by reversed phase high performance liquid chromatography (HPLC). The purity (>90%) and the identity of the peptides were determined by analytical HPLC and mass spectrometry analysis, respectively. Peptides were dissolved in dimethylsulfoxide (DMSO) at 20 mg/ml and stored at −80 degrees C.

In Vitro CTL Induction

Monocyte-derived dendritic cells (DCs) were used as antigen-presenting cells (APCs) to induce cytotoxic T lymphocyte (CTL) responses against peptides presented on human leukocyte antigen (HLA). DCs were generated in vitro as described elsewhere (Nakahara S et al., Cancer Res 2003 Jul. 15, 63(14): 4112-8). Specifically, peripheral blood mononuclear cells (PBMCs) isolated from a normal volunteer (HLA-A*2402 positive) by Ficoll-Plaque (Pharmacia) solution were separated by adherence to a plastic tissue culture dish (Becton Dickinson) so as to enrich them as the monocyte fraction. The monocyte-enriched population was cultured in the presence of 1,000 U/ml of granulocyte-macrophage colony-stimulating factor (GM-CSF) (R&D System) and 1,000 U/ml of interleukin (IL)-4 (R&D System) in AIM-V Medium (Invitrogen) containing 2% heat-inactivated autologous serum (AS). After 7 days of culture, the cytokine-induced DCs were pulsed with 20 micro-g/ml of each of the synthesized peptides in the presence of 3 micro-g/ml of beta 2-microglobulin for 3 hrs at 37 degrees C. in AIM-V Medium. The generated cells appeared to express DC-associated molecules, such as CD80, CD83, CD86 and HLA class II, on their cell surfaces (data not shown). These peptide-pulsed DCs were then inactivated by X-irradiation (20 Gy) and mixed at a 1:20 ratio with autologous CD8+ T cells, obtained by positive selection with CD8 positive Isolation Kit (Dynal). These cultures were set up in 48-well plates (Corning); each well contained 1.5×10⁴ peptide-pulsed DCs, 3×10⁵ CD8+ T cells and 10 ng/ml of IL-7 (R&D System) in 0.5 ml of AIM-V/2% AS medium. Three days later, these cultures were supplemented with IL-2 (CHIRON) to a final concentration of 20 IU/ml. On days 7 and 14, the T cells were further stimulated with the autologous peptide-pulsed DCs. The DCs were prepared each time by the same way described above. CTL was tested against peptide-pulsed A24LCL cells after the 3rd round of peptide stimulation on day 21 (Tanaka H et al., Br J Cancer 2001 Jan. 5, 84(1): 94-9; Umano Y et al., Br J Cancer 2001 Apr. 20, 84(8): 1052-7; Uchida N et al., Clin Cancer Res 2004 Dec. 15, 10(24): 8577-86; Suda T et al., Cancer Sci 2006 May, 97(5): 411-9; Watanabe T et al., Cancer Sci 2005 August, 96(8): 498-506).

CTL Expansion Procedure

CTLs were expanded in culture using the method similar to the one described by Riddell et al. (Walter E A et al., N Engl J Med 1995 Oct. 19, 333(16): 1038-44; Riddell S R et al., Nat Med 1996 February, 2(2): 216-23). A total of 5×10⁴ CTLs were suspended in 25 ml of AIM-V/5% AS medium with 2 kinds of human B-lymphoblastoid cell lines, inactivated by Mitomycin C, in the presence of 40 ng/ml of anti-CD3 monoclonal antibody (Pharmingen). One day after initiating the cultures, 120 IU/ml of IL-2 were added to the cultures. The cultures were fed with fresh AIM-V/5% AS medium containing 30 IU/ml of IL-2 on days 5, 8 and 11 (Tanaka H et al., Br J Cancer 2001 Jan. 5, 84(1): 94-9; Umano Y et al., Br J Cancer 2001 Apr. 20, 84(8): 1052-7; Uchida N et al., Clin Cancer Res 2004 Dec. 15, 10(24): 8577-86; Suda T et al., Cancer Sci 2006 May, 97(5): 411-9; Watanabe T et al., Cancer Sci 2005 August, 96(8): 498-506).

Establishment of CTL Clones

The dilutions were made to have 0.3, 1, and 3 CTLs/well in 96 round-bottomed micro titer plate (Nalge Nunc International). CTLs were cultured with 1×10⁴ cells/well of 2 kinds of human B-lymphoblastoid cell lines, 30 ng/ml of anti-CD3 antibody, and 125 U/ml of IL-2 in a total of 150 micro-Dwell of AIM-V Medium containing 5% AS. 50 micro-1/well of IL-2 were added to the medium 10 days later so to reach a final concentration of 125 U/ml IL-2. CTL activity was tested on the 14th day, and CTL clones were expanded using the same method as described above (Uchida N et al., Clin Cancer Res 2004 Dec. 15, 10(24): 8577-86; Suda T et al., Cancer Sci 2006 May, 97(5): 411-9; Watanabe T et al., Cancer Sci 2005 August, 96(8): 498-506).

Specific CTL Activity

To examine specific CTL activity, interferon (IFN)-gamma enzyme-linked immunospot (ELISPOT) assay and IFN-gamma enzyme-linked immunosorbent assay (ELISA) were performed. Specifically, peptide-pulsed A24LCL (1×10⁴/well) was prepared as stimulator cells. Cultured cells in 48 wells were used as responder cells. IFN-gamma ELISPOT assay and IFN-gamma ELISA assay were performed under manufacture procedure.

Plasmid Transfection

The cDNA encoding an open reading frame of target genes or HLA-A*2402 was amplified by PCR. The PCR-amplified product was cloned into pCAGGS vector. The plasmids were transfected into COS7, which is the target genes and HLA-A24-negative cell line, using lipofectamine 2000 (Invitrogen) according to the manufacturer's recommended procedures. After 2 days from transfection, the transfected cells were harvested with versene (Invitrogen) and used as the target cells (5×10⁴ cells/well) for CTL activity assay.

Results

Enhanced C6orf167 Expression in Cancers

The wide gene expression profile data obtained from various cancers using cDNA-microarray revealed that C6orf167 (GenBank Accession No. NM_{—}198468.2; for example, SEQ ID No: 158) expression was elevated. C6orf167 expression was validly elevated in 13 out of bladder cancers, 2 out of 2 cervical cancers, 8 out of 11 cholangiocellular carcinomas, 20 out of 33 CMLs, 11 out of 15 esophageal cancers, 5 out of 8 gastric cancers, 2 out of 2 gastric diffuse-type cancers, 1 out of 2 lung cancers, 2 out of 2 lymphomas, 2 out of 3 osteosarcomas, 5 out of 12 renal carcinomas, 4 out of 4 SCLCs, 1 out of 1 soft tissue tumor and 1 out of 2 testicular tumors, as compared with corresponding normal tissue (Table 1).

TABLE 1
Ratio of cases observed up-regulation of C6orf167 in cancerous
tissue as compared with normal corresponding tissue.

| Cancer/Tumor | Ratio |
|---|---|
| Bladder Cancer | 13/17 |
| Cervical Cancer | 2/2 |
| Cholangiocellular Carcinoma | 8/11 |
| CML | 20/33 |
| Esophageal Cancer | 11/15 |
| Gastric Cancer | 5/8 |
| Gastric Diffuse-type Cancer | 2/2 |
| Lung Cancer | 1/2 |
| Lymphoma | 2/2 |
| Osteosarcoma | 2/3 |
| Renal Carcinoma | 5/12 |
| SCLC | 4/4 |
| Soft Tissue Tumor | 1/1 |
| Testicular Tumor | 1/2 |

Prediction of HLA-A24 Binding Peptides Derived from C6orf167

Tables 2a and 2b show the HLA-A24 binding 9mer and 10mer peptides of C6orf167 in the order of high binding affinity. A total of 61 peptides with potential HLA-A24 binding ability were selected and examined to determine the epitope peptides.

TABLE 2a
HLA-A24 binding 9mer peptides derived from
C6orf167

| | | Start | Amino | | SEQ |
|---|---|---|---|---|---|
| | | Posi- | Acid | Binding | ID |
| Peptide name | Rank | tion | sequence | Score | NO. |
| C6orf167-A24-9mer | 1 | 848 | EYMKQLVKL | 330 | 1 |
| | 2 | 179 | LYIGHLSEL | 330 | 2 |
| | 3 | 641 | LYPSHEKLL | 300 | 3 |
| | 4 | 404 | MYLHCCLTL | 300 | 4 |
| | 5 | 1171 | YYYQVYSIL | 280 | 5 |
| | 6 | 1219 | AYSKLLSHL | 240 | 6 |
| | 7 | 236 | VYGHQFMNL | 240 | 7 |
| | 8 | 480 | SYTIFLCIL | 200 | 8 |
| | 9 | 1170 | RYYYQVYSI | 100 | 9 |
| | 10 | 580 | MYAQKNLDI | 50 | 10 |
| | 11 | 203 | LFPPSWHLL | 36 | 11 |
| | 12 | 254 | LFEEHCETL | 36 | 12 |
| | 13 | 158 | PYEALEAQL | 36 | 13 |
| | 14 | 9 | TFLTDSLEL | 33 | 14 |
| | 15 | 561 | AFVTSQRAL | 30 | 15 |
| | 16 | 530 | NFFSLFLLL | 28.8 | 16 |
| | 17 | 966 | LFRIIDCLL | 28 | 17 |
| | 18 | 315 | SFWNWLNKL | 26.4 | 18 |
| | 19 | 92 | LFHLFRQQL | 24 | 19 |
| | 20 | 95 | LFRQQLYNL | 20 | 20 |
| | 21 | 786 | RYLSHVLQN | 15 | 21 |
| | 22 | 132 | LFLHYVKVF | 15 | 22 |
| | 23 | 598 | AFREKAKEF | 13.2 | 23 |
| | 24 | 827 | KNLSGPDDL | 12 | 24 |
| | 25 | 851 | KQLVKLTRL | 12 | 25 |
| | 26 | 55 | RLILNLDPL | 12 | 26 |
| | 27 | 626 | IYIDGVQEV | 11.88 | 27 |
| | 28 | 908 | KSAMVTKSL | 11.2 | 28 |
| | 29 | 550 | HVLDLLNFL | 10.368 | 29 |
| | 30 | 220 | LVLEILYML | 10.08 | 30 |
| | 31 | 437 | SFSISWLPF | 10 | 31 |

Start position indicates the number of amino acid residue from the N-terminus of C6orf167.
Binding score is derived from “BIMAS”.

TABLE 2b
HLA-A24 binding lOmer peptides derived from
C6orf167

| | | Start | Amino | | SEQ |
|---|---|---|---|---|---|
| | | Posi- | Acid | Binding | ID |
| Peptide name | Rank | tion | sequence | Score | NO. |
| C6orf167-A24-10mer | 1 | 1170 | RYYYqVYSIL | 560 | 32 |
| | 2 | 626 | IYIDgVQEVF | 252 | 33 |
| | 3 | 429 | YYSKnLNSSF | 120 | 34 |
| | 4 | 917 | EYLGeVLKYI | 105 | 35 |
| | 5 | 474 | LYKSsSSYTI | 50 | 36 |
| | 6 | 514 | KFHQkRMEEL | 44 | 37 |
| | 7 | 254 | LFEEhCETLL | 36 | 38 |
| | 8 | 194 | AFVNqNQIKL | 33 | 39 |
| | 9 | 240 | QFMNlASDNL | 30 | 40 |
| | 10 | 956 | IFATsKAQKL | 26.4 | 41 |
| | 11 | 786 | RYLShVLQNS | 25.2 | 42 |
| | 12 | 511 | IYSKfHQKRM | 25 | 43 |
| | 13 | 315 | SFWNwLNKLL | 24 | 44 |
| | 14 | 598 | AFREkAKEFL | 24 | 45 |
| | 15 | 869 | IFSKaQVEYL | 20 | 46 |
| | 16 | 966 | LFRIiDCLLL | 20 | 47 |
| | 17 | 66 | NFEEdTLEIF | 18 | 48 |
| | 18 | 914 | KSLEyLGEVL | 17.28 | 49 |
| | 19 | 964 | KLLFrIIDCL | 16.8 | 50 |
| | 20 | 143 | RYLKvQNAES | 16.5 | 51 |
| | 21 | 647 | KLLNdGFSML | 14.4 | 52 |
| | 22 | 851 | KQLVkLTRLL | 14.4 | 53 |
| | 23 | 519 | RMEE1TEVGL | 14.4 | 54 |
| | 24 | 97 | RQQLyNLETL | 12 | 55 |
| | 25 | 827 | KNLSgPDDLL | 12 | 56 |
| | 26 | 389 | KSISvQGVIL | 12 | 57 |
| | 27 | 273 | RYDKvRSSES | 11 | 58 |
| | 28 | 670 | SFLQaVLARI | 10.5 | 59 |
| | 29 | 132 | LFLHyVKVFI | 10.5 | 60 |
| | 30 | 1112 | LLLPgILKCL | 10.08 | 61 |

Start position indicates the number of amino acid residue from the N-terminus of C6orf167.
Binding score is derived from “BIMAS”.

CTL Induction with the Predicted Peptides from C6orf167 Restricted with HLA-A*2402 and Establishment for CTL Lines Stimulated with C6orf167 Derived Peptides

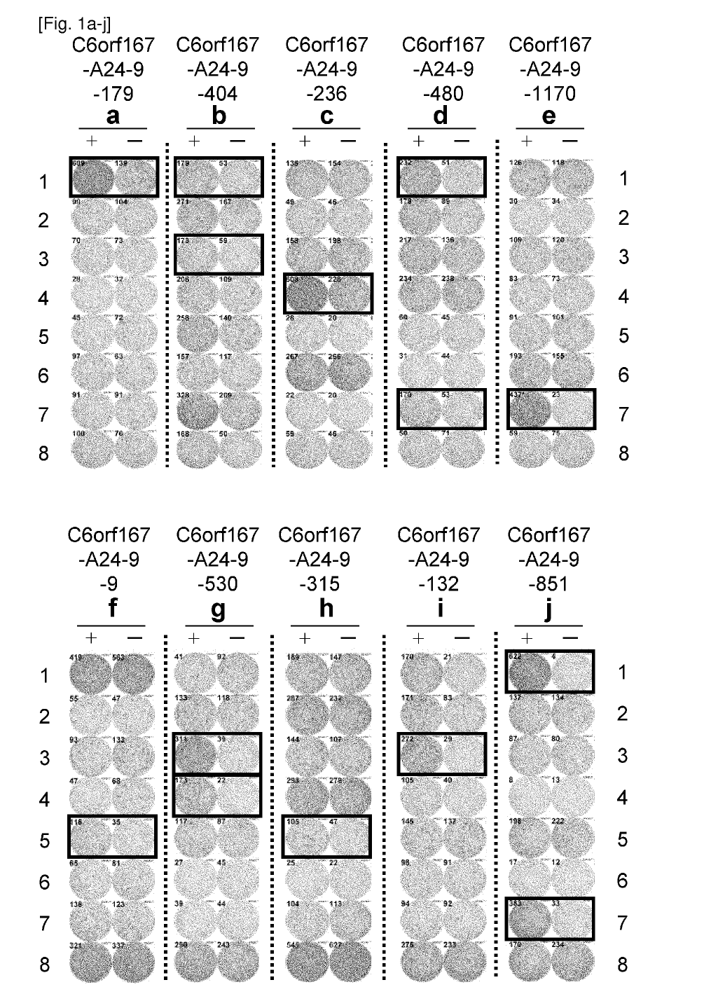

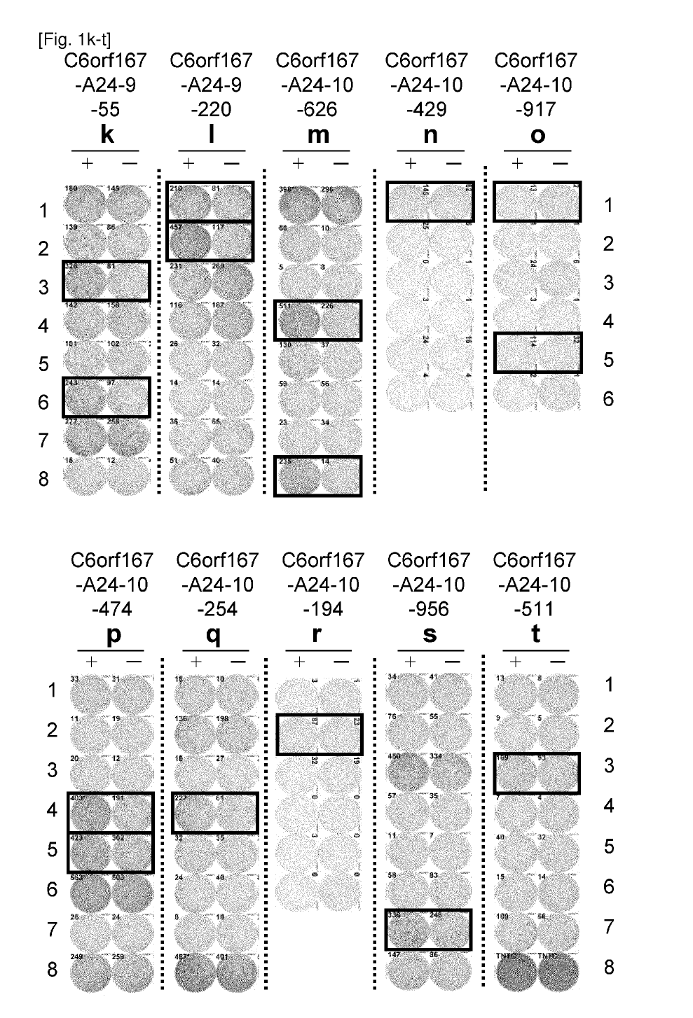

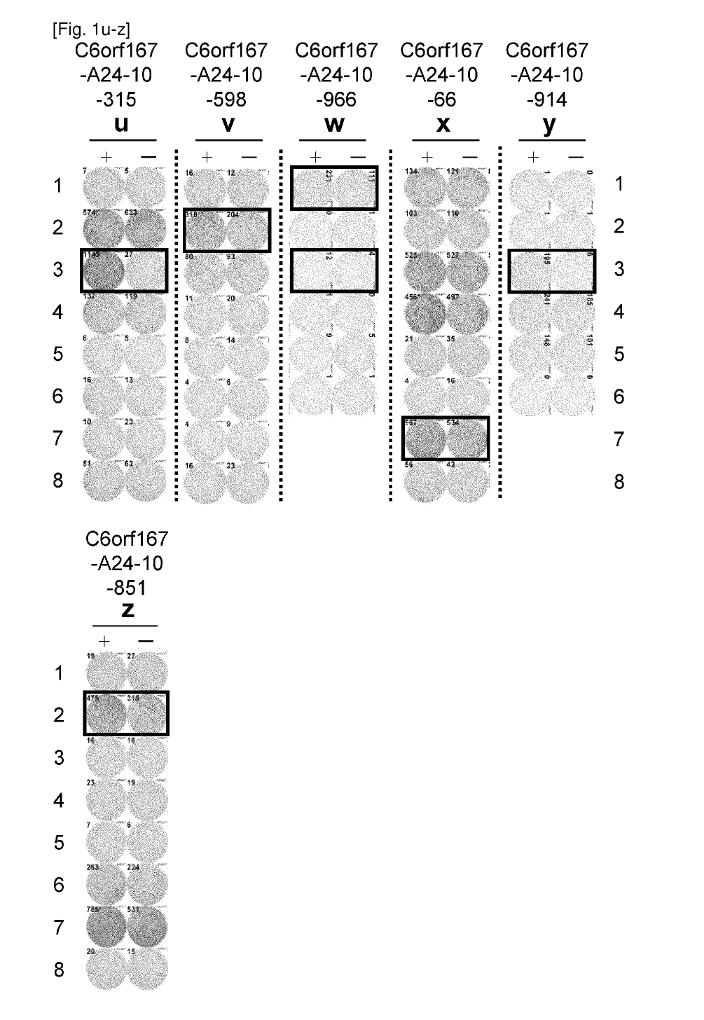

CTLs for those peptides derived from C6orf167 were generated according to the protocols as described in “Materials and Methods”. Peptide specific CTL activity was determined by IFN-gamma ELISPOT assay (FIGS. 1a-z). The following well numbers demonstrated potent IFN-gamma production as compared to the control wells:

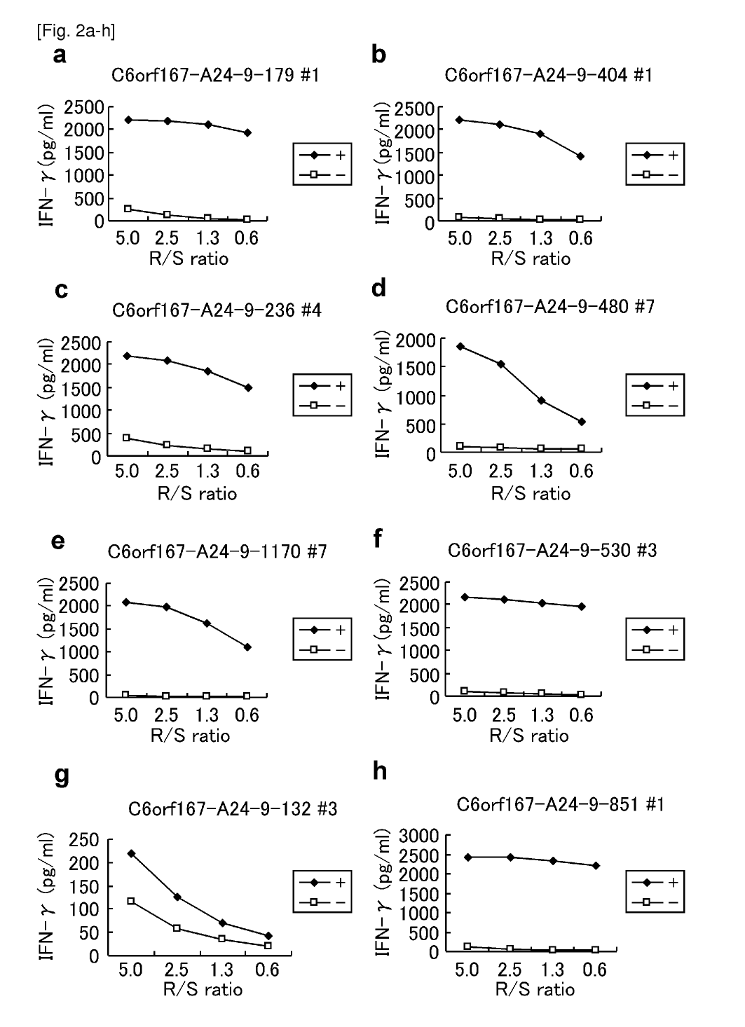

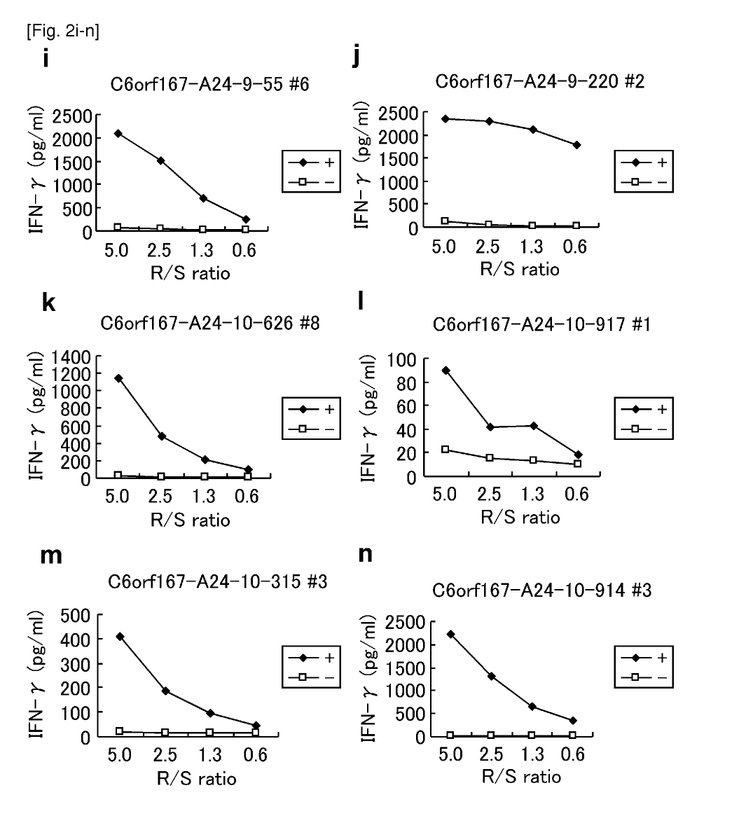

#1 stimulated with C6orf167-A24-9-179 (SEQ ID NO: 2) (a), #1 and #3 with C6orf167-A24-9-404 (SEQ ID NO: 4) (b), #4 with C6orf167-A24-9-236 (SEQ ID NO: 7) (c), #1 and #7 with C6orf167-A24-9-480 (SEQ ID NO: 8) (d), #7 with C6orf167-A24-9-1170 (SEQ ID NO: 9) (e), #5 with C6orf167-A24-9-9 (SEQ ID NO: 14) (f), #3 and #4 with C6orf167-A24-9-530 (SEQ ID NO: 16) (g), #5 with C6orf167-A24-9-315 (SEQ ID NO: 18) (h), #3 with C6orf167-A24-9-132 (SEQ ID NO: 22) (i), #1 and #7 with C6orf167-A24-9-851 (SEQ ID NO: 25) (j), #3 and #6 with C6orf167-A24-9-55 (SEQ ID NO: 26) (k), #1 and #2 with C6orf167-A24-9-220 (SEQ ID NO: 30) (l), #4 and #8 with C6orf167-A24-10-626 (SEQ ID NO: 33) (m), #1 with C6orf167-A24-10-429 (SEQ ID NO: 34) (n), #1 and #5 with C6orf167-A24-10-917 (SEQ ID NO: 35) (o), #4 and #5 with C6orf167-A24-10-474 (SEQ ID NO: 36) (p), #4 with C6orf167-A24-10-254 (SEQ ID NO: 38) (q), #2 with C6orf167-A24-10-194 (SEQ ID NO: 39) (r), #7 with C6orf167-A24-10-956 (SEQ ID NO: 41) (s), #3 with C6orf167-A24-10-511 (SEQ ID NO: 43) (t), #3 with C6orf167-A24-10-315 (SEQ ID NO: 44) (u), #2 with C6orf167-A24-10-598 (SEQ ID NO: 45) (v), #1 and #3 with C6orf167-A24-10-966 (SEQ ID NO: 47) (w), #7 with C6orf167-A24-10-66 (SEQ ID NO: 48) (x), #3 with C6orf167-A24-10-914 (SEQ ID NO: 49) (y) and #2 with C6orf167-A24-10-851 (SEQ ID NO: 53) (z). Furthermore, the cells in the positive well number #1 stimulated with C6orf167-A24-9-179 (SEQ ID NO: 2), #1 with C6orf167-A24-9-404 (SEQ ID NO: 4), #4 with C6orf167-A24-9-236 (SEQ ID NO: 7), #7 with C6orf167-A24-9-480 (SEQ ID NO: 8), #7 with C6orf167-A24-9-1170 (SEQ ID NO: 9), #3 with C6orf167-A24-9-530 (SEQ ID NO: 16), #3 with C6orf167-A24-9-132 (SEQ ID NO: 22), #1 with C6orf167-A24-9-851 (SEQ ID NO: 25), #6 with C6orf167-A24-9-55 (SEQ ID NO: 26), #2 with C6orf167-A24-9-220 (SEQ ID NO: 30), #8 with C6orf167-A24-10-626 (SEQ ID NO: 33), #1 with C6orf167-A24-10-917 (SEQ ID NO: 35), #3 with C6orf167-A24-10-315 (SEQ ID NO: 44) and #3 with C6orf167-A24-10-914 (SEQ ID NO: 49) were expanded and established CTL lines. CTL activity of those CTL lines was determined by IFN-gamma ELISA assay (FIGS. 2a-n). It showed that all CTL lines demonstrated potent IFN-gamma production against the target cells pulsed with corresponding peptide as compared to target cells without peptide pulse. On the other hand, no potent IFN-gamma production could be detected by stimulation with other peptides shown in Table 2, despite those peptide had possible binding activity with HLA-A*2402 (data not shown). As a result, it indicated that 26 peptides derived from C6orf167 were screened as the peptides could induce potent CTLs.

Establishment of CTL Clones Against C6orf167 Specific Peptides

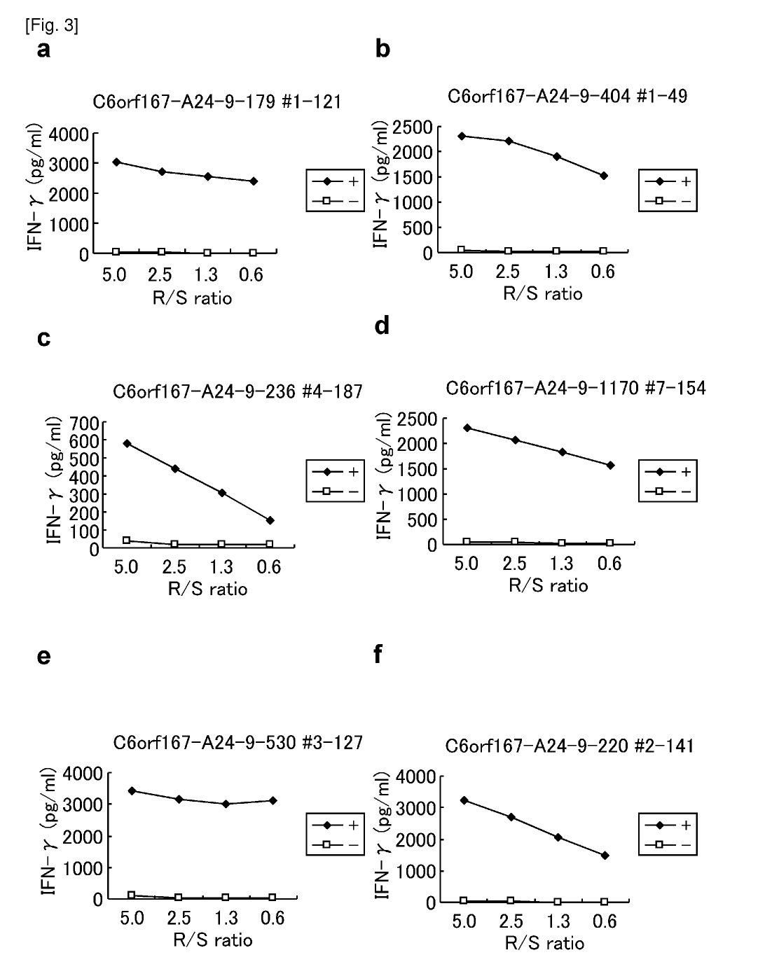

CTL clones were established by limiting dilution from CTL lines as described in “Materials and Methods”, and IFN-gamma production from CTL clones against target cells pulsed peptide were determined by IFN-gamma ELISA assay. Potent IFN-gamma productions were determined from CTL clones stimulated with C6orf167-A24-9-179 (SEQ ID NO: 2) (a), C6orf167-A24-9-404 (SEQ ID NO: 4) (b), C6orf167-A24-9-236 (SEQ ID NO: 7) (c), C6orf167-A24-9-1170 (SEQ ID NO: 9) (d), C6orf167-A24-9-530 (SEQ ID NO: 16) (e) and C6orf167-A24-9-220 (SEQ ID NO: 30) (f) in FIG. 3.

Specific CTL Activity Against Target Cells Exogenously Expressing C6orf167 and HLA-A*2402

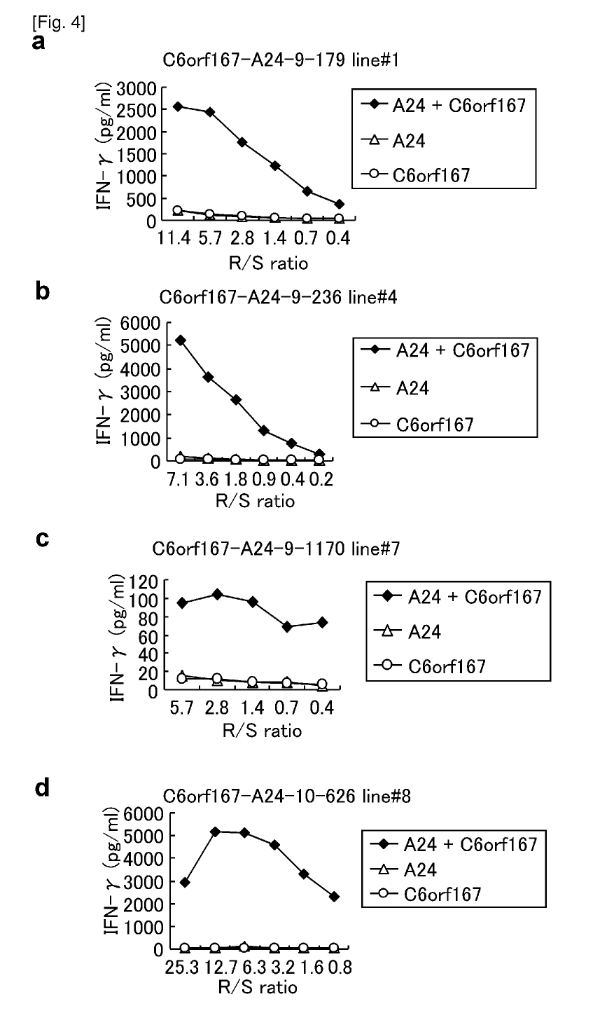

The established CTL lines raised against these peptides were examined for their ability to recognize target cells that endogenously express C6orf167 and HLA-A*2402 molecule. Specific CTL activity against COS7 cells which transfected with both the full length of C6orf167 and HLA-A*2402 molecule gene (a specific model for the target cells that exogenously express C6orf167 and HLA-A*2402 gene) was tested using the CTL lines raised by corresponding peptide as the effecter cells. COS7 cells transfected with either full length of C6orf167 genes or HLA-A*2402 were prepared as controls. In FIG. 4, the CTLs stimulated with C6orf167-A24-9-179 (SEQ ID NO: 2), C6orf167-A24-9-236 (SEQ ID NO: 7), C6orf167-A24-9-1170 (SEQ ID NO: 9) and C6orf167-A24-10-626 (SEQ ID NO: 33) showed potent CTL activity against COS7 cells expressing both C6orf167 and HLA-A*2402. On the other hand, no significant specific CTL activity was detected against the controls. Thus, these data clearly demonstrated that peptides of C6orf167-A24-9-179 (SEQ ID NO: 2), C6orf167-A24-9-236 (SEQ ID NO: 7), C6orf167-A24-9-1170 (SEQ ID NO: 9) and C6orf167-A24-10-626 (SEQ ID NO: 33) were endogenously processed and expressed on the target cells with HLA-A*2402 molecule and were recognized by the CTLs. The result indicate that these peptides derived from C6orf167 may be suitable as cancer vaccines for patients with C6orf167 expressing tumors.

Homology Analysis of Antigen Peptides

The CTLs stimulated with C6orf167-A24-9-179 (SEQ ID NO: 2), C6orf167-A24-9-404 (SEQ ID NO: 4), C6orf167-A24-9-236 (SEQ ID NO: 7), C6orf167-A24-9-480 (SEQ ID NO: 8), C6orf167-A24-9-1170 (SEQ ID NO: 9), C6orf167-A24-9-9 (SEQ ID NO: 14), C6orf167-A24-9-530 (SEQ ID NO: 16), C6orf167-A24-9-315 (SEQ ID NO: 18), C6orf167-A24-9-132 (SEQ ID NO: 22), C6orf167-A24-9-851 (SEQ ID NO: 25), C6orf167-A24-9-55 (SEQ ID NO: 26), C6orf167-A24-9-220 (SEQ ID NO: 30), C6orf167-A24-10-626 (SEQ ID NO: 33), C6orf167-A24-10-429 (SEQ ID NO: 34), C6orf167-A24-10-917 (SEQ ID NO: 35), C6orf167-A24-10-474 (SEQ ID NO: 36), C6orf167-A24-10-254 (SEQ ID NO: 38), C6orf167-A24-10-194 (SEQ ID NO: 39), C6orf167-A24-10-956 (SEQ ID NO: 41), C6orf167-A24-10-511 (SEQ ID NO: 43), C6orf167-A24-10-315 (SEQ ID NO: 44), C6orf167-A24-10-598 (SEQ ID NO: 45), C6orf167-A24-10-966 (SEQ ID NO: 47), C6orf167-A24-10-66 (SEQ ID NO: 48), C6orf167-A24-10-914 (SEQ ID NO: 49) and C6orf167-A24-10-851 (SEQ ID NO: 53) showed significant and specific CTL activity. This result may be due to the fact that the sequences of C6orf167-A24-9-179 (SEQ ID NO: 2), C6orf167-A24-9-404 (SEQ ID NO: 4), C6orf167-A24-9-236 (SEQ ID NO: 7), C6orf167-A24-9-480 (SEQ ID NO: 8), C6orf167-A24-9-1170 (SEQ ID NO: 9), C6orf167-A24-9-9 (SEQ ID NO: 14), C6orf167-A24-9-530 (SEQ ID NO: 16), C6orf167-A24-9-315 (SEQ ID NO: 18), C6orf167-A24-9-132 (SEQ ID NO: 22), C6orf167-A24-9-851 (SEQ ID NO: 25), C6orf167-A24-9-55 (SEQ ID NO: 26), C6orf167-A24-9-220 (SEQ ID NO: 30), C6orf167-A24-10-626 (SEQ ID NO: 33), C6orf167-A24-10-429 (SEQ ID NO: 34), C6orf167-A24-10-917 (SEQ ID NO: 35), C6orf167-A24-10-474 (SEQ ID NO: 36), C6orf167-A24-10-254 (SEQ ID NO: 38), C6orf167-A24-10-194 (SEQ ID NO: 39), C6orf167-A24-10-956 (SEQ ID NO: 41), C6orf167-A24-10-511 (SEQ ID NO: 43), C6orf167-A24-10-315 (SEQ ID NO: 44), C6orf167-A24-10-598 (SEQ ID NO: 45), C6orf167-A24-10-966 (SEQ ID NO: 47), C6orf167-A24-10-66 (SEQ ID NO: 48), C6orf167-A24-10-914 (SEQ ID NO: 49) and C6orf167-A24-10-851 (SEQ ID NO: 53) are homologous to peptides derived from other molecules that are known to sensitize the human immune system. To exclude this possibility, homology analyses were performed for these peptide sequences using as queries the BLAST algorithm (www.ncbi.nlm.nih.gov/blast/blast.cgi) which revealed no sequence with significant homology. The results of homology analyses indicate that the sequences of C6orf167-A24-9-179 (SEQ ID NO: 2), C6orf167-A24-9-404 (SEQ ID NO: 4), C6orf167-A24-9-236 (SEQ ID NO: 7), C6orf167-A24-9-480 (SEQ ID NO: 8), C6orf167-A24-9-1170 (SEQ ID NO: 9), C6orf167-A24-9-9 (SEQ ID NO: 14), C6orf167-A24-9-530 (SEQ ID NO: 16), C6orf167-A24-9-315 (SEQ ID NO: 18), C6orf167-A24-9-132 (SEQ ID NO: 22), C6orf167-A24-9-851 (SEQ ID NO: 25), C6orf167-A24-9-55 (SEQ ID NO: 26), C6orf167-A24-9-220 (SEQ ID NO: 30), C6orf167-A24-10-626 (SEQ ID NO: 33), C6orf167-A24-10-429 (SEQ ID NO: 34), C6orf167-A24-10-917 (SEQ ID NO: 35), C6orf167-A24-10-474 (SEQ ID NO: 36), C6orf167-A24-10-254 (SEQ ID NO: 38), C6orf167-A24-10-194 (SEQ ID NO: 39), C6orf167-A24-10-956 (SEQ ID NO: 41), C6orf167-A24-10-511 (SEQ ID NO: 43), C6orf167-A24-10-315 (SEQ ID NO: 44), C6orf167-A24-10-598 (SEQ ID NO: 45), C6orf167-A24-10-966 (SEQ ID NO: 47), C6orf167-A24-10-66 (SEQ ID NO: 48), C6orf167-A24-10-914 (SEQ ID NO: 49) and C6orf167-A24-10-851 (SEQ ID NO: 53) are unique and thus, there is little possibility, to our best knowledge, that these molecules raise unintended immunologic response to some unrelated molecule.

In conclusion, novel HLA-A24 epitope peptides derived from C6orf167 were identified. Furthermore, it was demonstrated that epitope peptides of C6orf167 may be applicable for cancer immunotherapy.

Expression of C6orf167 in Lung Cancers, Esophageal Cancers and Normal Tissues.

Using a cDNA microarray to screen for elements that were highly transactivated in a large proportion of lung cancer (WO2007/013665) and/or esophageal cancers, the C6orf167 gene was identified as a good candidate target for diagnosing and/or treating cancers. This gene showed a higher level of expression in the majority of lung and esophageal cancers. Subsequently it was confirmed its transactivation by semiquantitative RT-PCR experiments in 7 of 10 NSCLC cases (3 of 5 ADCs and 4 of 5 SCCs) and in all of 5 SCLC cases (FIG. 5A, right upper panels) as well as in all of 10 NSCLC cell lines and all of 5 SCLC cell lines (FIG. 5A, right lower panels). Up-regulation of C6orf167 was also detected in 7 of 10 esophageal cancer cases and 9 of 10 esophageal cancer cell lines (FIG. 5A, left upper and lower panels).

Northern-blot analysis using C6orf167 cDNA as a probe identified 7.5-kb transcript, exclusively in the testis and small intestine among 16 human tissues examined (FIG. 5B).

### Experimental 2

### Cell Lines

T2, HLA-A*0201-positive B-lymphoblastoid cell line, and COS7, African green monkey kidney cell line, were purchased from ATCC.

Candidate Selection of Peptides Derived from C6orf167

9-mer and 10-mer peptides derived from C6orf167 that bind to HLA-A*0201 molecule were predicted using binding prediction software “BIMAS” (www-bimas.cit.nih.gov/molbio/hla_bind) (Parker et al. (J Immunol 1994, 152(1): 163-75), Kuzushima et al. (Blood 2001, 98(6): 1872-81)). These peptides were synthesized by Biosynthesis (Lewisville, Tex.) according to a standard solid phase synthesis method and purified by reversed phase high performance liquid chromatography (HPLC). The purity (>90%) and the identity of the peptides were determined by analytical HPLC and mass spectrometry analysis, respectively. Peptides were dissolved in dimethylsulfoxide at 20 mg/ml and stored at −80 degrees C.

In Vitro CTL Induction

Monocyte-derived dendritic cells (DCs) were used as antigen-presenting cells to induce cytotoxic T lymphocyte (CTL) responses against peptides presented on human leukocyte antigen (HLA). DCs were generated in vitro as described elsewhere (Nakahara S et al., Cancer Res 2003 Jul. 15, 63(14): 4112-8). Specifically, peripheral blood mononuclear cells (PBMCs) isolated from a normal volunteer (HLA-A*0201 positive) by Ficoll-Plaque (Pharmacia) solution were separated by adherence to a plastic tissue culture dish (Becton Dickinson) so as to enrich them as the monocyte fraction. The monocyte-enriched population was cultured in the presence of 1,000 U/ml of granulocyte-macrophage colony-stimulating factor (R&D System) and 1,000 U/ml of interleukin (IL)-4 (R&D System) in AIM-V Medium (Invitrogen) containing 2% heat-inactivated autologous serum (AS). After 7 days of culture, the cytokine-induced DCs were pulsed with 20 micro-g/ml of each of the synthesized peptides in the presence of 3 micro-g/ml of beta 2-microglobulin for 3 hrs at 37 degrees C. in AIM-V Medium. The generated cells appeared to express DC-associated molecules, such as CD80, CD83, CD86 and HLA class II, on their cell surfaces (data not shown). These peptide-pulsed DCs were then inactivated by X-irradiated (20 Gy) and mixed at a 1:20 ratio with autologous CD8+ T cells, obtained by positive selection with CD8 positive Isolation Kit (Dynal). These cultures were set up in 48-well plates (Corning); each well contained 1.5×10⁴ peptide-pulsed DCs, 3×10⁵ CD8+ T cells and 10 ng/ml of IL-7 (R&D System) in 0.5 ml of AIM-V/2% AS medium. Three days later, these cultures were supplemented with IL-2 (CHIRON) to a final concentration of 20 IU/ml. On days 7 and 14, the T cells were further stimulated with the autologous peptide-pulsed DCs. The DCs were prepared each time by the same way described above. CTL was tested against peptide-pulsed T2 cells after the 3rd round of peptide stimulation on day 21 (Tanaka H et al., Br J Cancer 2001 Jan. 5, 84(1): 94-9; Umano Y et al., Br J Cancer 2001 Apr. 20, 84(8): 1052-7; Uchida N et al., Clin Cancer Res 2004 Dec. 15, 10(24): 8577-86; Suda T et al., Cancer Sci 2006 May, 97(5): 411-9; Watanabe T et al., Cancer Sci 2005 August, 96(8): 498-506).

CTL Expansion Procedure

CTLs were expanded in culture using the method similar to the one described by Riddell et al. (Walter E A et al., N Engl J Med 1995 Oct. 19, 333(16): 1038-44; Riddell S R et al., Nat Med 1996 February, 2(2): 216-23). A total of 5×10⁴ CTLs were suspended in 25 ml of AIM-V/5% AS medium with 2 kinds of human B-lymphoblastoid cell lines, inactivated by Mitomycin C, in the presence of 40 ng/ml of anti-CD3 monoclonal antibody (Pharmingen). One day after initiating the cultures, 120 IU/ml of IL-2 were added to the cultures. The cultures were fed with fresh AIM-V/5% AS medium containing 30 IU/ml of IL-2 on days 5, 8 and 11 (Tanaka H et al., Br J Cancer 2001 Jan. 5, 84(1): 94-9; Umano Y et al., Br J Cancer 2001 Apr. 20, 84(8): 1052-7; Uchida N et al., Clin Cancer Res 2004 Dec. 15, 10(24): 8577-86; Suda T et al., Cancer Sci 2006 May, 97(5): 411-9; Watanabe T et al., Cancer Sci 2005 August, 96(8): 498-506).

Establishment of CTL Clones

The dilutions were made to have 0.3, 1, and 3 CTLs/well in 96 round-bottomed micro titer plate (Nalge Nunc International). CTLs were cultured with 1×10⁴ cells/well of 2 kinds of human B-lymphoblastoid cell lines, 30 ng/ml of anti-CD3 antibody, and 125 U/ml of IL-2 in a total of 150 micro-l/well of AIM-V Medium containing 5% AS. 50 micro-1/well of IL-2 were added to the medium 10 days later so to reach a final concentration of 125 U/ml IL-2. CTL activity was tested on the 14th day, and CTL clones were expanded using the same method as described above (Uchida N et al., Clin Cancer Res 2004 Dec. 15, 10(24): 8577-86; Suda T et al., Cancer Sci 2006 May, 97(5): 411-9; Watanabe T et al., Cancer Sci 2005 August, 96(8): 498-506).

Specific CTL Activity

To examine specific CTL activity, interferon (IFN)-gamma enzyme-linked immunospot (ELISPOT) assay and IFN-gamma enzyme-linked immunosorbent assay (ELISA) were performed. Specifically, peptide-pulsed T2 (1×10⁴/well) was prepared as stimulator cells. Cultured cells in 48 wells were used as responder cells. IFN-gamma ELISPOT assay and IFN-gamma ELISA assay were performed under manufacture procedure.

Establishment of the Cells Forcibly Expressing Either or Both of the Target Gene and HLA-A02

The cDNA encoding an open reading frame of target genes or HLA-A*0201 was amplified by PCR. The PCR-amplified product was cloned into pCAGGS vector and pIRES vector (Clontech Laboratories, Inc., Cat. No. 631605). The plasmids were transfected into COS7, which is the target genes and HLA-A*0201-null cell line, using lipofectamine 2000 (Invitrogen) according to the manufacturer's recommended procedures. After 2 days from transfection, the transfected cells were harvested with versene (Invitrogen) and used as the target cells (5×10⁴ cells/well) for CTL activity assay.

Results

Prediction of HLA-A02 Binding Peptides Derived from C6orf167

Tables 3a and 3b show the HLA-A02 binding 9mer and 10mer peptides of C6orf167 in the order of high binding affinity. A total of 90 peptides with potential HLA-A02 binding ability were selected and examined to determine the epitope peptides.

TABLE 3a
HLA-A2 binding 9mer peptides derived from
C6orf167

| | Amino | | SEQ |
|---|---|---|---|
| Start | acid | Binding | ID |
| Position | sequence | Score | NO |
| 918 | YLGEVLKYI | 2489.0 | 62 |
| 202 | KLFPPSWHL | 2074.8 | 63 |
| 227 | MLGEKLKQV | 1115.0 | 64 |
| 855 | KLTRLLFNL | 997.4 | 65 |
| 131 | VLFLHYVKV | 656.2 | 66 |
| 533 | SLFLLLAAV | 591.9 | 67 |
| 858 | RLLFNLSEV | 591.9 | 68 |
| 290 | CLCIKELWV | 382.5 | 69 |
| 647 | KLLNDGFSM | 373.1 | 70 |
| 929 | YLGKKVFSA | 304.9 | 71 |
| 219 | WLVLEILYM | 289.1 | 72 |
| 904 | TLSDKSAMV | 285.2 | 73 |
| 648 | LLNDGFSML | 282.9 | 74 |
| 133 | FLHYVKVFI | 264.0 | 75 |
| 887 | ALVRFFEAV | 246.1 | 76 |
| 319 | WLNKLLKTL | 226.0 | 77 |
| 667 | TVLSFLQAV | 196.8 | 78 |
| 261 | TLLCDLISL | 181.8 | 79 |
| 965 | LLFRIIDCL | 151.4 | 80 |
| 964 | KLLFRIIDC | 148.0 | 81 |
| 578 | LLMYAQKNL | 134.4 | 82 |
| 623 | LLSIYIDGV | 133.3 | 83 |
| 484 | FLCILAKVV | 131.2 | 84 |
| 457 | SMLEMVKTC | 125.2 | 85 |
| 253 | SLFEEHCET | 113.0 | 86 |
| 671 | FLQAVLARI | 110.4 | 87 |
| 283 | LMSDQCPCL | 107.5 | 88 |
| 1018 | YLNQLLGNV | 95.7 | 89 |
| 1091 | SILAFILQL | 95.0 | 90 |
| 1113 | LLPGILKCL | 83.5 | 91 |
| 821 | VLQMYIKNL | 83.5 | 92 |
| 1116 | GILKCLVLV | 81.4 | 93 |
| 528 | LQNFFSLFL | 77.0 | 94 |
| 1112 | LLLPGILKC | 71.9 | 95 |
| 99 | QLYNLETLL | 68.4 | 96 |
| 590 | VLAEKFSCA | 65.8 | 97 |
| 224 | ILYMLGEKL | 56.9 | 98 |
| 405 | YLHCCLTLC | 52.6 | 99 |

TABLE 3b
HLA-A2 binding 10mer peptides derived from
C6orf167

| | Amino | | SEQ |
|---|---|---|---|
| Start | acid | Binding | ID |
| Position | sequence | Score | NO |
| 716 | ALWRhFFSFL | 7040.9 | 100 |
| 535 | FLLLaAVAEV | 2722.7 | 101 |
| 226 | YMLGeKLKQV | 1966.4 | 102 |
| 303 | LLDHrSKWFV | 1950.9 | 103 |
| 311 | FVSEsFWNWL | 1252.6 | 104 |
| 425 | ILWEyYSKNL | 1235.8 | 105 |
| 554 | LLNFlKPAFV | 650.3 | 106 |
| 648 | LLNDgFSMLL | 565.8 | 107 |
| 569 | LIWKgHMAFL | 524.3 | 108 |
| 202 | KLFPpSWHLL | 470.3 | 109 |
| 527 | GLQNfFSLFL | 446.5 | 110 |
| 10 | FLTDsLELEL | 402.9 | 111 |
| 577 | FLLMyAQKNL | 363.6 | 112 |
| 128 | QQCVlFLHYV | 339.0 | 113 |
| 622 | TLLSiYIDGV | 290.0 | 114 |
| 178 | LLYIgHLSEL | 267.3 | 115 |
| 47 | YLCSgALKRL | 226.0 | 116 |
| 219 | WLVLeILYML | 226.0 | 117 |
| 1155 | QLTSvFRQFI | 218.0 | 118 |
| 227 | MLGEkLKQVV | 198.8 | 119 |
| 253 | SLFEeHCETL | 158.8 | 120 |
| 606 | FLVSkNEEMV | 156.8 | 121 |
| 290 | CLCIkELWVL | 151.1 | 122 |
| 262 | LLCDlISLSL | 148.9 | 123 |
| 965 | LLFRiIDCLL | 134.4 | 124 |
| 1113 | LLPGiLKCLV | 118.2 | 125 |
| 77 | IQWVtETALV | 99.5 | 126 |
| 319 | WLNKlLKTLL | 98.3 | 127 |
| 1022 | LLGNvIEQYI | 97.5 | 128 |
| 910 | AMVTkSLEYL | 95.6 | 129 |
| 738 | QLADaAADFT | 82.3 | 130 |
| 482 | TIFLcILAKV | 81.4 | 131 |
| 282 | SLMSdQCPCL | 79.0 | 132 |
| 442 | WLPFkGLANT | 78.8 | 133 |
| 625 | SIYIdGVQEV | 70.4 | 134 |
| 1120 | CLVLvSEPQV | 69.6 | 135 |
| 640 | CLYPsHEKLL | 68.4 | 136 |
| 619 | TIWTlLSIYI | 65.5 | 137 |
| 747 | TLLAmDMPST | 63.4 | 138 |
| 1131 | RLATeNLQYM | 62.8 | 139 |
| 71 | TLEIfGIQWV | 56.3 | 140 |
| 614 | MVQRqTIWTL | 54.8 | 141 |
| 457 | SMLEmVKTCC | 54.4 | 142 |
| 1001 | YLQGmCIVCC | 52.6 | 143 |
| 397 | ILEEqLRMYL | 52.4 | 144 |
| 268 | SLSLnRYDKV | 51.1 | 145 |
| 1088 | RLASiLAFIL | 50.8 | 146 |
| 528 | LQNFfSLFLL | 49.1 | 147 |
| 1049 | VLLAlRNTAT | 46.9 | 148 |
| 886 | KALVrFFEAV | 44.3 | 149 |
| 411 | TLCDfWEPNI | 42.8 | 150 |
| 579 | LMYAqKNLDI | 41.0 | 151 |

CTL Induction with the Predicted Peptides from C6orf167 Restricted with HLA-A*0201

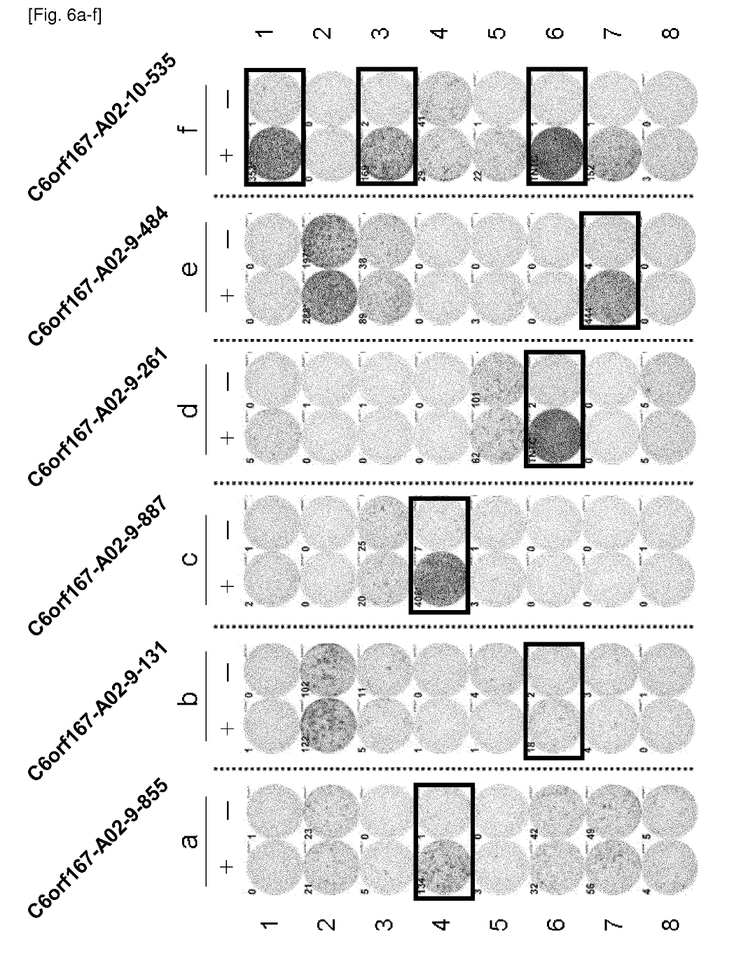

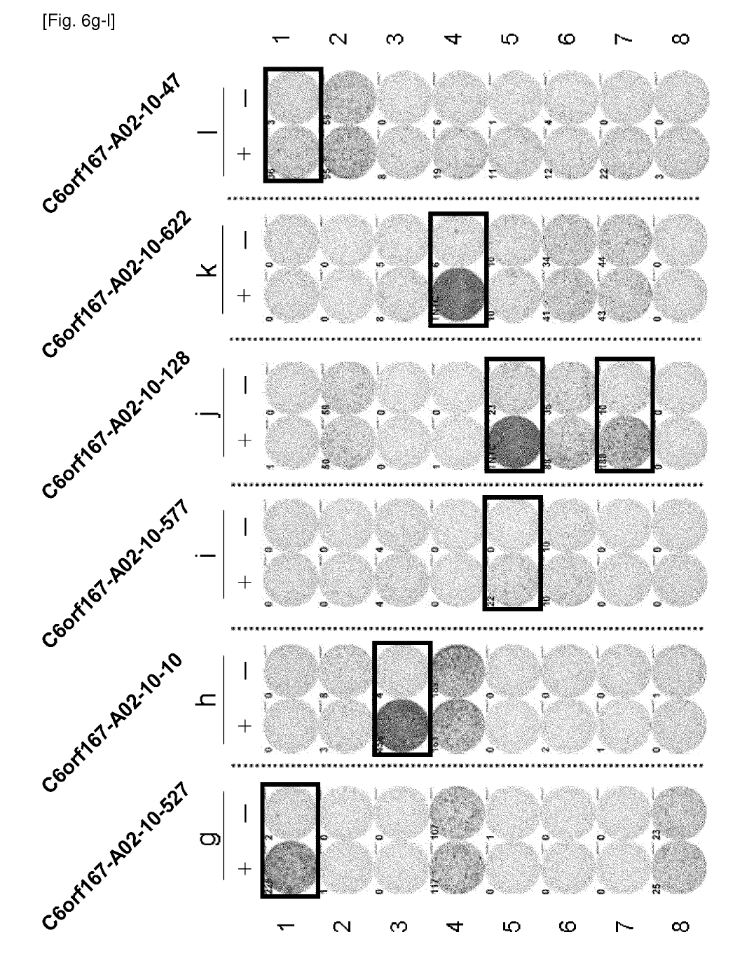

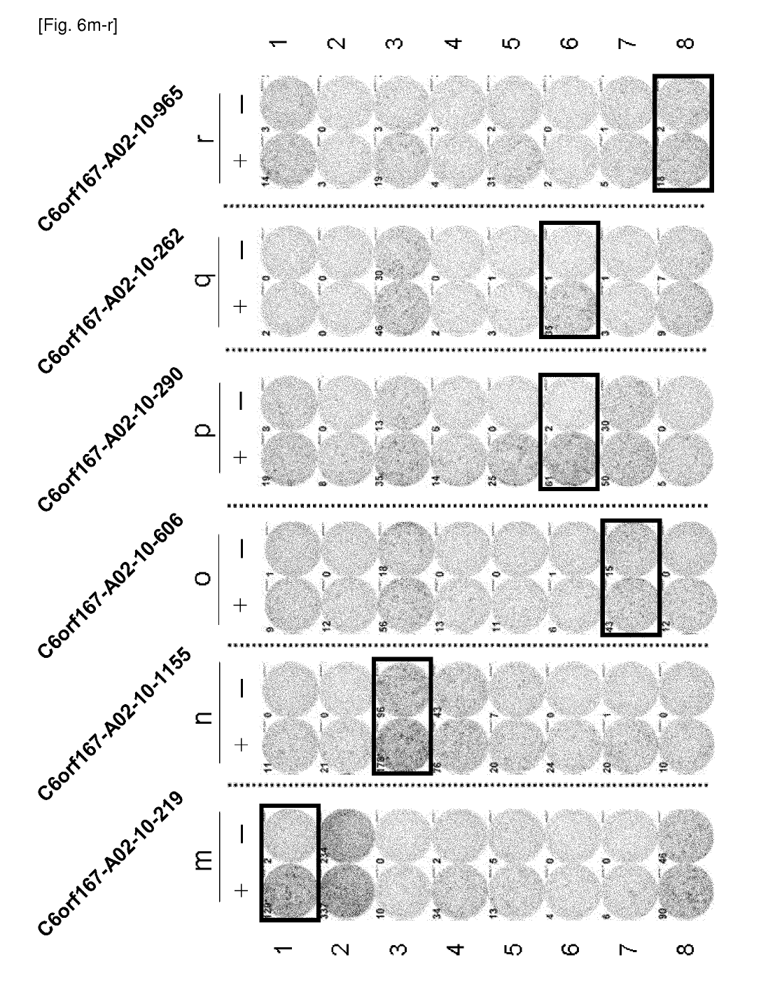

CTLs for those peptides derived from C6orf167 were generated according to the protocols as described in “Materials and Methods”. Peptide specific CTL activity was determined by IFN-gamma ELISPOT assay (FIGS. 6a-r). The following well numbers demonstrated potent IFN-gamma production as compared to the control wells: #4 with C6orf167-A02-9-855 (SEQ ID NO: 65) (a), #6 with C6orf167-A02-9-131 (SEQ ID NO: 66) (b), #4 with C6orf167-A02-9-887 (SEQ ID NO: 76) (c), #6 with C6orf167-A02-9-261 (SEQ ID NO: 79) (d), #7 with C6orf167-A02-9-484 (SEQ ID NO: 84) (e), #1, #3 and #6 with C6orf167-A02-10-535 (SEQ ID NO: 101) (f), #1 with C6orf167-A02-10-527 (SEQ ID NO: 110) (g), #3 with C6orf167-A02-10-10 (SEQ ID NO: 111) (h), #5 with C6orf167-A02-10-577 (SEQ ID NO: 112) (i), #5 and #7 with C6orf167-A02-10-128 (SEQ ID NO: 113) (j), #4 with C6orf167-A02-10-622 (SEQ ID NO: 114) (k), #1 with C6orf167-A02-10-47 (SEQ ID NO: 116) (l), #1 with C6orf167-A02-10-219 (SEQ ID NO: 117) (m), #3 with C6orf167-A02-10-1155 (SEQ ID NO: 118) (n), #7 with C6orf167-A02-10-606 (SEQ ID NO: 121) (o), #6 with C6orf167-A02-10-290 (SEQ ID NO: 122) (p), #6 with C6orf167-A02-10-262 (SEQ ID NO: 123) (q) and #8 with C6orf167-A02-10-965 (SEQ ID NO: 124) (r). On the other hand, no specific CTL activity was observed by stimulation with other peptides shown in Tables 3a and 3b, despite those peptides had possible binding activity with HLA-A*0201. As a typical case of negative data, it was not shown specific IFN-gamma production from the CTL stimulated with C6orf167-A02-9-918 (SEQ ID NO: 62) (s). As a result, it indicated that 18 peptides derived from C6orf167 was screened as the peptides that could induce potent CTLs.

Establishment of CTL Lines and Clones Against C6orf167 Derived Peptide

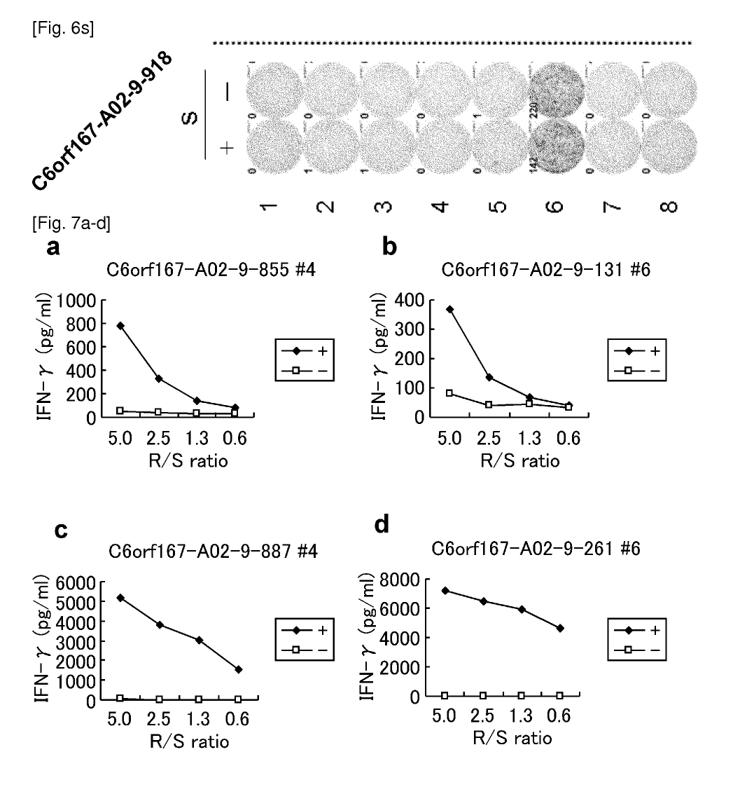

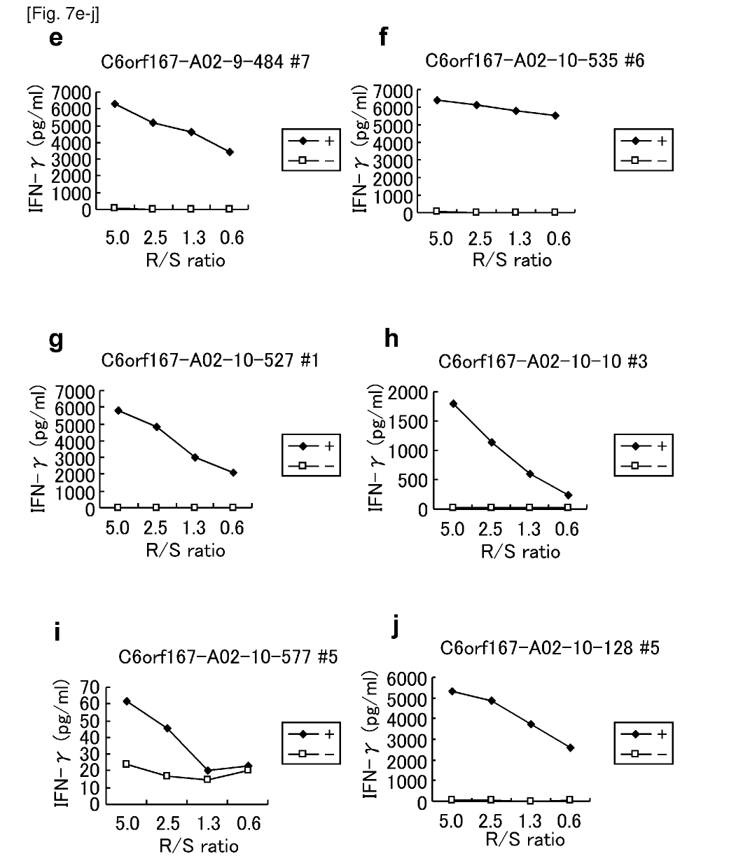

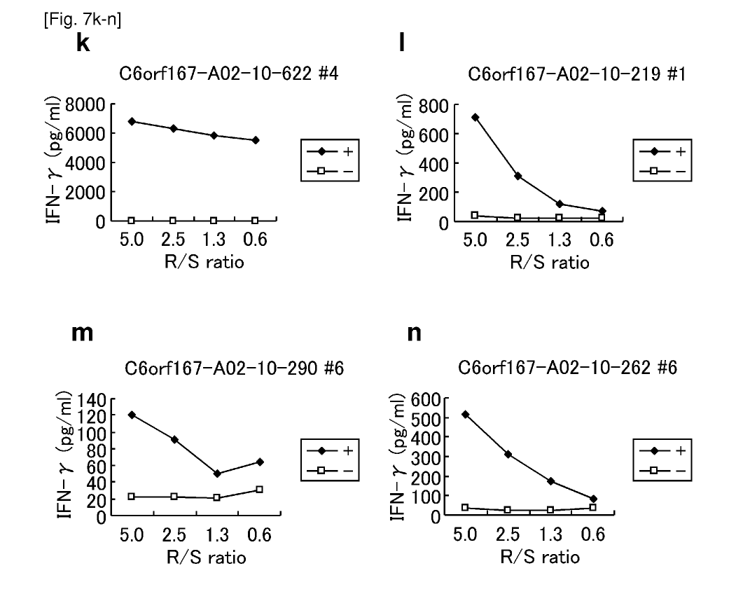

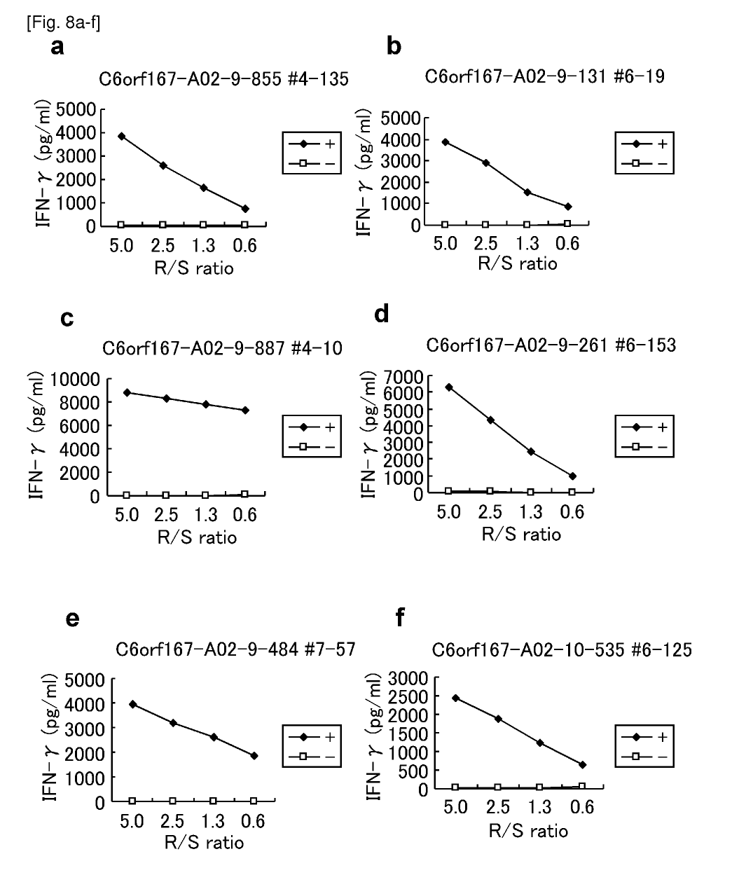

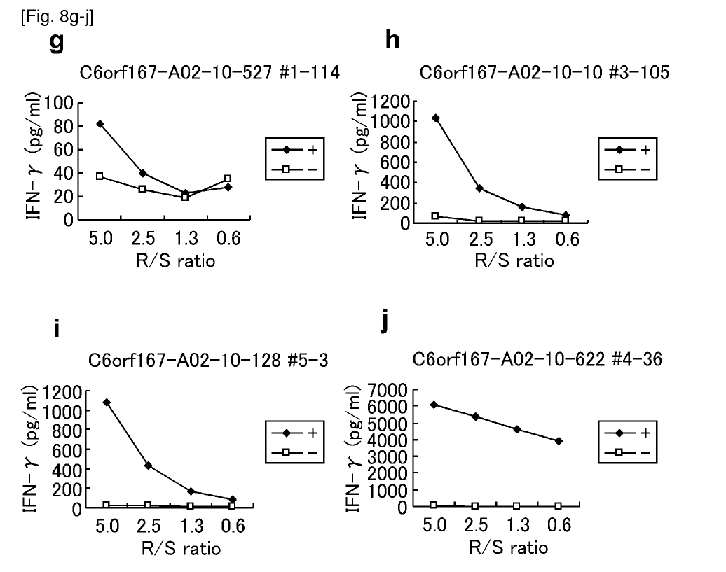

The cells that showed peptide specific CTL activity detected by IFN-gamma ELISPOT assay in the well number #4 with C6orf167-A02-9-855 (SEQ ID NO: 65) (a), #6 with C6orf167-A02-9-131 (SEQ ID NO: 66) (b), #4 with C6orf167-A02-9-887 (SEQ ID NO: 76) (c), #6 with C6orf167-A02-9-261 (SEQ ID NO: 79) (d), #7 with C6orf167-A02-9-484 (SEQ ID NO: 84) (e), #6 with C6orf167-A02-10-535 (SEQ ID NO: 101) (f), #1 with C6orf167-A02-10-527 (SEQ ID NO: 110) (g), #3 with C6orf167-A02-10-10 (SEQ ID NO: 111) (h), #5 with C6orf167-A02-10-577 (SEQ ID NO: 112) (i), #5 with C6orf167-A02-10-128 (SEQ ID NO: 113) (j), #4 with C6orf167-A02-10-622 (SEQ ID NO: 114) (k), #1 with C6orf167-A02-10-219 (SEQ ID NO: 117) (l), #6 with C6orf167-A02-10-290 (SEQ ID NO: 122) (m) and #6 with C6orf167-A02-10-262 (SEQ ID NO: 123) (n) were expanded and established the CTL lines. CTL activity of these CTL lines was determined by IFN-gamma ELISA assay (FIGS. 7a-n). The results show that the CTL lines demonstrated potent IFN-gamma production against the target cells pulsed with the corresponding peptide as compared to target cells without peptide pulse. Furthermore, the CTL clones were established by limiting dilution from the CTL lines as described in “Materials and Methods”, and IFN-gamma production from the CTL clones against target cells pulsed peptide was determined by IFN-gamma ELISA assay. Potent IFN-gamma productions were determined from the CTL clones stimulated with C6orf167-A02-9-855 (SEQ ID NO: 65) (a), C6orf167-A02-9-131 (SEQ ID NO: 66) (b), C6orf167-A02-9-887 (SEQ ID NO: 76) (c), C6orf167-A02-9-261 (SEQ ID NO: 79) (d), C6orf167-A02-9-484 (SEQ ID NO: 84) (e), C6orf167-A02-10-535 (SEQ ID NO: 101) (f), C6orf167-A02-10-527 (SEQ ID NO: 110) (g), C6orf167-A02-10-10 (SEQ ID NO: 111) (h), C6orf167-A02-10-128 (SEQ ID NO: 113) (i) and C6orf167-A02-10-622 (SEQ ID NO: 114) (j) (FIGS. 8a-j).

Specific CTL Activity Against Target Cells Exogenously Expressing C6orf167 and HLA-A*0201

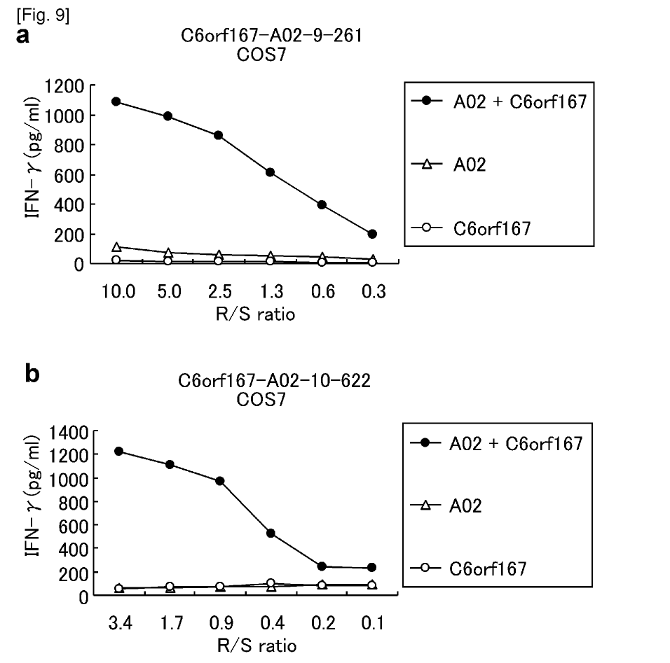

The established CTL lines and clones raised against each peptide were examined for the ability to recognize target cells that endogenously express C6orf167 and HLA-A*0201 molecule. Specific CTL activity against COS7 cells which transfected with both the full length of C6orf167 and HLA-A*0201 gene (a specific model for the target cells that exogenously express C6orf167 and HLA-A*0201 gene) was tested by using the CTL lines and clones raised by corresponding peptide as the effector cells. COS7 cells transfected with either full length of C6orf167 or HLA-A*0201 were prepared as the controls. In FIG. 9, the CTL line stimulated with C6orf167-A02-9-261 (SEQ ID NO: 79) (a) and the CTL clone stimulated with C6orf167-A02-10-622 (SEQ ID NO: 114) (b) showed potent CTL activity against COS7 cells expressing both C6orf167 and HLA-A*0201. On the other hand, no significant specific CTL activity was detected against the controls. Thus, these data clearly demonstrated that peptides of C6orf167-A02-9-261 (SEQ ID NO: 79) and C6orf167-A02-10-622 (SEQ ID NO: 114) were endogenously processed and expressed on the target cells with HLA-A*0201 molecule and were recognized by the CTLs. These results indicated that these peptides derived from C6orf167 may be available to apply the cancer vaccines for patients with C6orf167 expressing tumors.

Homology Analysis of Antigen Peptides

The CTLs stimulated with C6orf167-A02-9-855 (SEQ ID NO: 65), C6orf167-A02-9-131 (SEQ ID NO: 66), C6orf167-A02-9-887 (SEQ ID NO: 76), C6orf167-A02-9-261 (SEQ ID NO: 79), C6orf167-A02-9-484 (SEQ ID NO: 84), C6orf167-A02-10-535 (SEQ ID NO: 101), C6orf167-A02-10-527 (SEQ ID NO: 110), C6orf167-A02-10-10 (SEQ ID NO: 111), C6orf167-A02-10-577 (SEQ ID NO: 112), C6orf167-A02-10-128 (SEQ ID NO: 113), C6orf167-A02-10-622 (SEQ ID NO: 114), C6orf167-A02-10-47 (SEQ ID NO: 116), C6orf167-A02-10-219 (SEQ ID NO: 117), C6orf167-A02-10-1155 (SEQ ID NO: 118), C6orf167-A02-10-606 (SEQ ID NO: 121), C6orf167-A02-10-290 (SEQ ID NO: 122), C6orf167-A02-10-262 (SEQ ID NO: 123) and C6orf167-A02-10-965 (SEQ ID NO: 124) showed significant and specific CTL activity. This result may be due to the fact that the sequence of C6orf167-A02-9-855 (SEQ ID NO: 65), C6orf167-A02-9-131 (SEQ ID NO: 66), C6orf167-A02-9-887 (SEQ ID NO: 76), C6orf167-A02-9-261 (SEQ ID NO: 79), C6orf167-A02-9-484 (SEQ ID NO: 84), C6orf167-A02-10-535 (SEQ ID NO: 101), C6orf167-A02-10-527 (SEQ ID NO: 110), C6orf167-A02-10-10 (SEQ ID NO: 111), C6orf167-A02-10-577 (SEQ ID NO: 112), C6orf167-A02-10-128 (SEQ ID NO: 113), C6orf167-A02-10-622 (SEQ ID NO: 114), C6orf167-A02-10-47 (SEQ ID NO: 116), C6orf167-A02-10-219 (SEQ ID NO: 117), C6orf167-A02-10-1155 (SEQ ID NO: 118), C6orf167-A02-10-606 (SEQ ID NO: 121), C6orf167-A02-10-290 (SEQ ID NO: 122), C6orf167-A02-10-262 (SEQ ID NO: 123) and C6orf167-A02-10-965 (SEQ ID NO: 124) are homologous to peptide derived from other molecules that are known to sensitize the human immune system. To exclude this possibility, homology analyses were performed for this peptide sequence using as queries the BLAST algorithm (www.ncbi.nlm.nih.gov/blast/blast.cgi) which revealed no sequence with significant homology. The results of homology analyses indicate that the sequence of C6orf167-A02-9-855 (SEQ ID NO: 65), C6orf167-A02-9-131 (SEQ ID NO: 66), C6orf167-A02-9-887 (SEQ ID NO: 76), C6orf167-A02-9-261 (SEQ ID NO: 79), C6orf167-A02-9-484 (SEQ ID NO: 84), C6orf167-A02-10-535 (SEQ ID NO: 101), C6orf167-A02-10-527 (SEQ ID NO: 110), C6orf167-A02-10-10 (SEQ ID NO: 111), C6orf167-A02-10-577 (SEQ ID NO: 112), C6orf167-A02-10-128 (SEQ ID NO: 113), C6orf167-A02-10-622 (SEQ ID NO: 114), C6orf167-A02-10-47 (SEQ ID NO: 116), C6orf167-A02-10-219 (SEQ ID NO: 117), C6orf167-A02-10-1155 (SEQ ID NO: 118), C6orf167-A02-10-606 (SEQ ID NO: 121), C6orf167-A02-10-290 (SEQ ID NO: 122), C6orf167-A02-10-262 (SEQ ID NO: 123) and C6orf167-A02-10-965 (SEQ ID NO: 124) are unique and thus, there is little possibility, to our best knowledge, that this molecules raise unintended immunologic response to some unrelated molecule.

In conclusion, novel HLA-A*0201 epitope peptides derived from C6orf167 were identified. Furthermore, it was demonstrated that epitope peptides of C6orf167 may be applicable for cancer immunotherapy.

### INDUSTRIAL APPLICABILITY

The present invention provides new TAAs, particularly those derived from C6orf167 which may induce potent and specific anti-tumor immune responses and have applicability to a wide variety of cancer types. Such TAAs can find utility as peptide vaccines against diseases associated with C6orf167, e.g., cancer, examples of which include, but are not limited to, bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor.

1.-2. (canceled)

3. An isolated peptide of (a) or (b) below:
(a) an isolated peptide comprising an amino acid sequence selected from the group consisting of SEQ ID NOs: 2, 4, 7, 8, 9, 14, 16, 18, 22, 25, 26, 30, 33, 34, 35, 36, 38, 39, 41, 43, 44, 45, 47, 48, 49, 53, 65, 66, 76, 79, 84, 101, 110, 111, 112, 113, 114, 117, 118, 121, 122, 123, and 124;
(b) an isolated peptide comprising an amino acid sequence selected from the group consisting of SEQ ID NOs: 2, 4, 7, 8, 9, 14, 16, 18, 22, 25, 26, 30, 33, 34, 35, 36, 38, 39, 41, 43, 44, 45, 47, 48, 49, 53, 65, 66, 76, 79, 84, 101, 110, 111, 112, 113, 114, 117, 118, 121, 122, 123, and 124 in which 1, 2, or several amino acid(s) are substituted deleted or added, wherein the peptide has CTL inducibility and binds to an HLA antigen.

4. The isolated peptide of claim 3, wherein said peptide is a nonapeptide or a decapeptide.

5. (canceled)

6. The isolated peptide of claim 3, having one or both of the following characteristics:
(a) the second amino acid from the N-terminus is or is modified to be an amino acid selected from the group consisting of phenylalanine, tyrosine, methionine, and tryptophan, and
(b) the C-terminal amino acid is or is modified to be an amino acid selected from the group consisting of phenylalanine, leucine, isoleucine, tryptophan, and methionine.

7. The isolated peptide of claim 3, which, in the context of HLA-A2, has at least one substitution selected from the group consisting of:
(a) the second amino acid from the N-terminus is selected from the group consisting of leucine and methionine; and
(b) the C-terminal amino acid is selected from the group consisting of valine and leucine.

8. An isolated polynucleotide encoding the isolated peptide of claim 3.

9. A composition for inducing CTL, wherein the composition comprises the one or more of the peptide(s) of claim 3, or one or more of the polynucleotide(s) encoding the peptide.

10. A pharmaceutical composition for the treatment and/or prophylaxis of cancer, and/or the prevention of the postoperative recurrence thereof, wherein the composition comprises the peptide of claim 3, or a polynucleotide encoding the peptide.

11. The pharmaceutical composition of claim 10 formulated for the administration to a subject whose HLA antigen is HLA-A*2402 or A*0201.

12. A method for inducing an antigen-presenting cell (APC) with CTL inducibility comprising the step selected from the group consisting of:
(a) contacting an APC with a peptide as set forth in claim 3 in vitro, ex vivo or in vivo, and
(b) introducing a polynucleotide encoding the peptide as set forth in claim 3 into an APC.

13. A method for inducing CTL comprising a step selected from the group consisting of:
(a) co-culturing a CD8 positive T cell with an APC, which presents on its surface a complex of an HLA antigen and the peptide as set forth in claim 3,
(b) co-culturing a CD8 positive T cell with an exosome, which presents on its surface a complex of an HLA antigen and the peptide of claim 3, and
(c) introducing a gene that comprises a polynucleotide encoding a T cell receptor (TCR) subunit polypeptide binding to the peptide of claim 3 into a T cell.

14. An isolated APC that presents on its surface a complex of an HLA antigen and the peptide of claim 3.

15. An isolated APC that presents on its surface a complex of an HLA antigen and the peptide of claim 3, which is induced by a method comprising the step selected from the group consisting of:
(a) contacting an APC with a peptide as set forth in claim 3 in vitro, ex vivo or in vivo, and
(b) introducing a polynucleotide encoding the peptide as set forth in claim 3 into an APC.

16. An isolated CTL that targets the peptide of claim 3.

17. An isolated CTL that targets the peptide of claim 3, which is induced a method comprising a step selected from the group consisting of:
(a) co-culturing a CD8 positive T cell with an APC, which presents on its surface a complex of an HLA antigen and the peptide as set forth in claim 3,
(b) co-culturing a CD8 positive T cell with an exosome, which presents on its surface a complex of an HLA antigen and the peptide of claim 3, and
(c) introducing a gene that comprises a polynucleotide encoding a T cell receptor (TCR) subunit polypeptide binding to the peptide of claim 3 into a T cell.

18. A method of inducing immune response against cancer in a subject comprising administering to the subject a composition comprising the peptide as set forth in claim 3, an immunologically active fragment thereof, or a polynucleotide encoding the peptide or the fragment.

19. A method for diagnosing cancer, said method comprising the steps of:
(a) determining the expression level of a gene in a subject-derived biological sample by a method selected from the group consisting of:
(i) detecting an mRNA of C6orf167 gene,
(ii) detecting a protein encoded by C6orf167 gene, and
(iii) detecting biological activity of a protein encoded by C6orf167 gene; and
(b) correlating an increase in the expression level determined in step (a) as compared to a normal control level of the gene to presence of cancer.

20. The method of claim 19, wherein the expression level determined in step (a) is at least 10% greater than the normal control level.

21. The method of claim 19, wherein the cancer is selected from the group of bladder cancer, cervical cancer, cholangiocellular carcinoma, chronic myelogenous leukemia (CML), esophageal cancer, gastric cancer, gastric diffuse-type cancer, lung cancer, lymphoma, osteosarcoma, renal carcinoma, lung adenocarcinoma (ADC), lung squamous cell carcinoma (SCC), small-cell lung cancer (SCLC), non-small-cell lung cancer (NSCLC), soft tissue tumor and testicular tumor.

22. The method of claim 19, wherein the expression level determined in step (a) is determined by detecting hybridization of a probe to the mRNA of the C6orf167 gene.

23. The method of claim 19, wherein the expression level determined in step (a) is determined by detecting the binding of an antibody against the protein of C6orf167.

24. The method of claim 19, wherein the subject-derived biological sample comprises biopsy, sputum, blood, pleural effusion or urine.

25. A kit for use in diagnosing cancer, said kit comprising a reagent selected from the group consisting of:
(a) a reagent for detecting an mRNA of C6orf167 gene,
(b) a reagent for detecting a protein encoded by C6orf167 gene, and
(c) a reagent for detecting biological activity of a protein encoded by C6orf167 gene.

26. (canceled)

27. An antibody or fragment thereof against the peptide of claim 3.

28. A vector comprising a nucleotide sequence encoding the peptide of claim 3.

29. A diagnostic kit comprising the peptide of claim 3, a polynucleotide encoding the peptide, or an antibody or fragment thereof against the peptide.

30. The isolated peptide of claim 3, wherein the HLA antigen is HLA-A24 or HLA-A2.
